(19) United States
(12) Patent Application Publication
(10) Pub. No.: US 2008/0050709 A1
(43) Pub. Date: Feb. 28, 2008

(54) INTEGRATED BLOOD SUGAR CONTROL, BLOOD PRESSURE CONTROL AND HEART FAILURE SELF-CARE SYSTEM AND METHOD

(75) Inventors: Sylvia Aruffo, Highland Park, IL (US); Shirley Grey, Northbrook, IL (US); Carol Outland, Carol Stream, IL (US); Judith Franks Farah, Northbrook, IL (US)

Correspondence Address: BELL, BOYD & LLOYD, LLP, P.O. Box 1135, CHICAGO, IL, 60690, US

(73) Assignee: CAREGUIDE SYSTEMS, INC., Buffalo Grove, IL (US)

(21) Appl. No.: 11/771,793

(22) Filed: Jun. 29, 2007

Related U.S. Application Data
(60) Provisional application No. 60/806,201, filed on Jun. 29, 2006.
(60) Provisional application No. 60/806,200, filed on Jun. 29, 2006.
(60) Provisional application No. 60/806,203, filed on Jun. 29, 2006.

(51) Int. Cl. G09B 23/28; A47B 81/00; A61B 5/00

(52) U.S. Cl. 434/262; 312/234; 600/485

(57) ABSTRACT

Methods and apparatus for blood sugar control, blood pressure control and heart failure care are disclosed. The methods and apparatus enable a user to provide blood sugar control, blood pressure control, and heart failure care to himself/herself. For example, after being treated by a doctor for a heart problem, a person may use the disclosed system to prevent additional heart related problems.

### PRIORITY CLAIM

This application is a non-provisional of, claims priority to and the benefit of U.S. Provisional Patent Application Ser. No. 60/806,201 filed Jun. 29, 2006; U.S. Provisional Patent Application Ser. No. 60/806,200 filed Jun. 29, 2006; and U.S. Provisional Patent Application Ser. No. 60/806,203 filed Jun. 29, 2006, the entire contents of which are incorporated herein by reference.

### COPYRIGHT NOTICE

A portion of the disclosure of this patent document contains material which is subject to copyright protection. The copyright owner has no objection to the photocopy reproduction by anyone of the patent document or the patent disclosure in exactly the form it appears in the Patent and Trademark Office patent file or records, but otherwise reserves all copyright rights whatsoever.

### TECHNICAL FIELD

The present application relates in general to methods, systems and apparatus for integrated blood sugar control, blood pressure control and heart failure self-care by a person.

### BACKGROUND

The health care system in the United States has dramatically changed in the last several decades. Lengthy hospital stays after surgical and other medical procedures have decreased or have been eliminated, at least in part, to reduce overall health care cost. When patients leave hospitals or do not stay in hospitals, the patients often require more professional home care and/or more self-care at home. Self-care at home has necessitated the need for better, more advanced, more user friendly, and more easier-to-use self-care systems, apparatus and methods. For example, the assignee of the present application has developed and distributed many individual self-care systems. Such self-care systems have typically been designed for self-care after an individual surgical or other medical procedure (such as the Post Surgery Drain System described in U.S. Pat. No. 6,926,708).

Self-care systems, apparatus and methods have also been distributed for individual medical conditions (such as for asthma control, blood pressure control, blood sugar control, and healthy foot care). The assignee of the present application has developed and marketed several individual self-care systems, each focused solely on self-care for an individual medical condition.

However, many people have multiple, different, simultaneously existing medical conditions. Multiple, different, simultaneously existing medical conditions can be related to each other, or may be unrelated to each other. For instance, a person may have heart disease and high blood pressure. These conditions may be considered to be related, in a sense, because they both. relate to the heart. On the other hand, a person may have asthma and high blood pressure. These conditions may be, considered to be in a sense, unrelated.

Patients with multiple, different, simultaneously existing medical conditions can use the known individual self-care systems to individually care for each of their conditions. For instance, a person with heart disease and high blood pressure may use an individual heart disease self-care system and may also use an individual high blood pressure self-care system. While these self-care systems may have some overlapping instructions, they may also have contradictory or inconsistent instructions. The concurrent use of these two individual different self-care systems may also require the person to duplicate certain efforts. Using two or more different self-care systems may also cause the person to become confused, discouraged, or overwhelmed. Moreover, many people tend to be able to handle only a limited amount of information and instructions. Using such multiple individual systems greatly lessens the likelihood that the patient will strictly follow either of the self-care systems. Moreover, even if a person is compliant with both individual self-care systems, the systems may be contradictory to a certain extent or may not be effective as possible. The problems get even more complicated when the person has more than two conditions.

Unfortunately, prior to the present invention, there were no known self-care systems which effectively provide single integrated systems for self-care by people with the multiple different medical conditions which the present disclosure addresses.

Prior to the present invention, these problems were not being addressed in the healthcare industry. Rather, the medical literature has been first discussing substantial problems with known clinical practice guidelines or treatment protocols. Clinical practice guidelines and treatment protocols are substantially different than self-care systems. Clinical practice guidelines and treatment protocols are standardized guidelines for health care professionals (such as doctors and nurses) to follow when they are treating patients. These clinical practice guidelines and treatment protocols are not intended to be and are not written for patients to follow for self-care and generally are not readily understandable or usable by people other than medically trained professionals.

Milliman Inc., and McKesson Corp. are two nationally recognized companies that are currently distributing clinical practice guidelines and treatment protocols for healthcare professionals (such as doctors and nurses). Every hospital in the United States must adopt one of these two sets of protocols to receive accreditation. These protocols are only for use in hospitals and only for use by healthcare professionals.

The medical literature has been discussing substantial problems with such types of known clinical practice guidelines or treatment protocols. For instance, the Aug. 10, 2005, article entitled “Clinical Practice Guidelines and Quality of Care for Older Patients with Multiple Co-morbid Diseases: Implications for Pay-for-Performances” by Darer J. Boyd CM, et al. published by JAMA provides that:
CONTEXT: Clinical practice guidelines (CPGs) [for doctors to follow] have been developed to improve the quality of health care for many chronic conditions. Pay-for-performance initiatives assess physician adherence to interventions that may reflect CPG recommendations.
OBJECTIVE: To evaluate the applicability of CPGs to the care of older individuals with several comorbid diseases.
DATA SOURCES: The National Health Interview Survey and a nationally representative sample of Medicare beneficiaries (to identify the most prevalent chronic diseases in this population); the National Guideline Clearinghouse (for locating evidence-based CPGs for each chronic disease).
STUDY SELECTION: Of the 15 most common chronic diseases, we have selected hypertension, chronic heart failure, stable angina, atrial fibrillation, hypercholesterolemia, diabetes mellitus, osteoarthritis, chronic obstructive pulmonary disease, and osteoporosis, which are usually managed in primary care, choosing CPGs promulgated by national and international medical organizations for each.
DATA EXTRACTION: Two investigators independently assessed whether each CPG addressed older patents with multiple comorbid diseases, goals of treatment, interactions between recommendations, burden to patients and caregivers, patent preferences, life expectancy, and quality of life. Differences were resolved by consensus. For a hypothetical 79-year-old woman with chronic obstructive pulmonary disease, type 2 diabetes, osteoporosis, hypertension, and osteoarthritis, we aggregated the recommendations from the relevant CPGs.
DATA SYNTHESIS: Most CPG's did not modify or discuss the applicability of their recommendations for older patients with multiple comorbidities. Most also did not comment on burden, short- and long-term goals, and the quality of the underlying scientific evidence, nor give guidance for incorporating patient preferences into treatment plans. If the relevant CPGs were followed, the hypothetical patient would be prescribed 12 medications (costing her 406 dollars per month) and a complicated nonpharmacological regimen. Adverse interactions between drugs and diseases could result.
CONCLUSIONS: This review suggests that adhering to current CPGs in caring for an older person with several comorbidities may have undesirable effects. Basing standards for quality of care and pay for performance on existing CPGs could lead to inappropriate judgment of the care provided to older individuals with complex comorbidities and could create perverse incentives that emphasize the wrong aspects of care for this population and diminish the quality of their care. Developing measures of the quality of the care needed by older patients with complex comorbidities is critical to improving their care.

The Aug. 4, 2005 article, entitled “Following Clinical Practice Guidelines for Older Adults With Several Illnesses” which discusses the JAMA study further explains that:
Current clinical practice guidelines [for doctors to follow] are not written with older adults with multiple illnesses in mind, according to a study in the August 10 issue of JAMA.
The aging of the population and the increasing prevalence of chronic diseases pose challenges to the development and application of clinical practice guidelines (CPGs), according to background information in the article. In 1999, 48 percent of Medicare beneficiaries aged 65 years or older had at least 3 chronic medical conditions and 21 percent had 5 or more.
Clinical practice guidelines are based on clinical evidence and expert consensus to help decision making about treating specific diseases. Most CPGs address single diseases in accordance with modern medicine's focus on disease and pathophysiology. However, physicians who care for older adults with multiple diseases must strike a balance between following CPGs and adjusting recommendations for individual patients' circumstances. Difficulties escalate with the number of diseases the patient has. The limitations of current single-disease CPGs may be highlighted by the growth of pay-for-performance initiatives, which reward practitioners for providing specific elements of care. Because the specific element of care may be based on single-disease CPGs, pay-for-performance may create incentives for ignoring the complexity of multiple comorbid (co-existing illnesses) chronic diseases and dissuade clinicians from providing optimal care for individuals with multiple comorbid diseases.
Cynthia M. Boyd, M.D., M.P.H., from the Center of Aging and Health, Johns Hopkins University, Baltimore, and colleagues examined how CPGs address comorbidity in older patients and explored what happens when multiple single-disease CPGs are applied to a hypothetical 79-year-old woman with 5 common chronic diseases. Selection of these diseases were based on data from the National Heath Interview Survey and a nationally representative sample of Medicare beneficiaries (to identify the most prevalent chronic diseases in this population). The National Guideline Clearinghouse was used to locate evidence based CPGs for each chronic disease. Of the 15 most common chronic diseases, the researchers focused on CPGs for hypertension, chronic heart failure, stable angina, atrial fibrillation, hypercholesterolemia, diabetes mellitus, osteoarthritis, chronic obstructive pulmonary disease, and osteoporosis.
Two investigators independently assessed whether each CPG addressed older patients with comorbidities, goals of treatment, interactions between recommendations, burden to patients and caregivers, patent references, life expectancy, and quality of life. For a hypothetical 79-year-old woman with chronic obstructive pulmonary disease, type 2 diabetes, osteoporosis, hypertension, and osteoarthritis, the authors aggregated the recommendations from the relevant CPGs.
The researchers found that most CPGs did not modify or discuss the applicability of their recommendations for older patients with multiple comorbidities. Most also did not comment on burden, short- and long-term goals, and the quality of the underlying scientific evidence, nor give guidance for incorporating patients preferences into treatment plans. If the relevant CPGs were followed, the hypothetical patient would be prescribed 12 medications (costing her $406 per month) and a complicated nonpharmacological regimen. Adverse interactions between drugs and diseases could result.
“For the present, widely used CPGs offer little guidance to clinicians caring for older patents with several chronic diseases. The use of CPGs as the basis for pay-for-performance initiatives that focus on specific treatments for single diseases may be particularly unsuited to the care of older individuals with multiple chronic diseases. Quality improvement and pay-for-performance initiatives within the Medicare system should be designed to improve the quality of care for older patients with multiple chronic diseases; a critical first step is research to define measures of the quality of care needed by this population, including care coordination, education, empowerment for self-management and shared decision making based on the individual circumstances of older patients,” the authors conclude. (JAMA. 2005; 294:716-724. http://www.jamamedia.org.)

In an accompanying editorial, Patrick J. O'Connor, M.D., M.P.H., of the HealthPartners Research Foundation, Minneapolis, commented on the JAMA study by Boyd et al. as follows:
Despite their limitations, evidence-based CPGs remain an important and necessary tool in the effort to improve health care quality. Strategies to address the limitations of current CPGS need to be developed and implemented, including providing recommendations based on level of evidence for particular patient groups and considering the potential economic and personal burden on the patent and caregiver as well as potential interactions with comorbid conditions. Future CPGs could be improved by including explicit information such as the number needed to treat to obtain a specified benefit, and should also be crafted more systematically to consider the influence of patient-specific factors such as age, life expectancy, and comorbidity on anticipated benefits of interventions. In addition, CPGs could include information on cost of various potential therapies, which may influence patient preferences and patient adherence to therapeutic regimens. Such modifications will increase the value of CPGs to clinicians and patients at the point of car, especially when physicians have too much to do [in a given office visit].
Encouraging customization of care in complex clinical scenarios respects the individuality of patents and the professional judgment of highly skilled physicians and minimizes the problem of overtreating patients most susceptible to drug interactions, drug adverse effects, and medical error. Boyd and colleagues have presented these important ‘in the trenches’ issues in a clear and compelling way. Physicians and designers of CPGs owe it to themselves and their patients to consider these issues carefully and to craft CPGs and pay-for-performance accountability measures that will reinforce excellent clinical care while being mindful of resource use and being respectful of patient preferences and priorities.

The Aug. 17, 2005, article entitled “Guidelines May Fail to Meet Needs of Elderly Patients With Comorbidities” by Karia Gale from Reuters Health also addresses the JAMA study in the following manner:
Current clinical practice guidelines [for doctors to follow] are designed to manage single diseases, offering little guidance to clinicians caring for older patients who have several chronic illnesses, authors of a new study suggest.
“Following clinical practice guidelines for single diseases in patients with multiple chronic conditions is very complex and costly and may lead to adverse consequences, including polypharmacy with its associated risks of adverse effects and drug interactions and even hospitalizations.” Lead investigator Dr. Cynthia M. Boyd told Reuters Health.
This is especially pertinent, she added, because pay-for-performance incentives may be based on quality of care standards created for the management of single diseases, whereas half of patents over age 65 have three or more chronic conditions. The care of these patients accounts for almost 90% of Medicare's annual budget.
“Rewarding physicians based on what is good care for younger patients with single diseases is unrealistic,” the researcher added. “Performance incentives based on this model may penalize physicians caring thoughtfully for older patients and may impact the quality of care those patients receive.”
For their study, Dr. Boyd from Johns Hopkins University in Baltimore and her associates identified the most recently released evidence-based guidelines for hypertension, chronic heart failure, stable angina, a trial fibrillation, hypercholesterolemia, diabetes, osteoarthritis, chronic obstructive pulmonary disease (COPD), and osteoporosis.
They found that only guidelines for diabetes, chronic heart failure, angina, and hypercholesterolemia gave general-guidance for patients with several comorbid conditions. None discussed the burden of comprehensive treatment on patients or caregivers, and only the guidelines for chronic heart failure explicitly discussed end-of-life treatment.
Dr. Boyd's group used guidelines to develop a treatment plan for a hypothetical 79-year-old woman with osteoporosis, osteoarthritis, type 2 diabetes, hypertension, and COPD.
If all the recommendations were followed, the patient would require 12 separate medications taken as 19 doses at five times during a typical day. Without any insurance coverage for prescription drugs, that would amount to approximately $400 per month. If she were a typical Medicare patient, her costs with the new Medicare drug benefit would still add up to more than $3700 per year.
“We need to think less about individual disease and more about individual people who are living longer with multiple chronic conditions,” Dr. Boyd said. More research is needed, she added, to form “reasonable estimates of risks, benefits and burdens that are specific to them and their individual circumstances and preferences.”
Dr. Patick J. O'Connor, from HealthPartners Research Foundation in Minneapolis, Minn., agrees with this assessment, according to his accompanying editorial.
“Ideally,” he writes, “clinical practice guidelines would help physicians select from among multiple evidence-based recommendations those with the greatest potential benefit to a given patient.”

This medical literature and commentary clearly explains the lack of and need for integrated treatment protocols and clinical practice guidelines for healthcare professionals (such as doctors and nurses) to follow in treating patients with multiple different diseases or different medical conditions. Thus, there is clearly a need for integrated treatment protocols and clinical practice guidelines for healthcare professionals (such as doctors and nurses).

Similarly, the existing literature does not appear to suggest that the healthcare industry prior to the present invention has considered integrated protocols for patient self-care. In February, 2005, Milliman and Robertson introduced individual treatment protocols for use by healthcare professionals outside of hospitals and for individual chronic conditions. Health plans have been buying these treatment protocols to start individual disease management programs. However, these treatment protocols for healthcare professionals to follow are not the same as self-care programs that patients (rather than healthcare professionals) must follow at home and usually alone to treat themselves. These treatment protocols would be completely unusable by a person attempting provide self-care for multiple simultaneously existing medical conditions.

Accordingly, there is a substantial need for self-care systems for treating multiple simultaneously existing different medical conditions.

### SUMMARY

The present disclosure provides systems, methods and apparatus that enable a user to provide integrated self-care for blood sugar control, blood pressure control and heart failure care.

In one embodiment, the present disclosure provides an integrated blood sugar control, blood pressure control and heart failure care self care system. In one embodiment, the self-care system includes (a) a personal health folder (b) a process guide, (c) an education guide, and (d) various programs specifically designed to address these multiple health conditions of the user. In one embodiment, the process guide includes at least one record book, one or more magnet boards and a wallet card. In one embodiment, the programs included in the system include (i) a test program, (ii) a meal program, (iii) a move or exercise program, (iv) a medication program, and (v) a general health program. In one embodiment, scales, blood pressure monitor guide and blood pressure monitor are included in the system to be used in the test program. In one embodiment, the meal program includes a food guide, a food poster, a loose food guide, a fast food guide, a portion plate, a meal planner game, a meal planner and a shopping list. In one embodiment, the move or exercise program includes a skin and foot care guide and a walking guide. In one embodiment, the move program further includes skin lotion, mirror and a foot filament guide. In one embodiment, the medication program includes a medication guide, an insulin administration guide, a pill organizer and glucose tablets. In one embodiment, the general health program includes a tobacco addiction guide and a stress management guide.

It should be appreciated that one significant benefit of the present disclosure is the increased likelihood that the patient will actually read and use this single set of materials which is substantially less volume than multiple sets of materials for each condition. When a chronic care program delivers multiple sets of materials, the sheer amount often discourages the person from trying at all. The more conditions a person has, the more likely the person will be overwhelmed. In the combination of any disease with Heart Failure, the mere name of the disease can cause a person to give up trying to manage their health. Even if they do read through everything, they will not learn the most important issue of having such multiple diseases; that is, a person who adds Hypertension to their Heart Failure is more likely to have more episodes of the heart pumping ineffectively than a person who has heart failure alone. A person who adds both Hypertension and Diabetes to Heart Failure is even more likely to suffer frequent episodes than a person who has heart failure and just one of the other diseases. None of the three sets of independent materials will alert the person to their heightened risk.

The present disclosure avoids another danger that the person will read one set and put the others away for later, often never coming back to them. For instance, if the person reads Hypertension first, they will seek to avoid salt, but the material is not likely to alert them to problems with potassium. The heart functions by alternating activities of sodium and potassium, but potassium is not a concern until a person's health has seriously deteriorated. Hypertension is such a common disease that individual instructions do not assume that the person has advanced, serious co-existing conditions. The person with hypertension and heart failure must ask their doctor whether they need a diet high or low in potassium, whether to seek or avoid potassium-containing foods, and whether to take a potassium supplement.

It is most likely, if any material is skipped or “put away for later,” it will be the Diabetes materials. A significant percentage of people with diabetes are in denial about their disease and will deliberately avoid materials that are labeled “Diabetes.” In this way, they miss crucial information about controlling carbohydrates in their diet.

By integrating the three sets of instructions about diet, the present disclosure enables (and to certain extent forces) the person to see the entire picture at once.

Of the three most important lifestyle changes for such chronic diseases and the most difficult to manage is diet. With three chronic diseases, three sets of instructions, the challenge can be daunting, even with sincere effort. For example, all other educational program's individual sets of instructions attempting to teach how to read Nutritional Facts labels, teach all of the lines of information. The present system teaches just the essential lines for the particular disease. If a person only has attend to a single set of essential data from the entire label, the person is more likely to sustain attention. However, if all three sets teach the entire Nutritional Facts label, it will be much more difficult to extract what is essential (i.e., a person may miss that there is something different to learn about each one). They may see a similar section begin in a new set of materials, assume they already know what is there, and skip it. The present system teaches what is essential for all three diseases combined using a single system.

For one embodiment of the present triple-condition system or kit, the diet section can be integrated and radically simplified with a card game for learning meal planning. In one such embodiment, the suits are the food groups, each card represents a serving of a particular food, and the essential nutritional values of salt, cholesterol and carbs are the number values on the card. The goal is to plan a day's meals without going over the limits for salt, cholesterol and carbs. By playing the game, a person learns what combinations of foods will work together for a successful day's diet plan. The delight and familiarity of a card game increases the likelihood that the person will both attempt the challenge of meal planning and the likelihood that they will understand and follow the meal planning instructions.

A further benefit of the integrated approach disclosed herein is that the basic understanding of the multiple diseases is simplified and coherent; that is, both Heart Failure and Hypertension mean the heart is not pumping blood adequately through the body, but for different reasons. Education about Heart Failure typically emphasizes the excess amount of fluid. People with Hypertension can be assumed to have cholesterol sticking to arteries and clogging the pathways. Education about Diabetes alone will fail to explain the impact of Diabetes on the heart disease. Extra sugar in the blood makes everything more sticky and thick, compounding the difficulty of pumping blood throughout the body. That simple, integrated statement can be a breakthrough understanding for a patient and will not be found in three separate sets of material. Thus, the present disclosure not only provides one integrated set of materials, but also an integrated understanding of how the three conditions relate to one another. The disclosure may reconcile what may be contradictions and highlight what others may miss.

Additional features and advantages of the present system are described in, and will be apparent from, the following Detailed Description and the figures.

### BRIEF DESCRIPTION OF THE DRAWINGS

FIG. 1A is a diagram illustrating the relationship between the components of an embodiment of the disclosed integrated system.

FIG. 1B is a front view of components included in an embodiment of the disclosed integrated system.

FIGS. 2A, 2B, 2C and 2D are front views of a process guide included in an embodiment of the disclosed integrated system.

FIGS. 3A, 3B, 3C, 3D, 3E, 3F, 3G and 3H are front views of an education guide included in an embodiment of the disclosed integrated system.

FIGS. 4A, 4B, 4C, 4D, 4E, 4F, 4G, 4H, 4I, 4J and 4K are front views of a record book included in an embodiment of the disclosed integrated system.

FIG. 5 is a front view of a magnetic board of one embodiment of the present disclosure.

FIGS. 6A and 6B is a front view of a wallet card of one embodiment of the present disclosure.

FIGS. 7A, 7B, 7C, 7D, 7E, 7F, 7G and 7H is a front view of a food guide included in an embodiment of the disclosed integrated system.

FIG. 8 is a front view of a food poster included in an embodiment of the disclosed integrated system.

FIG. 9 is a front view of a portion plate included in an embodiment of the disclosed integrated system.

FIGS. 10A and 10B are front views of a loose food guide included in an embodiment of the disclosed integrated system.

FIGS. 11A, 11B, 11C and 11D are front views of a fast food guide included in an embodiment of the disclosed integrated system.

FIGS. 12A, 12B and 12C are front views of a food selection game included in an embodiment of the disclosed integrated system.

FIG. 13 is a front view of a sample meal planner included in an embodiment of the disclosed integrated system.

FIGS. 14A and 14B is a front view of a shopping list included in an embodiment of the disclosed integrated system.

FIGS. 15A and 15B are front views of a walking guide included in an embodiment of the disclosed integrated system.

FIGS. 16A and 16B are front views of a skin and foot care guide included in an embodiment of the disclosed integrated system.

FIGS. 17A, 17B, 17C, 17D, 17E, 17F and 17G are front views of a medication guide included in an embodiment of the disclosed integrated system.

FIGS. 18A, 18B, 18C and 18D are front views of an insulin administration guide included in an embodiment of the disclosed integrated system.

FIGS. 19A, 19B, 19C and 19D are front views of an automatic blood pressure monitor guide included in an embodiment of the disclosed integrated system.

FIGS. 20A and 20B are front views of a tobacco addiction guide included in an embodiment of the disclosed integrated system.

FIGS. 21A and 21B are front views of a stress management guide included in an embodiment of the disclosed integrated system.

### DETAILED DESCRIPTION OF EXEMPLARY EMBODIMENTS

The system and method described herein provide for integrated blood sugar control, blood pressure control and heart failure self-care of a single person by that person. It should also be appreciated that the system and method of the present disclosure can be employed by another person such as a care-giver, an in-home care provider, a health-care provider for the person being cared for. The disclosed method and system includes a collection of devices and instructions a user may use to simultaneously provide blood sugar control, blood pressure control and heart failure care in an integrated manner. Many of the instructions are in the form of step-by-step guides with illustrations to guide the user through use of the system and method.

It should be appreciated that the different embodiments of the present invention may include: (a) less than all of the components described below, (b) more than the components described below, or (c) one or more substitute or alternative components for one or more of the components described below.

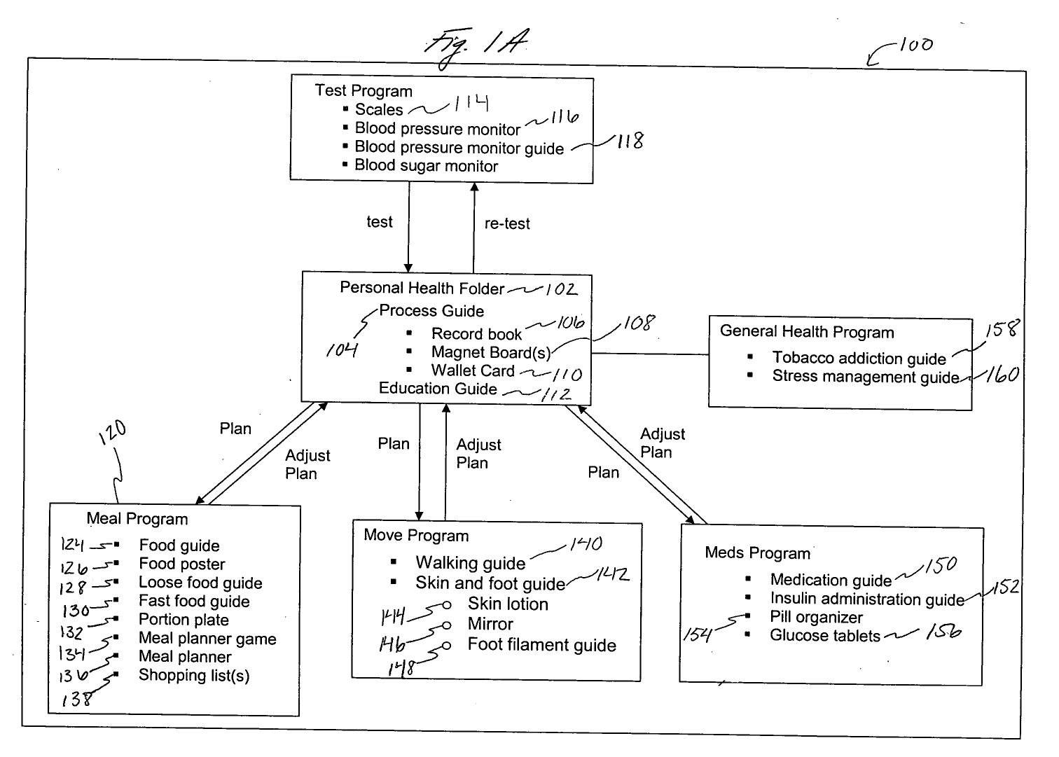

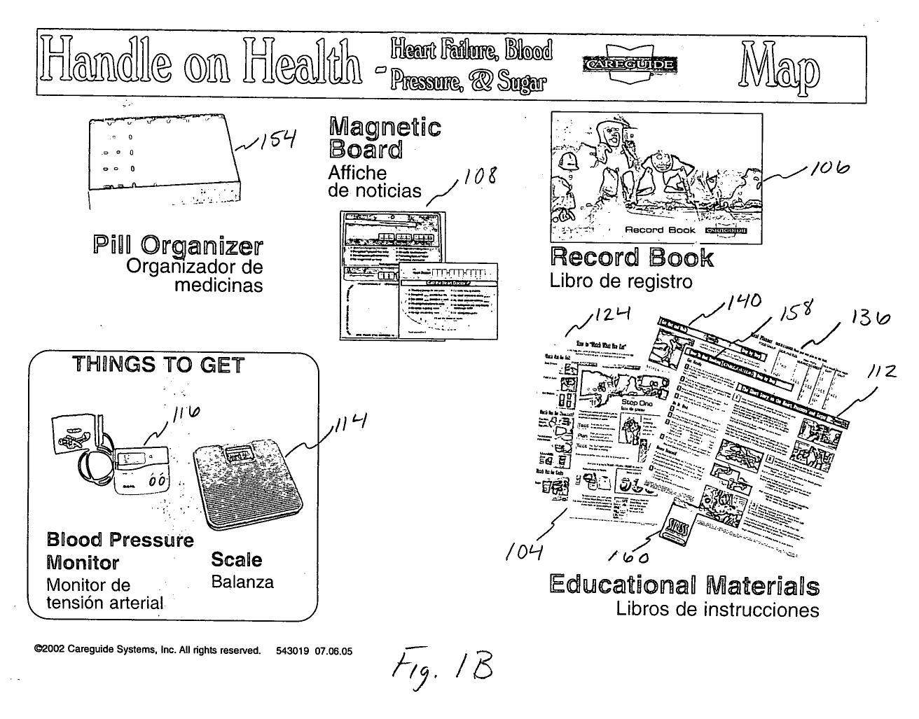

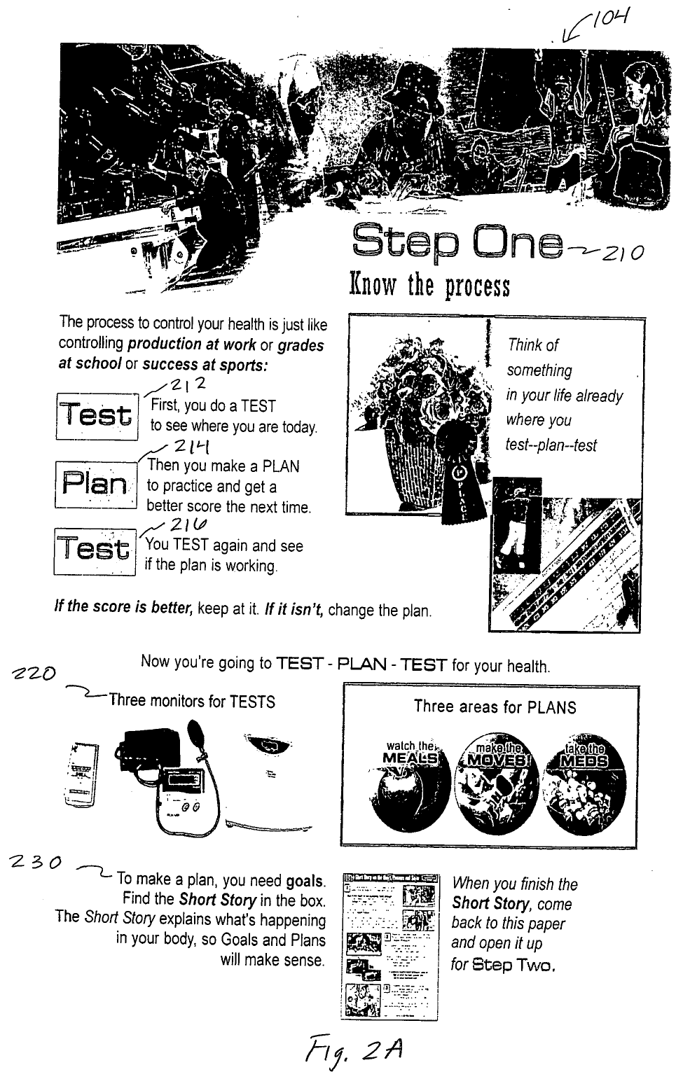

One embodiment of the system of the present disclosure is a system 100 of integrated components illustrated in FIGS. 1A and 1B. The illustrated integrated components system 100 includes a personal health folder 102, a primary step-by-step process or preparation guide 104 including a medical record keeping book 106, magnet boards 108 and wallet card 110, an education guide 112 discussing blood pressure, blood sugar and the heart, testing tools including scales 114, blood pressure monitor 116 and a blood pressure monitor guide 118 and secondary step-by-step program guides 120. The secondary step-by-step program guides 120 include a meal program including a food guide 124, a food poster 126, a loose food guide 128, a fast food guide 130, a portion plate 132, a meal planner game 134, meal planners 136 and shopping lists 138, a move program including a walking guide 140 and skin and foot guide 142, skin lotion 144, mirror 146 and foot filament guide 148, a medication program including a medication guide 150, an insulin administration guide 152, a pill organizer 154 and glucose tablets 156, and a general health program including a tobacco addiction guide 158 and stress management guide 160. Preferably, all of these items are packaged together in a suitable container such as a cardboard box. FIG. 1B illustrates examples of some of the components of the integrated system 100.

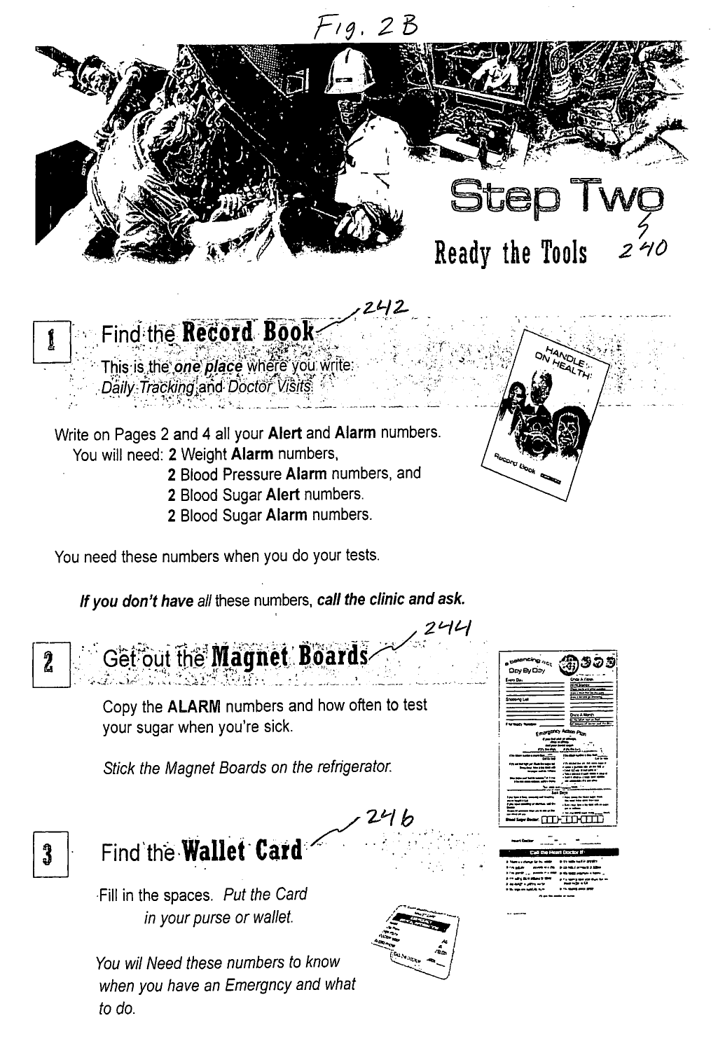

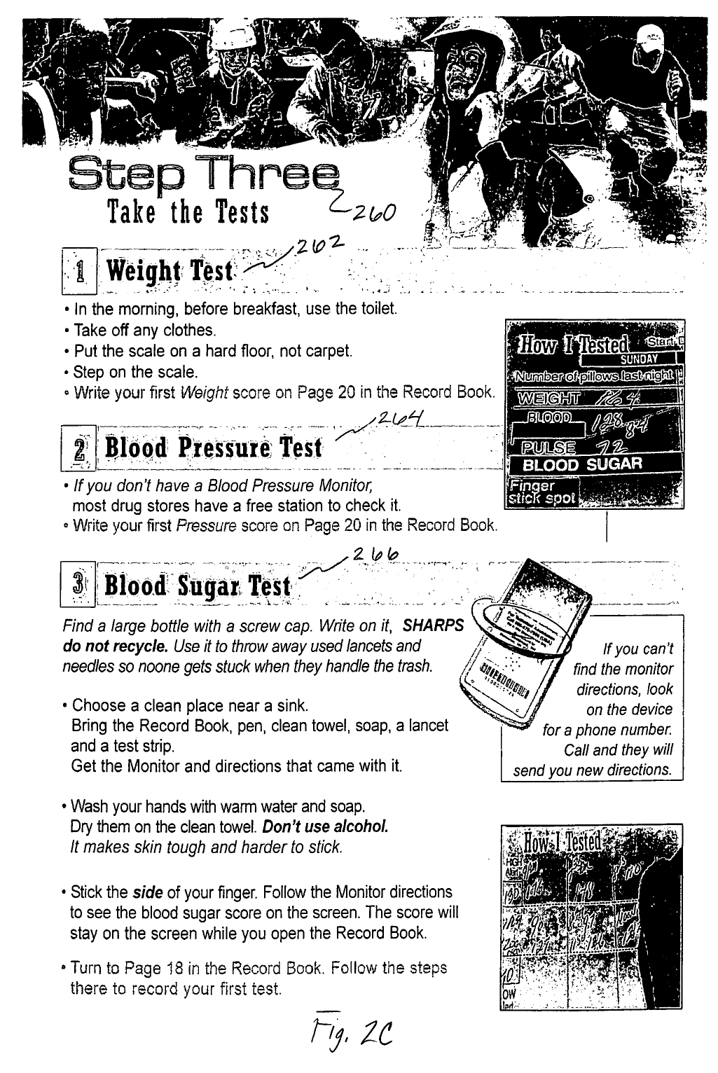

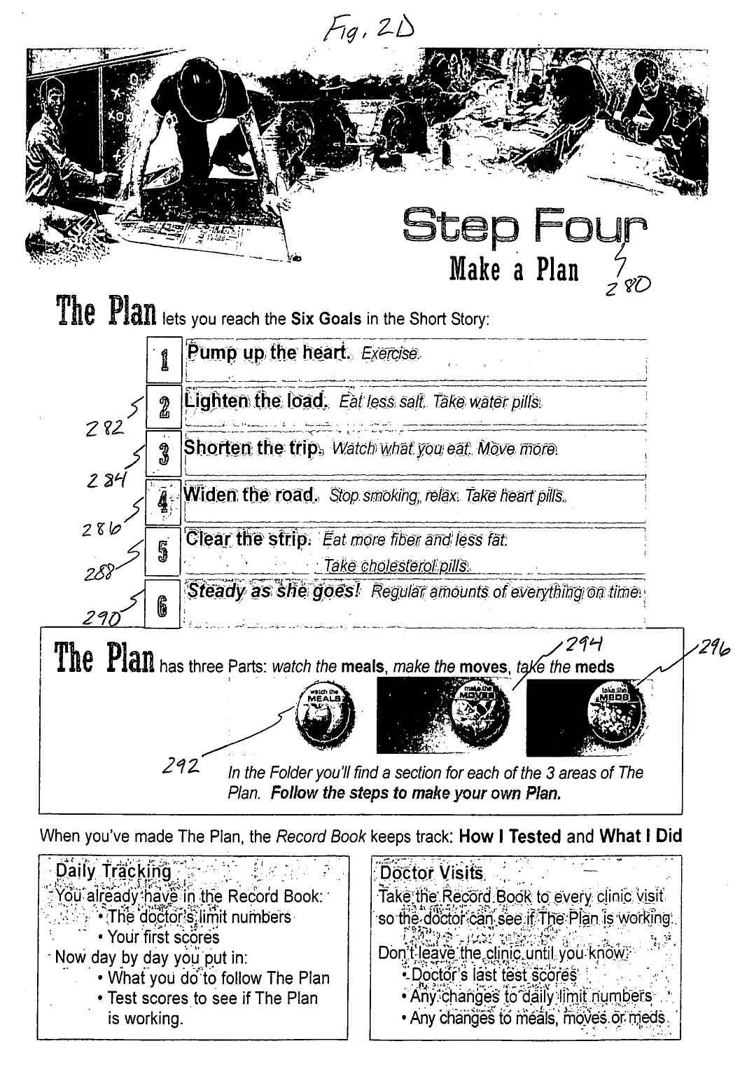

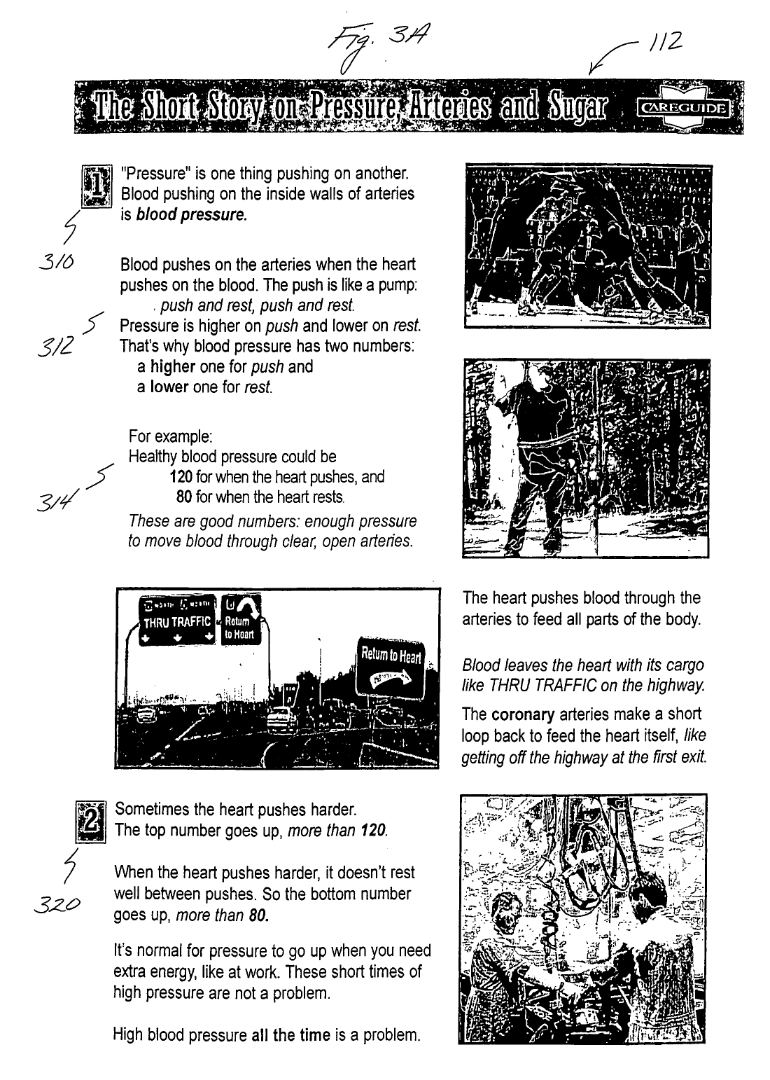

The self-care system 100 includes a personal health folder 102 to hold the record books, program guides and other materials components of the system 100; and a four-step preparation guide 104 illustrated in FIGS. 2A to 2D. Step one 210 of the preparation guide 104 is to know the process to control your health, step two 240 is to get the tools ready, step three 260 is to take the tests and step four 280 is to make a plan.

In step one 210, the preparation guide 104 presents the process of performing an assessment test 212 to determine the current condition of the user, making a plan 214 to improve the test score, and performing a follow-up test 216 to reassess whether the plan is working or needs to be changed. The preparation guide illustrates three monitors 220 used to test the health of the user. In the illustrated embodiment, a glucometer or blood sugar monitor, a blood pressure monitor and scales are illustrated. The monitors may or may not be included with the system 100.

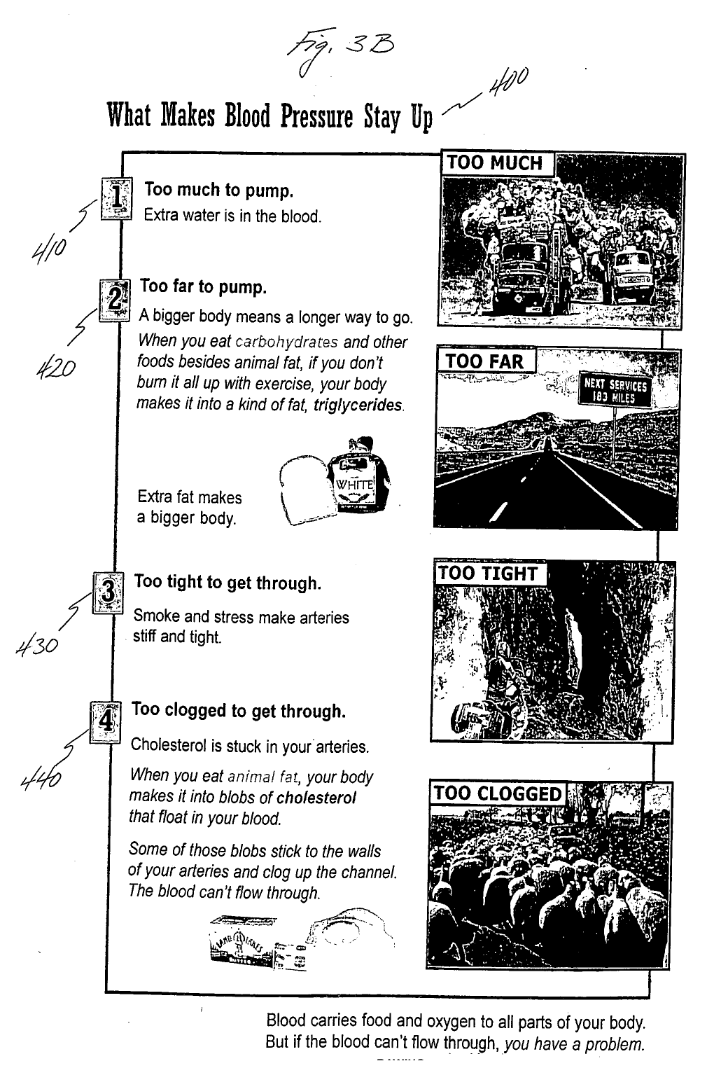

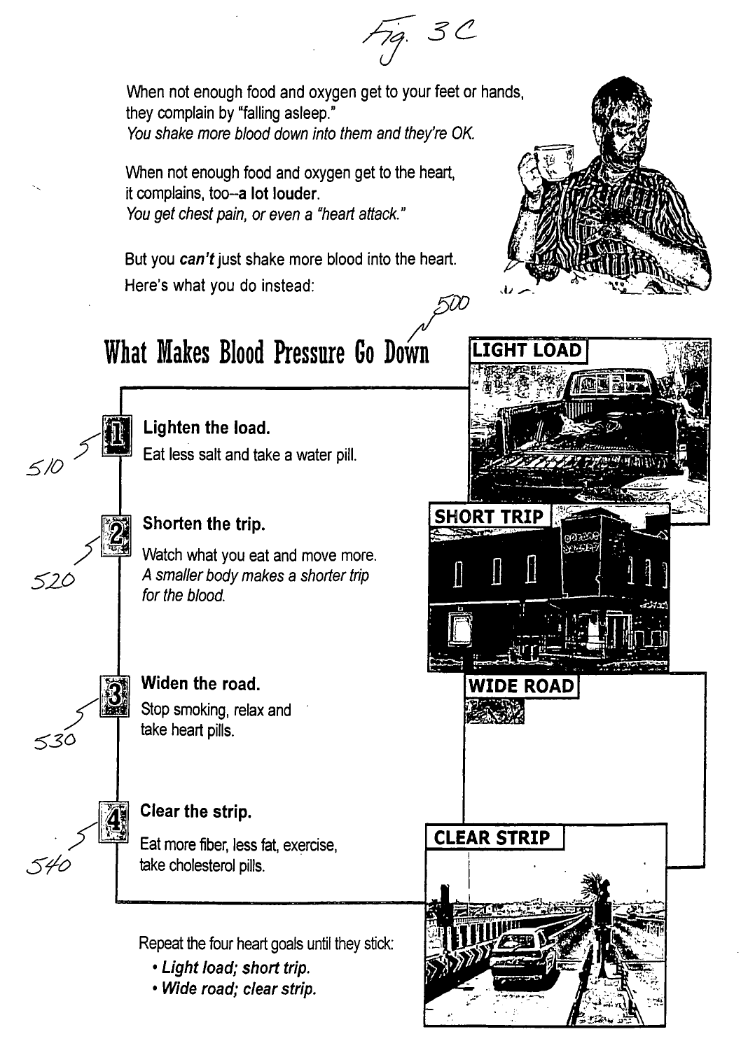

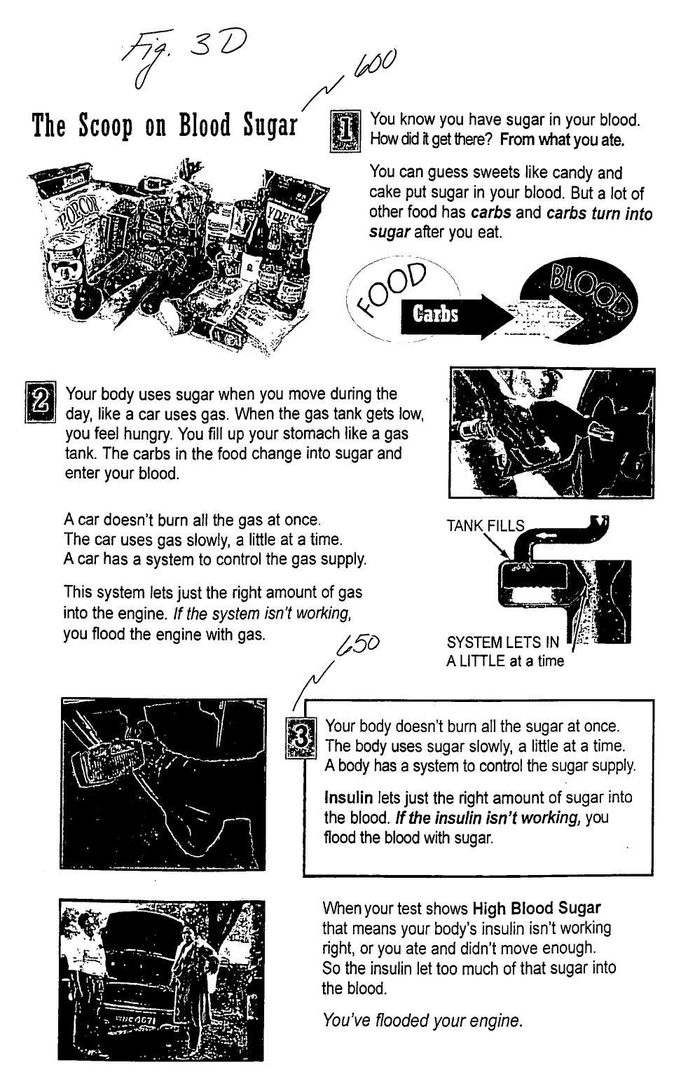

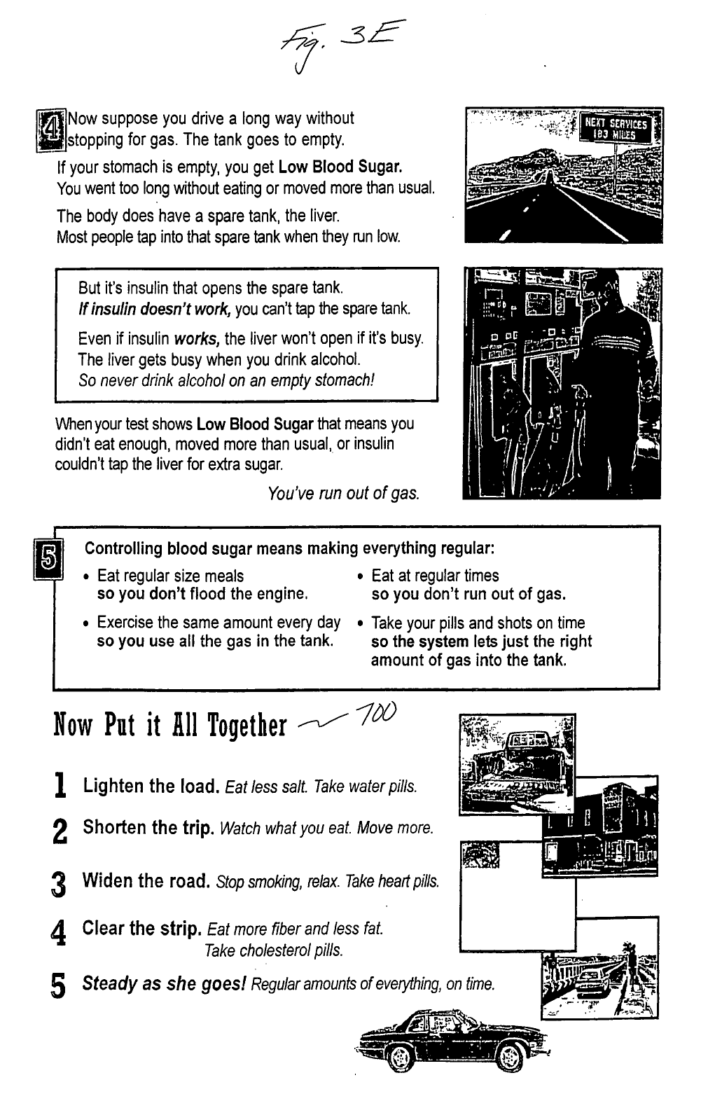

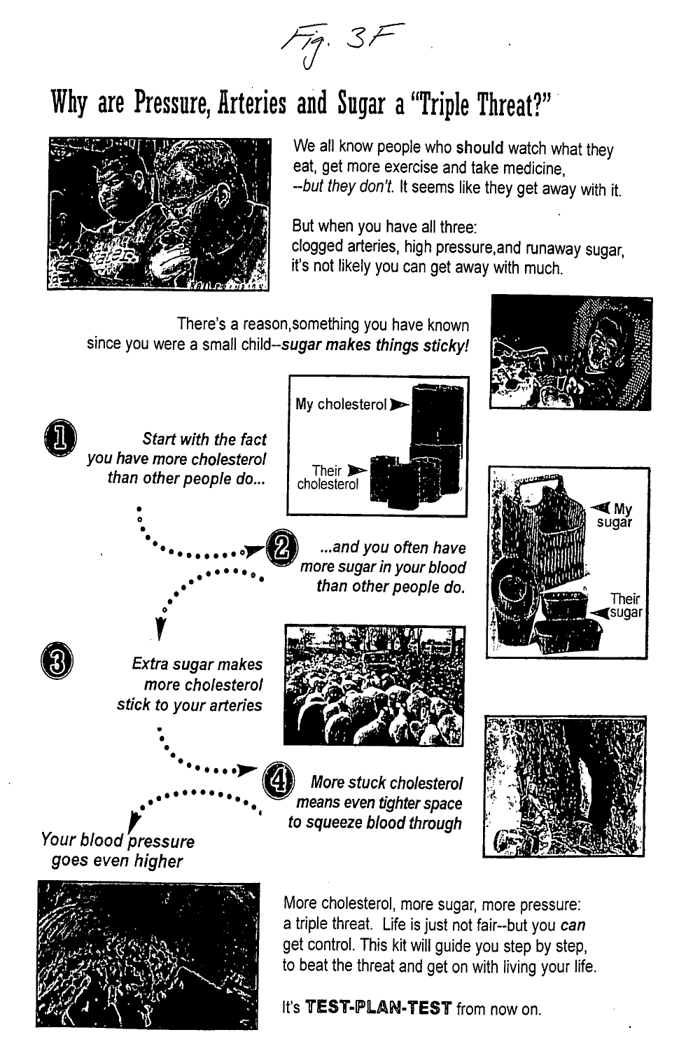

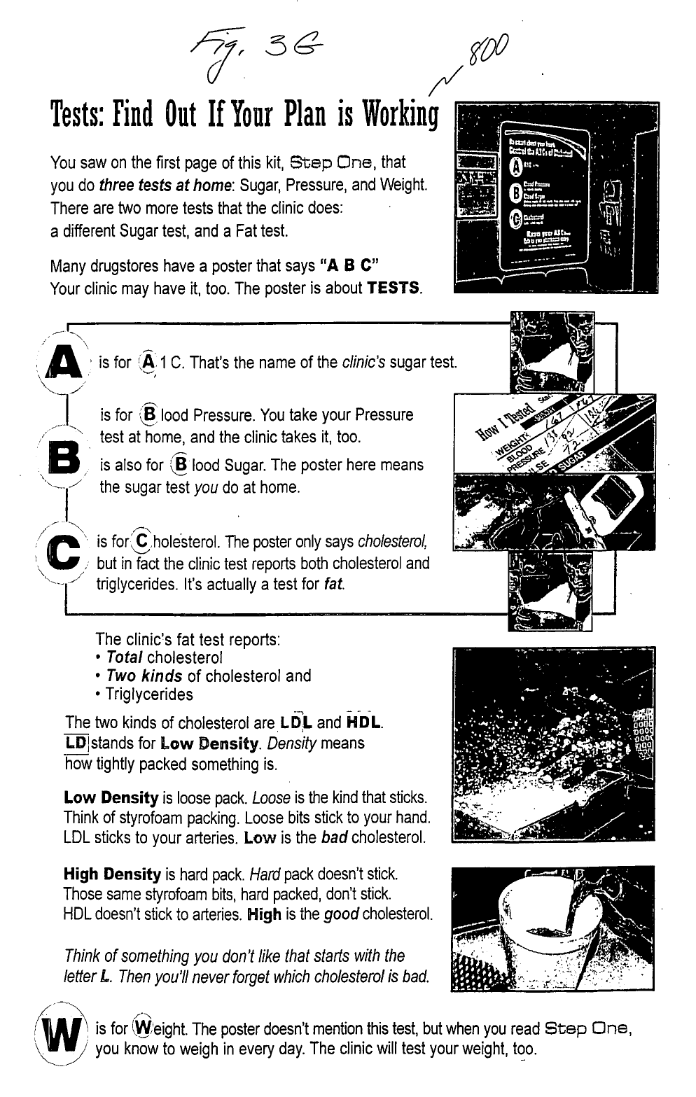

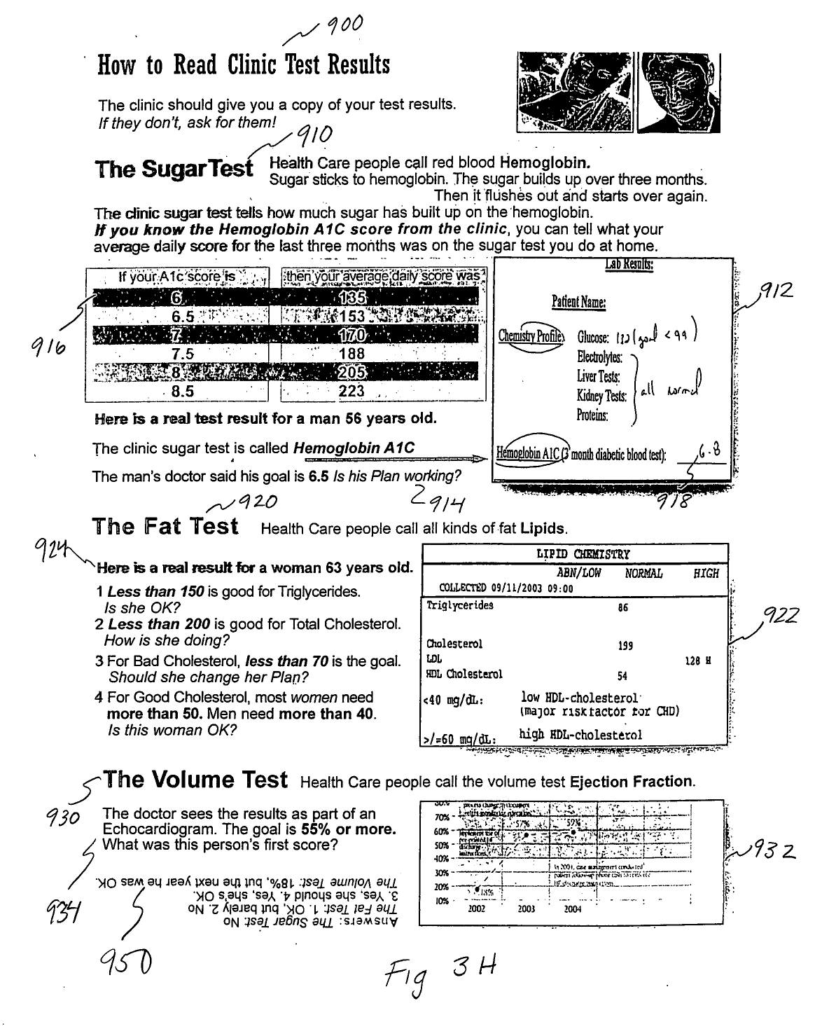

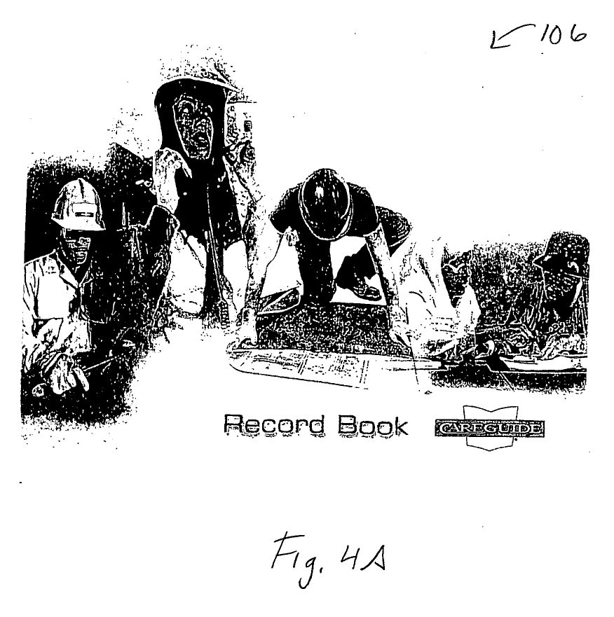

Step one 210 goes on to instruct the user to read the education guide 112 included in the system. More specifically, for the purpose of educating the user about the heart, blood pressure and blood sugar, the system 100 includes an education information guide 112 on the heart, blood pressure and blood sugar illustrated in FIGS. 3A to 3H.

The education guide 112 is entitled “The Short Story on the Heart, Pressure and Sugar.” As illustrated in FIGS. 3A to 3C, the education guide 112 includes a description of the function of the heart and what constitutes heart failure 300. The education guide also includes a definition of blood pressure 310, an explanation of why blood pressure is measured with two numbers 312 and what the two numbers represent 314, an explanation of high blood pressure 320, an explanation of what makes blood pressure go up 400, and an explanation of what makes blood pressure go down 500. In FIG. 3D, the education guide 112 further provides information about the presence and function of sugar in the blood 600, and the role of insulin and other factors that affect the amount of sugar in the blood 650. In addition, in FIGS. 3E and 3F, the education guide provides an overview of the relationship between blood sugar, blood pressure and heart failure 700.

As shown in FIG. 3G, the education guide 112 provides an explanation for the different tests required in the test-plan-test process 800 described in the preparation guide 104 including the blood sugar test, blood pressure test, cholesterol, weight, and volume. The education guide 112 explains that certain tests can be performed by the user and by health care professionals. Tests such as blood sugar, blood pressure, and weight can be performed by the user and by health care professionals.

As shown in FIG. 3H, the education guide 112 further explains that other tests are typically performed by health care professionals but provides instructions on how to read those test results 900. Certain tests, such as a sugar test 910 such as hemoglobin A1C test, a fat or lipids test 920 and a volume test 930 are typically performed only by health care professionals. In addition, the education guide 112 illustrates example lab reports 914, 924 and 934 and asks the user one or more questions 912, 922 and 932 related to the reading of the lab reports 912, 922 and 932 providing answers 950 to those questions 914, 924 and 934 in the education guide 112. In particular, FIG. 3H also illustrates an example of a lab report 912 providing a hemoglobin A1C score 918. As described in the education guide 112, the hemoglobin A1C score is a measure of how much sugar has built up on hemoglobin indicating the average daily blood sugar score. To this end, a table 916 is provided setting forth the relationship between a hemoglobin A1C score and an average daily blood sugar score.

FIG. 3H also illustrates an example of a lab report 922 providing a lipid chemistry profile. The fat test 920 measures cholesterol and triglyceride levels in the blood. Referring back to FIG. 3G, in the explanation of what cholesterol tests mean 804, the education guide 114 explains that there are two types of cholesterol. A first type of cholesterol is low density cholesterol (i.e., LDL). According to the education guide 112, low density cholesterol sticks to the arteries. This type of cholesterol is unhealthy and may need to be reduced. A second type of cholesterol is high density cholesterol (i.e., HDL). The education guide 112 explains that high density cholesterol is tough bits of hard fat that does not stick to the arteries and that this type of cholesterol is healthy and may need to be increased.

As shown in FIG. 3H further illustrates an example of a lab report 932 providing an ejection fraction graph. Ejection fraction is another test performed by health care professionals to determine the volume of blood that is pumped through the heart on each beat.

The education guide 112 attempts to make the explanation of complicated physiological concepts simple enough for one without an intimate knowledge of medical science to understand the concepts. For example, in the definition of pressure 310, mentioned above, the educational guide 112 defines “pressure” as one thing pushing on another thing and “blood pressure” as blood pushing on the inside walls of the arteries. The educational guide goes on to explain in simple terms that pressure is higher during a push state and lower during a rest state. Blood pressure is typically measured during both states. Therefore, two different numbers are measured. For example, a blood pressure measurement of 120 during the push state and 80 during the rest state is typically considered a healthy blood pressure. These and other simple explanations in the system 100 enable users to understand their multiple conditions enough to motivate the user to follow the steps to help themselves. The instructions provided in the system also explain to users what they need to do without individually addressing each separate medical condition.

Turning to FIG. 3B, in the example explanation of what makes blood pressure go up 400, the education guide 112 explains that chronic high blood pressure may be caused by extra water in the bloodstream 410 (i.e., too much to pump), excess body fat 420 (i.e., too far to pump), constricted arteries 430 (i.e., too narrow to get through), and/or block arteries 440 (i.e., too clogged to get through). The education guide 112 goes on to explain that any carbohydrates not burned up by exercise are made into a form of fat known as triglycerides. The education guide also explains that animal fat is made into cholesterol by the body which can stick to the walls of arteries to clog the channel. Pictures are included to illustrate each of the concepts taught in the educational guide 114.

Turning to FIG. 3C, in the example explanation of what makes blood pressure go down 500, the education guide explains that each of the four things described that cause blood pressure to increase can be addressed in a different way to cause blood pressure to go back down. To decrease extra water in the bloodstream 510 (i.e., lighten the load—less to pump), a person may reduce salt intake and/or take medications that reduce water in the body. To decrease excess body fat 520 (i.e., shorten the trip—less distance to pump), the person may need to lose weight by watching what they eat and moving more. To open arteries 530 (i.e., widen the road—more room to get through), the person may need to stop smoking, learn to relax to reduce stress, and/or take heart medications to widen the arteries. To unblock arteries 540 (i.e., clear the strip—less clogged to get through), the person may need to eat more fiber, eat less fat, exercise, and/or take cholesterol medication.

FIG. 3D illustrates five concepts related to blood sugar. The first concept includes how sugar gets into the blood. The second concept includes how the body uses sugar. The third concept includes how insulin affects how the body uses sugar and what is meant by high blood sugar. The fourth concept includes what is meant by low blood sugar and what causes low blood sugar. The fifth concept includes what is necessary to control blood sugar.

By educating the user about basic concepts associated with heart failure, blood pressure, and blood sugar, it is believed that the user will be more likely to understand why each of the programs (discussed below) are extremely important to follow. As a result of this understanding, the user is more likely to follow each of the steps in the single integrated programs.

Returning to step two 240 of the preparation guide 104, to further prepare to initiate the system 100, the preparation guide 104 instructs the user to refer to the record book 242, magnet boards 244 and wallet card 246, each included with the system 100.

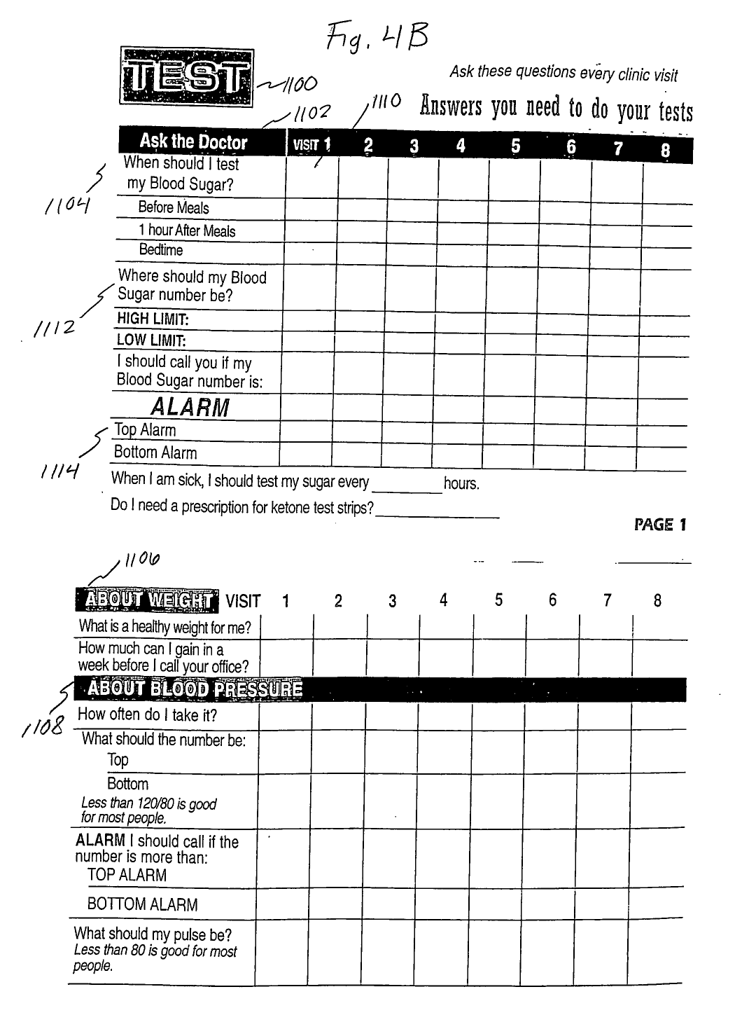

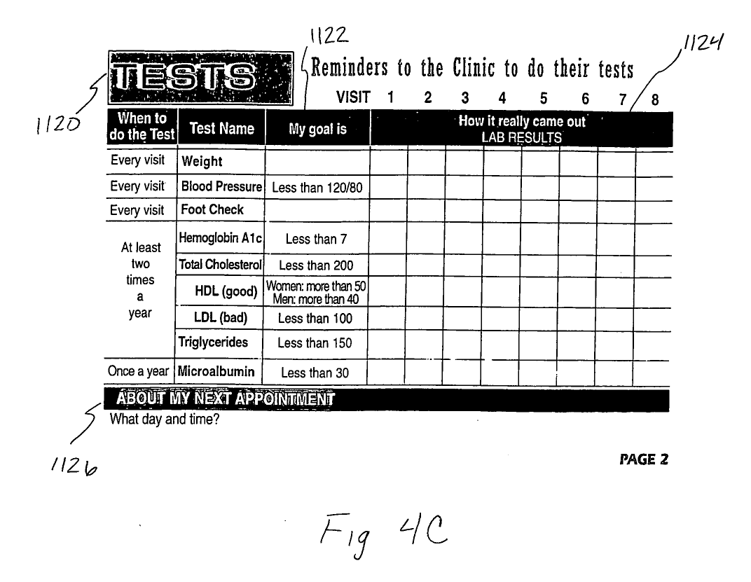

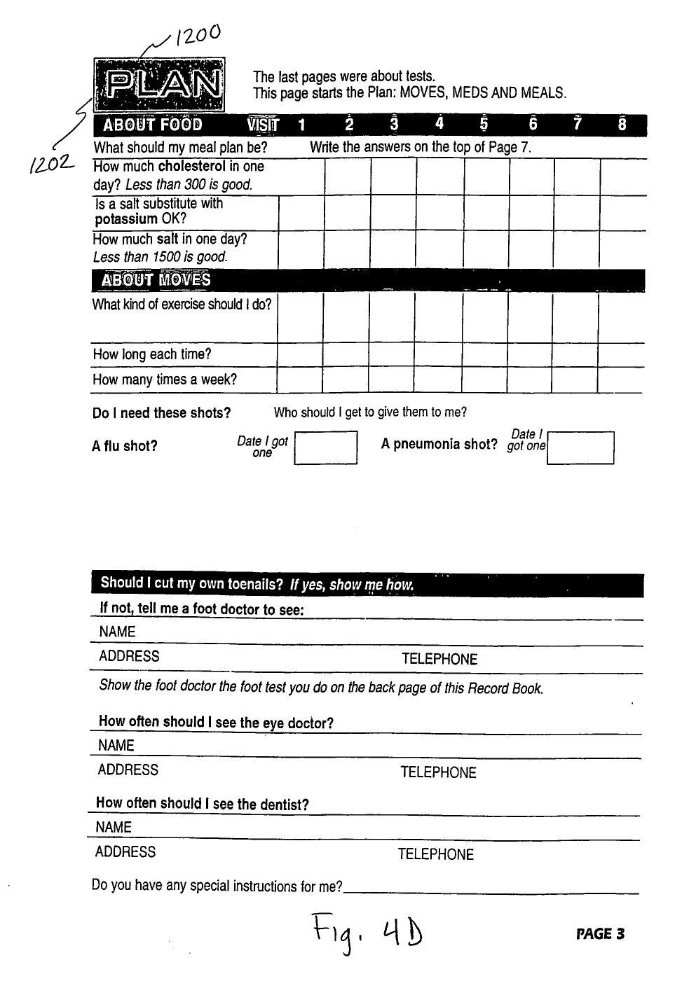

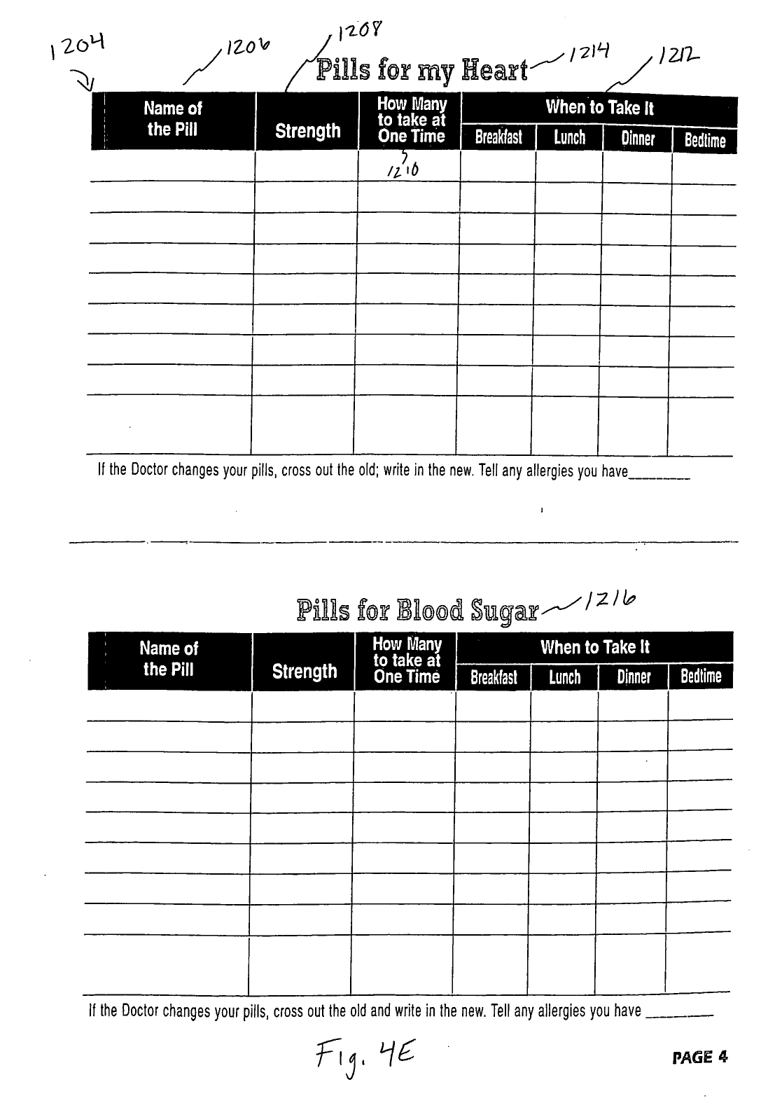

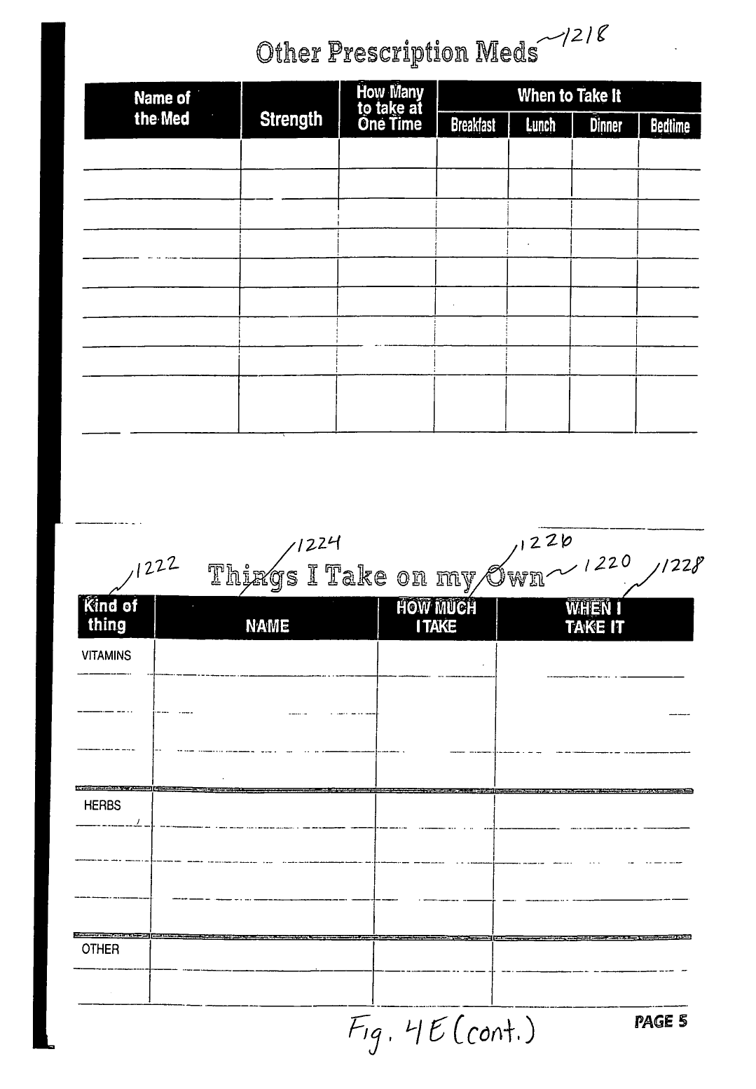

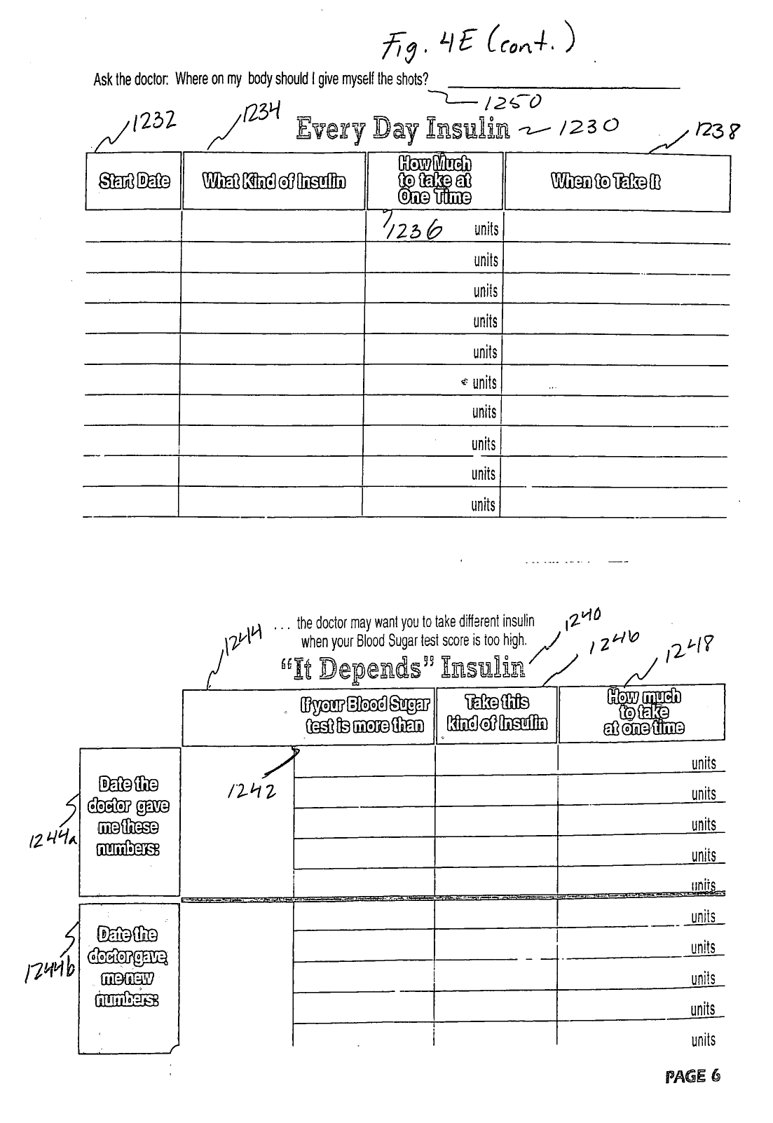

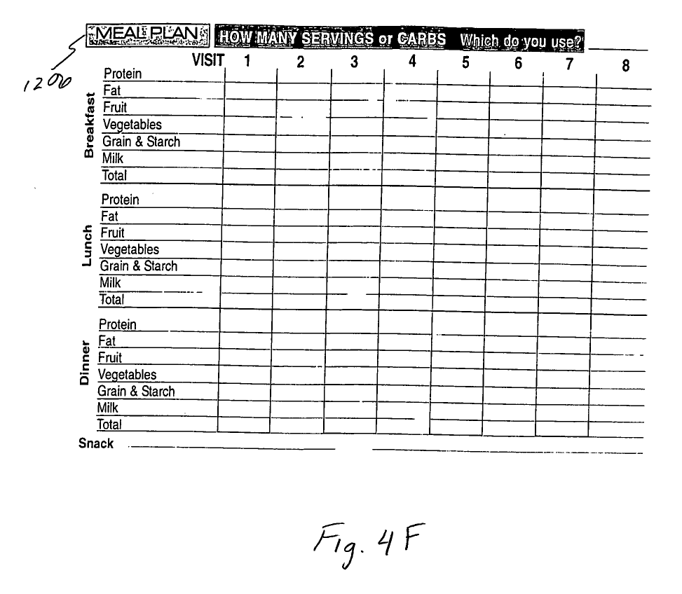

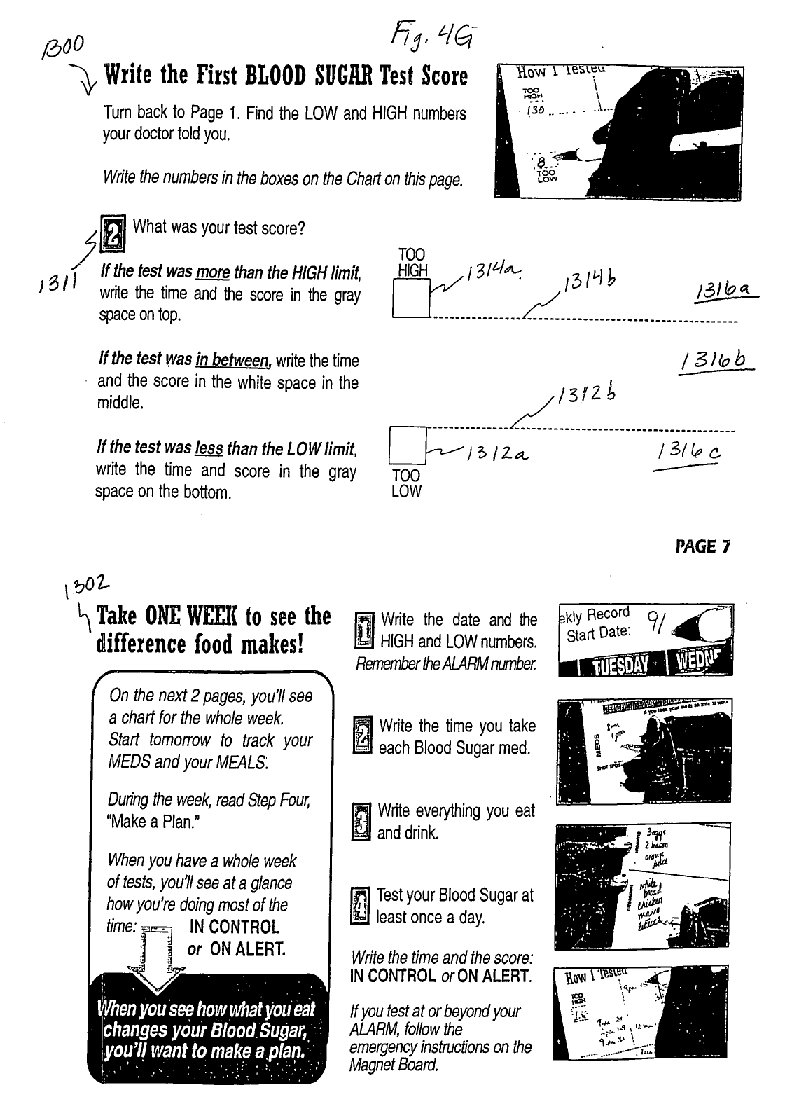

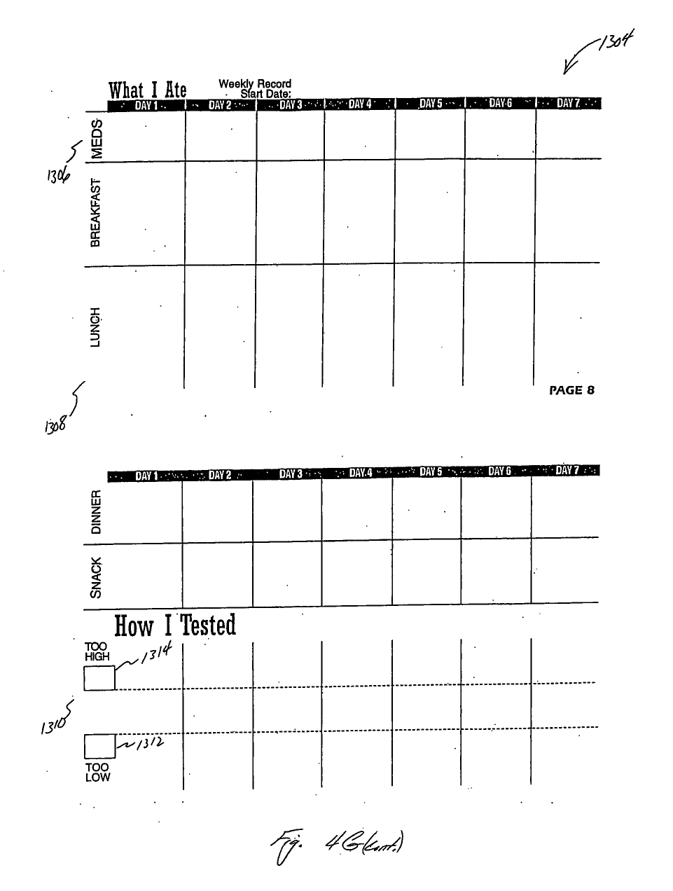

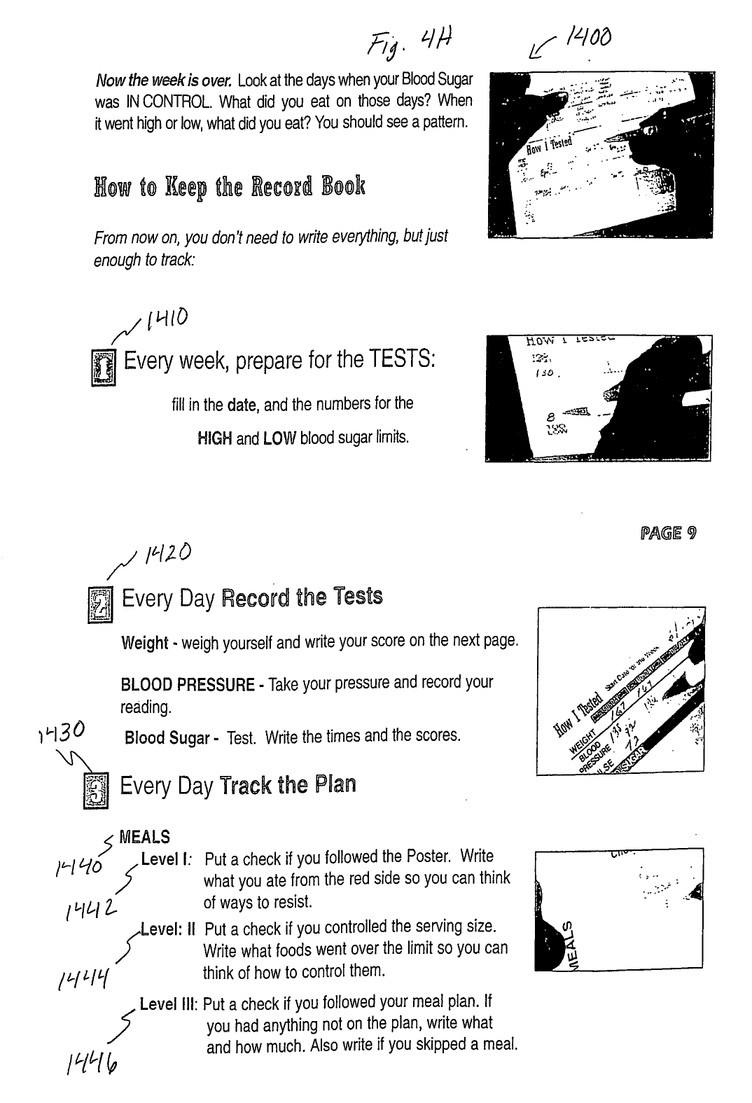

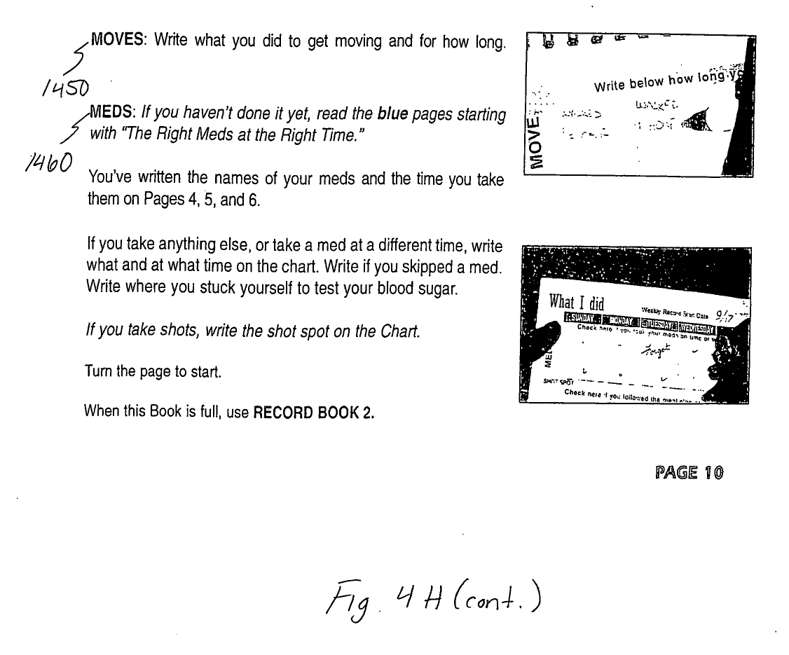

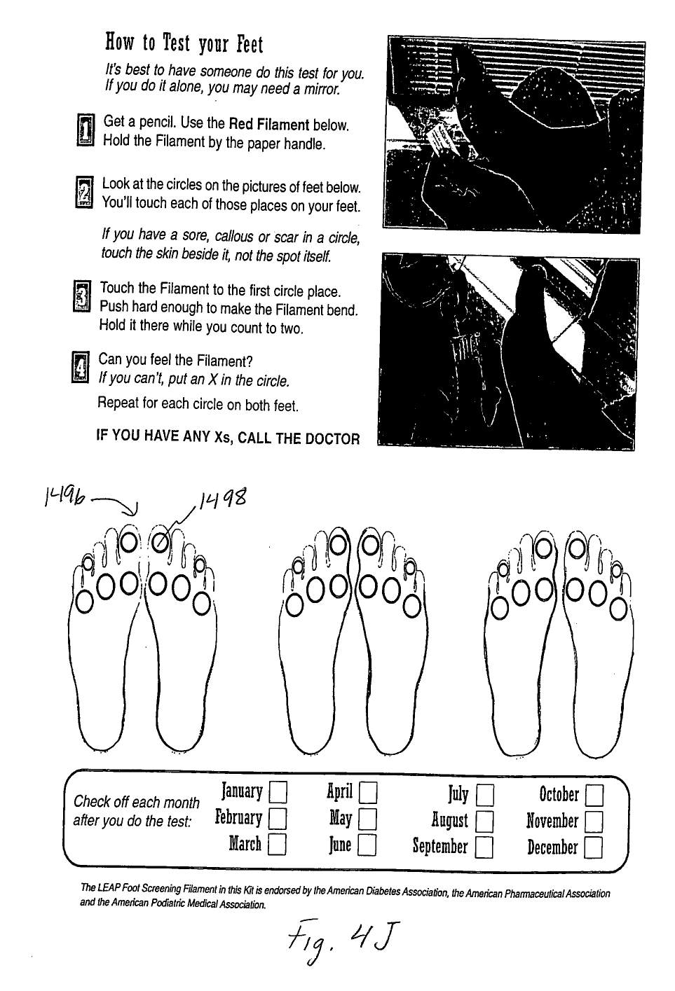

The record book 106 is illustrated in FIGS. 4A to 4K. The record book 106 includes a cover, and sections directed to the assessment tests 212, the plan 214 and the follow-up tests 216 according to the process described in step one 210 of the preparation guide 104 illustrated in FIG. 2A. In particular, as illustrated in FIGS. 4B and 4C, the test section 1100 of the record book 106 includes a place to record answers to recommended questions 1102 to be asked of the doctor on each clinic visit and a place to record blood test results 1120. The plan section 1200 of the record book 106 includes a place to record information about meals 1202, moves or exercise 1203, and medications 1204. The follow-up test section 1300 includes instructions on how to keep the medical records 1400, and a plurality of weekly record pages 1402.

The assessment tests section 1100 includes questions to ask the doctor or health care professional on each clinic visit and tests the user should expect the clinic to perform on each clinic visit. FIG. 4B illustrates a place to record answers to questions for the doctor 1102. The record book 106 includes questions about blood sugar 1104, weight 1106 and blood pressure 1108 and corresponding blanks 1110 to record the answer from the health care professional from each clinic visit. For example, the questions about blood sugar 1104 the user should ask of the health care provider include when blood sugar should be tested, what the blood sugar level should be including high and low limits of the blood sugar level 1112, what the high and low blood sugar alarm limits 1114 should be beyond which the user should call the doctor, how often the blood sugar should be checked when sick, and whether a prescription for ketone strips is needed. Questions about weight 1106 include what is a healthy weight for the user and how much weight can be gained in a week before the user would be required to notify the doctor. Questions about blood pressure 1108 include how often to take a blood pressure reading, what the blood pressure top and bottom numbers should be, at what top and bottom numbers blood pressure alarm limits should the user call the doctor, and what a pulse reading should be.

FIG. 4C illustrates a table of tests 1120. The system 100 recommends that the user remind the health care provider to perform the tests in the table 1120 on a periodic basis. Tests that are recommended to be performed on each clinic visit are weight, blood pressure, and skin and feet check. Tests recommended to be performed two times per year include a hemoglobin A1C level, and a lipid profile including total cholesterol, good cholesterol (i.e., HDL), bad cholesterol (i.e., LDL), and triglycerides. A test recommended to be performed once a year includes a microalbumin level. As illustrated in FIG. 4C, a place to record blood test results 2104 is also provided. For each of the blood test types, the record keeping book 106 includes a target score (e.g., less than 200 for total cholesterol). Questions about the next appointment 1126 may also be included such as the date and/or time of the next appointment.

The plan section 1200 of the record book 106 is illustrated in FIGS. 4D to 4F. The plan section 1200 includes a portion directed to questions to ask the doctor or health care provider 1202 and 1203 illustrated in FIG. 4D, a meds portion 1204 illustrated in FIG. 4E and a meal portion 1206 illustrated in FIG. 4F.

The first part of the plan section 1202 include questions to be asked of the health care provider about food, exercise, vaccinations, foot care, as well as referrals and frequency of visits to foot doctors, eye doctors and dentists. Questions about food may include a question on how much cholesterol and salt should be consumed each day and whether potassium is an acceptable salt substitute. Questions about exercise may include what type of exercises the user should be doing, how long the user should be exercising each time, and/or how often the user should be exercising.

FIG. 4E illustrates the med portion 1204 of the plan section 1200 of the record book 106 where a user can record information about medications. For each of the medications, the record keeping book 106 includes a blank for a name of the medication 1206, a dosage or strength of the medication 1208, the amount of medication or number of pills to take 1210, and when to take the medication 1212 (e.g., breakfast, lunch, dinner, bedtime). Preferably, the medications are grouped by the function they perform. In this example, the groups include pills for the heart 1214, pills for blood sugar 1216, other prescription meds 1218. The meds portion also includes a table to record non-prescription supplements 1220, such as vitamins and herbs, taken by the user. The table provides space for the user to record the kind of supplement 1222, name of each supplement 1224, how much of each supplement is taken 1226 and when each supplement is taken 1228.

In addition, the meds portion of the record book includes a table to record information about insulin administration. A table is provided for “every day” insulin 1230 and for “it depends” insulin 1240. The table for “every day” insulin 1230 includes a column, each for entries as to start dates 1232, what kinds of insulin is being taken 1234, how much (in units) to take at one time 1236 and when to take the insulin 1238. A separate table is provided for the “it depends” insulin 1240. “It depends” insulin may be a different insulin the doctor may want the user to take if the blood sugar test score is too high. The table 1240 provides a place to enter a blood sugar test score threshold 1242 and the date that threshold was provided by the health care provider 1244a and 1244b. The table 1240 also provides a place to enter the kind of insulin 1246 and how much (in units) of that kind of insulin should be taken by the user 1248 if the blood sugar test score of the user is more than that threshold score. Additionally, the record book provides a section 1250 to record where the doctor has instructed the user to administer the insulin.

The plan section 1200 of the record book 106 further includes a meal plan section 1206 illustrated in FIG. 4F. The meal plan section 1206 provides the user a table to record the recommended number of servings or number of carbohydrates for each food group and total for breakfast, lunch, dinner and snack(s) recommended by the health care provider. For example, the food groups may include protein, fat, fruit, vegetables, grain and starch, and milk.

The follow-up test section 1300 of the record book 106 illustrated in FIGS. 4G to 41 includes a section 1302 illustrated in FIG. 4G that enables a user to focus on a one week test period to determine whether the blood sugar is “in control” or “on alert.” In particular, the record book provides a table 1304 with spaces enabling a user to track medications 1306 and meals 1308 and their effect on blood sugar test scores 1310 on a daily basis through a period of one week. The user is instructed to record in a space provided in the record book what medications the user took and what food the user ate for breakfast, lunch, dinner and any snacks for each day of the first test week.

In addition, the user is instructed 1311 to record blood sugar test scores 1310 corresponding to each day of the test week. The blood sugar test scores are recorded in a spatial relationship to the blood sugar limits 1112 provided by the user's health care provider in the assessment tests section 1110 of the record book 106 illustrated in FIG. 4B. For example, the low blood sugar limit 1312a is indicated by line 1312b, and the high blood sugar limit 1314a is indicated by line 1314b. A blood sugar score above the high limit 1314a is recorded above line 1314b in space 1316a. A blood sugar score between the high limit 1314a and the low limit 1312a is recorded between lines 1314b and 1312b in space 1316b. A blood sugar score below the low limit 1312a is recorded below line 1312b in space 1316c.

FIG. 4H illustrates instructions on how to keep the medical records 1400 along with pictures of the section corresponding to the instructions. FIG. 4I illustrates one example of a plurality of weekly record pages 1402. The table includes columns for each day of a week and is divided into two sections: the “How I tested” section 1404 and the “What I did” section 1406.

As illustrated in FIG. 4H, the first step 1410 discussed in the record book 106 is to prepare for the tests each week by recording in the “How I tested” section 1404 the date 1408 and the numbers for the high 1312 and low 1314 blood sugar limits 1112 provided to the user by the health care provider. The second step 1420 is to record the results of the tests in the “How I tested” section 1404.

The weekly record illustrated in FIG. 4I includes a row of spaces for each test where the user may enter the test results for each day of the week. For example, the test result on Sunday for weight can be entered in space 1410; the test result on Sunday for blood pressure can be entered in spaces 1412a and 1412b; the location where the user obtained the specimen can be entered in space 1413; and the test result on Sunday for blood sugar can be entered in space 1414. Again, the user is instructed to record blood sugar test scores corresponding to each day and in spatial relation along a vertical axis to the high and low blood sugar limits provided by the health care provider as described above. In addition, the test result on Sunday for a pulse reading can be entered in space 1416.

As illustrated in FIG. 4H, the third step 1430 is to track the plan in the “What I did” section 1406 of the medical record tables illustrated in FIG. 4I, which includes spaces to enter information about the user's meals 1440, moves or exercise 1450, and medications 1460. In FIG. 4H, for meals, the user is instructed to record information at three levels. The first level 1442 asks the user to indicate if the food poster described below included in the system was followed and what the user ate from the list of foods recommended to be avoided. The second level 1444 asks the user to indicate if the user controlled serving size according to the recommendations of the system and what the user ate that went over the serving size limit. The third level 1446 asks the user to indicate if the user followed the meal plan and to record what and how much the user ate that was not on the meal plan.

In the moves or exercise section 1450, the user is instructed to record what kind of exercise 1452, how long each exercise was performed 1454 and how many steps until the user is short of breath 1456. The type of exercise, the amount of exercise and the number of steps until being short of breath may be recorded each day on the weekly medical record 1400. On a Sunday, for example, this information can be recorded in blanks 1462, 1464, and 1466, respectively. By recording the type and amount of exercise performed on various days of the week, the user is able to track progress from week to week.

In the med section 1460 of the “What I Did Section” 1406, the user is instructed to record in the weekly medical record 1402 illustrated in FIG. 4I, any variation in taking the medications the user has recorded in the meds section 1204 of the record book 106 illustrated in FIG. 4E and described above. Variations can include taking a medication that is not listed in the meds section 1204, taking a medication at a different time or not at all, or any other deviation from the plan.

As illustrated in FIG. 4I, the user is prompted to record any occurrence of an annoying cough each day on the weekly medical record 1400, and, on a Sunday for example, in blank 1472. The user is also prompted to record the number of cigarettes he/she smokes each day (if any) on the weekly medical record 1400, and, on a Sunday for example, in blank 1474. By recording the number of cigarettes smoked each day, the user is able to track progress from week to week. The user is further prompted to record whether their shoes or belt feel tighter than usual 1376. This may be recorded each day on the weekly medical record 1400, and, on a Sunday for example, in blank 1476.

Referring to FIG. 4J, the record book 106 includes a description of how a user can test his or her feet 1490. The instructions refer to a filament guide 146 which may or may not be provided with the system 100 to contact different areas of the bottom of the feet to determine if the user is able to detect the contact.

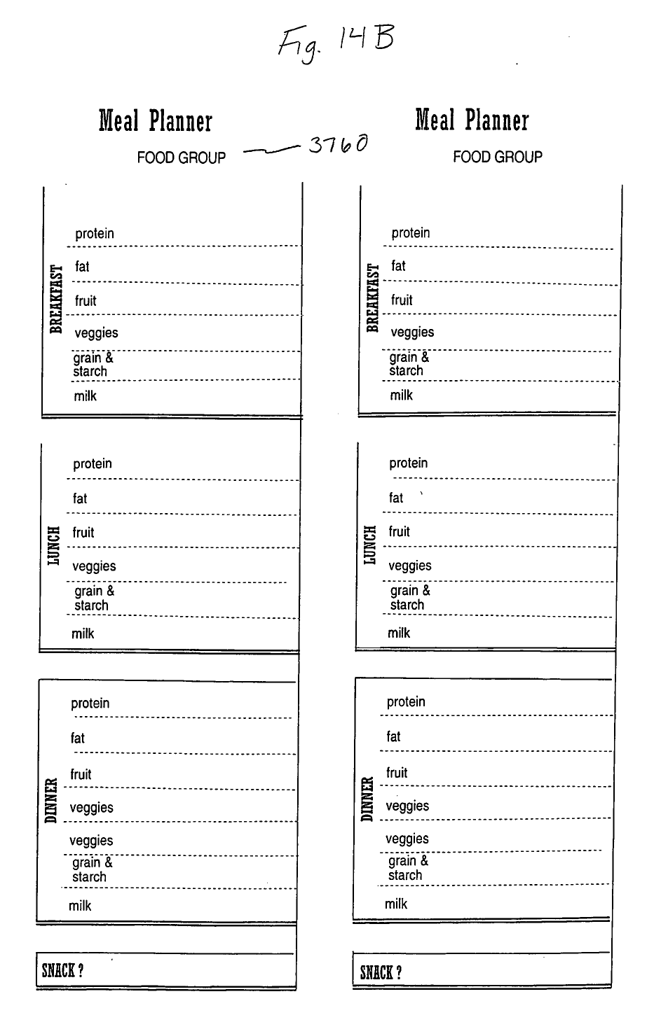

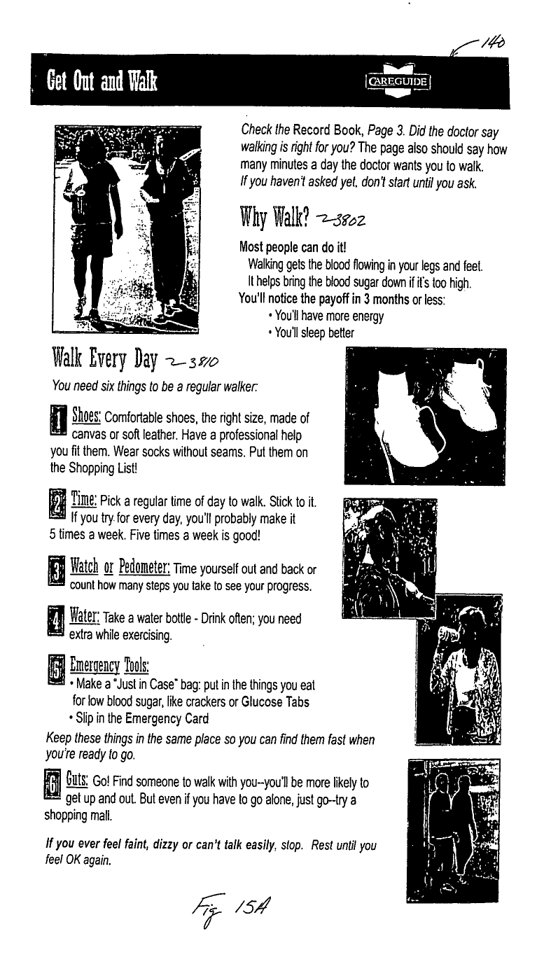

An example of a filament guide is illustrated in FIG. 14K. The filament guide may include a filament 1492 having a stiffness that enables the filament to stand erect at a length of one to two inches. The system 100 can include any suitable filament or probe with any suitable stiffness. The filament may be attached to a handle 1494 which provides a diagram illustrating areas on the feet to be tested with the filament 3872. The record book 106 also provides diagrams 1496 illustrating areas 1498 on the feet to be tested.

Referring back to FIG. 4J, the instructions instruct the user to touch the filament to an area of the foot indicated by a circle pushing hard enough for the filament to bend. The filament is held in place against the foot for approximately two seconds. If the user cannot feel the filament, the instructions instruct the user to place an “x” in the circle on the diagram 1498 in the record book 106. The instructions 1490 further instruct the user to repeat this method for each circle on both feet and to call the doctor if an “x” is indicated in any of the circles. In addition, the instructions include a list of months for the user to indicate when a foot test has been performed for that month.

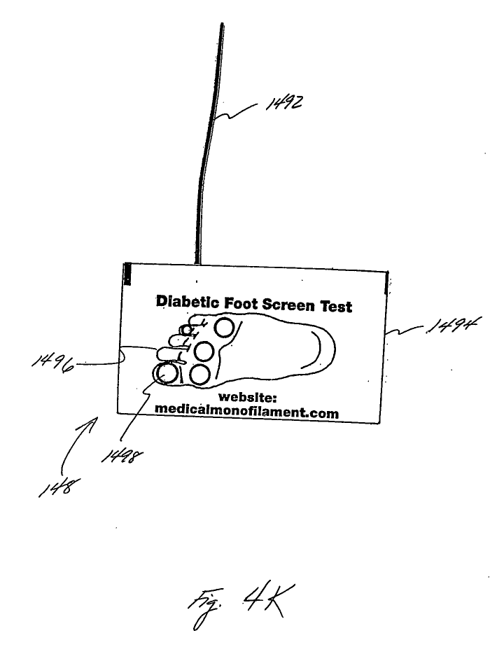

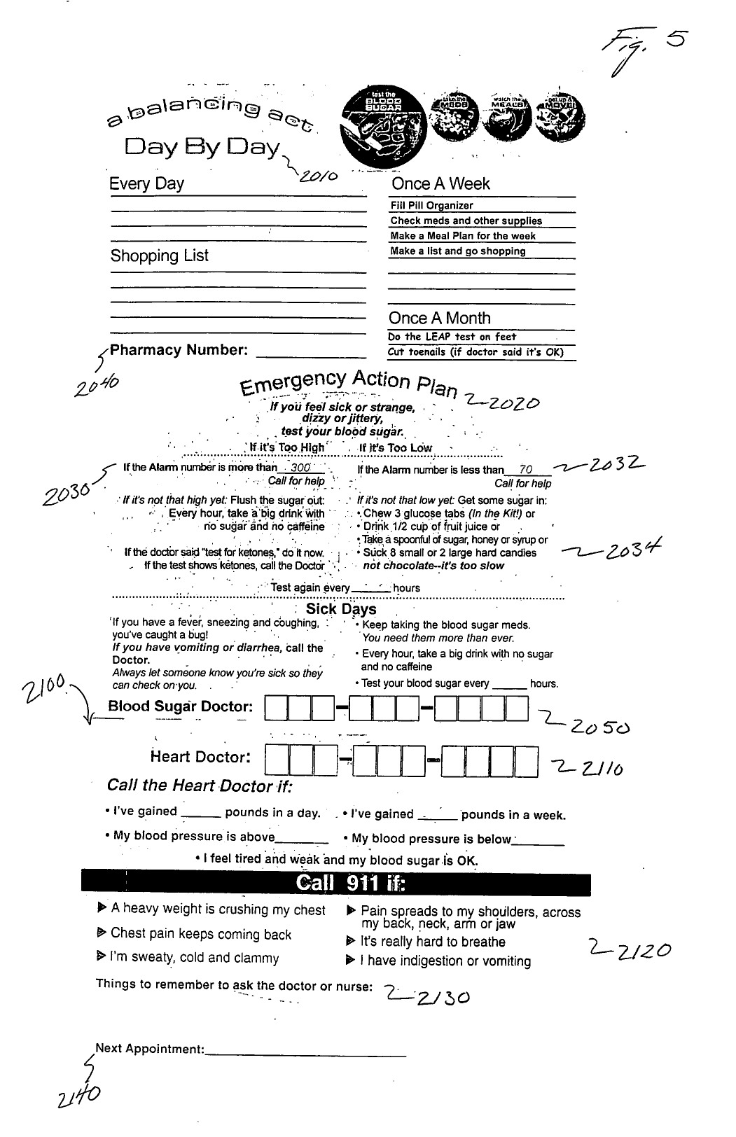

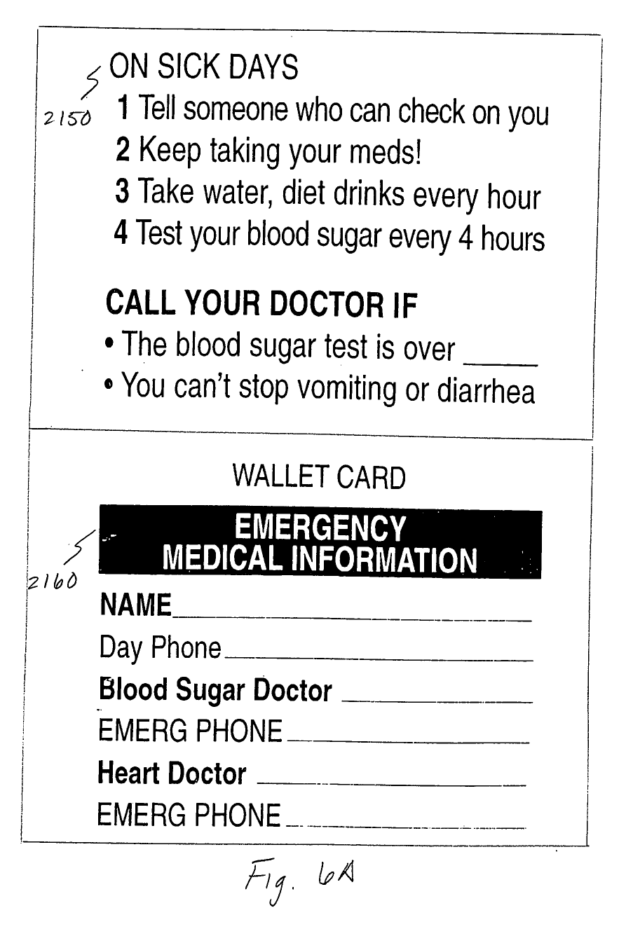

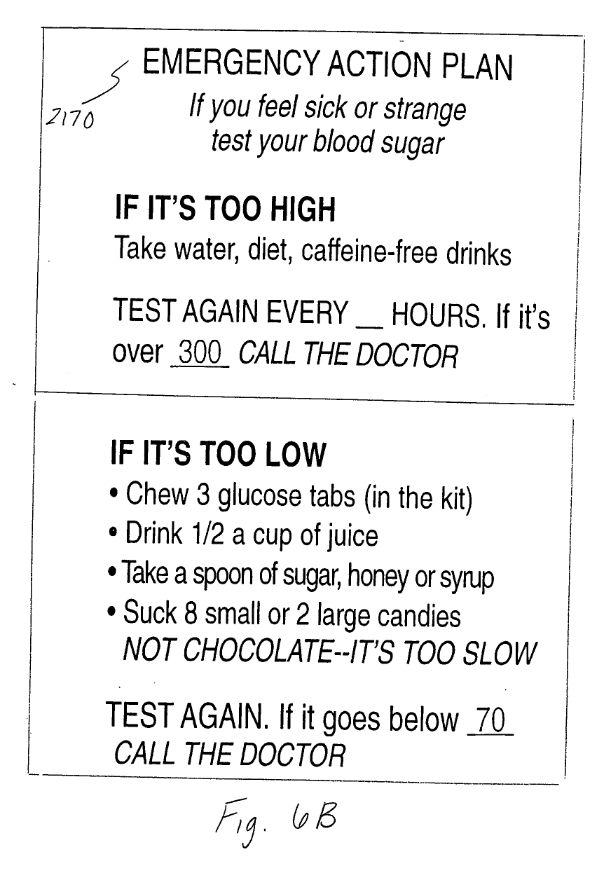

Returning to the second step 240 of the preparation guide 104 of the system 100, in addition to the record book 106, the second step 240 provides instructions 244 and 246 for a magnetic board 108 and a wallet card 110 illustrated in FIGS. 5 and 6A and 6B, respectively. The magnetic board 108 may be hung from a refrigerator as a convenient reference for information. In the illustrated embodiment, the information included on the magnet boards is divided into three sections. The first section 2010 includes a list of, and space to add, what needs to be done on a daily, weekly and monthly basis. The second section 2020 of the magnetic board 108 includes an emergency action plan tailored to respond to symptoms associated with abnormal blood sugar levels. The emergency action plan includes alarm numbers 1114 for high 2030 and low 2032 blood sugar levels provided by the health care provider and recorded in the assessment tests section 1100 of the record book 106 illustrated in FIG. 4B, along with instructions 2030 describing what to do if the symptoms associated with abnormal blood sugar levels occur. The emergency action plan also includes prescription refill telephone numbers 2040 along with an emergency telephone number 2050 for the blood sugar doctor.

A third section of the magnet board 108 includes a place to record the telephone number of the heart doctor 2110 and a list of events 2120 related to the health of the user. If one or more of the events 2120 occur, the user is instructed to call the heart doctor or call emergency medical services. The events may include a change for the worse, weight gain in excess of certain threshold limits in a day or a week, use of more pillows to sleep, worsening cough, swelling legs, difficulty breathing, blood pressure above or below certain threshold limits, feeling faint or dizzy with normal blood sugar, experiencing chest pains. The magnet board 108 also includes an area to note questions to ask the healthcare provider 2130 and a place to record information concerning the next appointment 2140.

Referring again to FIG. 2B, another tool described in the second step 240 of the preparation guide 104 of the system 100 includes a wallet card 110 illustrated in FIGS. 6A and 6B. The wallet card 110 may include a front and back side with specific instructions 2150 and emergency medical information 2160. The instructions may include what to do when the user is not feeling well, what to do 2170 if the user's blood sugar level exceeds certain specified limits including how to manage high or low blood sugar and when to call a doctor. The emergency medical information 2160 may include the identification and phone number of the user and names and phone numbers of doctors such as the heart doctor and blood sugar doctor.

As illustrated in FIG. 2C, step three 260 of the preparation guide 104 of the system 100 is to “Take the Tests.” In the illustrated embodiment, three tests are listed and described: the weight test 262, the blood pressure test 264 and the blood sugar test 266.

The weight test instructions 262 instructs the user to weigh himself/herself each morning before breakfast, and after using the toilet. The instructions 262 indicate that the user should place a scale on a hard floor (not carpeting) and to remove any clothing before weighing. A suitable set of scales 114 may be provided with the system 100. The weight is then recorded in a blank 1410 corresponding to the current day on the weekly record page 1402 as described above. The instructions 262 also tell the user to watch this recorded weight to make sure that the weight does not change (e.g., go up over time).

The blood pressure instructions 264 instruct the user to take his/her blood pressure every day and to record the blood pressure in the record book 106. A suitable blood pressure monitor 116 may be provided with the system 100. The blood pressure may be recorded each day in two blanks 1412a and 1412b on the weekly record page 1402 as described above and illustrated in FIG. 4I. The first blank 1412a is for recording blood pressure during the heart's push state, and the second blank 1412b is for recording blood pressure during the heart's rest state. An additional space 1416 is provided for the user to enter a pulse reading each day. A digital blood pressure monitor 116 is included in the system 100 for measuring blood pressure, and instructions 118 for using the blood pressure monitor 116 are described below.

The blood sugar instructions 266 instruct the user to obtain a blood sugar monitor which may or may not be included in the system 100. In addition, the user is instructed to obtain a lancet, a test strip, a clean towel, soap, a writing instrument such as a pen and the record book. The preparation guide 104 further instructs the user to wash and dry his or her hands with warm water and soap, and not alcohol, at a clean place near a sink. The user is then instructed to stick the side of a finger and to follow the monitor instructions to obtain a blood sugar measurement. The user is then instructed to record in the space provided 1413 and illustrated in FIG. 4I where the user obtained a sample and the blood sugar reading. The blood sugar is recorded in spatial relation 1414 to the high and low blood sugar limits provided by the health care provider in the weekly record 1402 in the record book 106 illustrated in FIG. 4B.

FIG. 2D illustrates the fourth step 280 of the preparation guide 104. The fourth step includes making a plan. As indicated in step one 210 of the system 100, and in the record book 106, the plan includes three parts: “watch the meals,” “make the moves,” and “take the meds.” The Personal Health Folder includes a section for each of these parts where the user may formulate a plan. According to the preparation guide 104, the plan will enable the user to achieve the five goals discussed in the short story described above. The five goals include: to lighten the load 282 by eating less salt and taking water pills, to shorten the trip 284 by watching what you eat and exercising more, to widen the road 286 by stopping smoking, relaxing and taking heart pills, to clear the strip 288 by eating more fiber and less fat and taking cholesterol pills, and to keep it steady 290 by doing regular amounts of everything on time.

Once the user has created a plan, step four 280 of the preparation guide 104 describes how the record book 106 enables the user to keep track of what the user does to follow the plan in the “What I did” section 1406 of the record book 106 and how the plan is working in the “How I tested” 1404 section of the record book 106 illustrated in FIG. 4I. The user is also instructed in the preparation guide 104 to take the record book 106 to every clinic visit to enable the doctor to review the information with the patient to determine if the plan is working and make revisions if necessary. The preparation guide 104 further reminds the user to obtain from the health care provider the updated test scores and any changes to limit numbers, diet, exercise or medication.

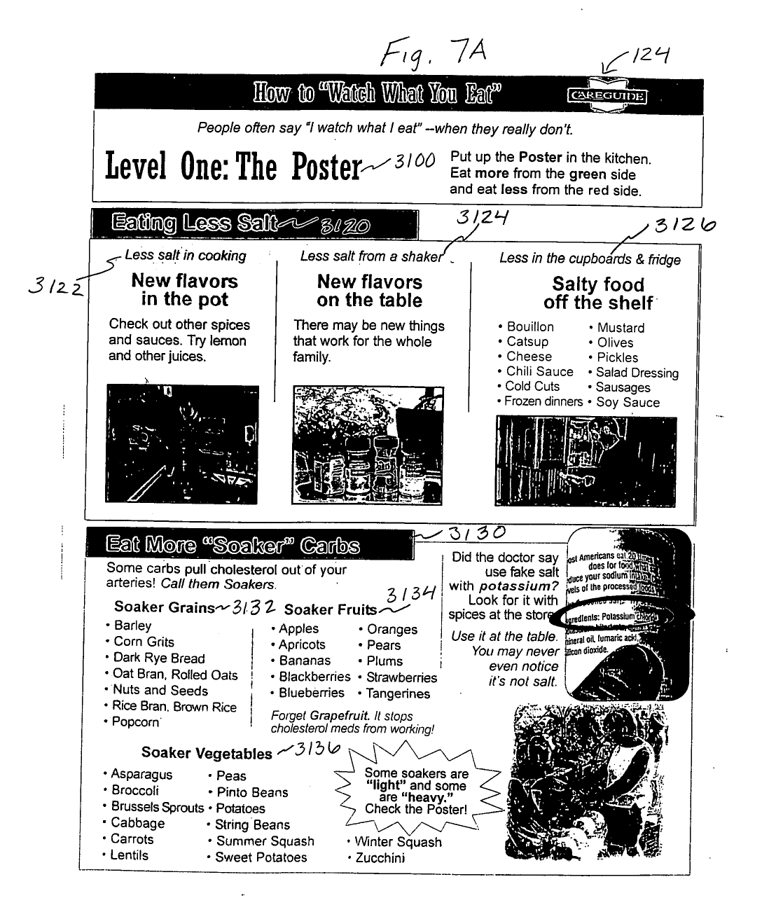

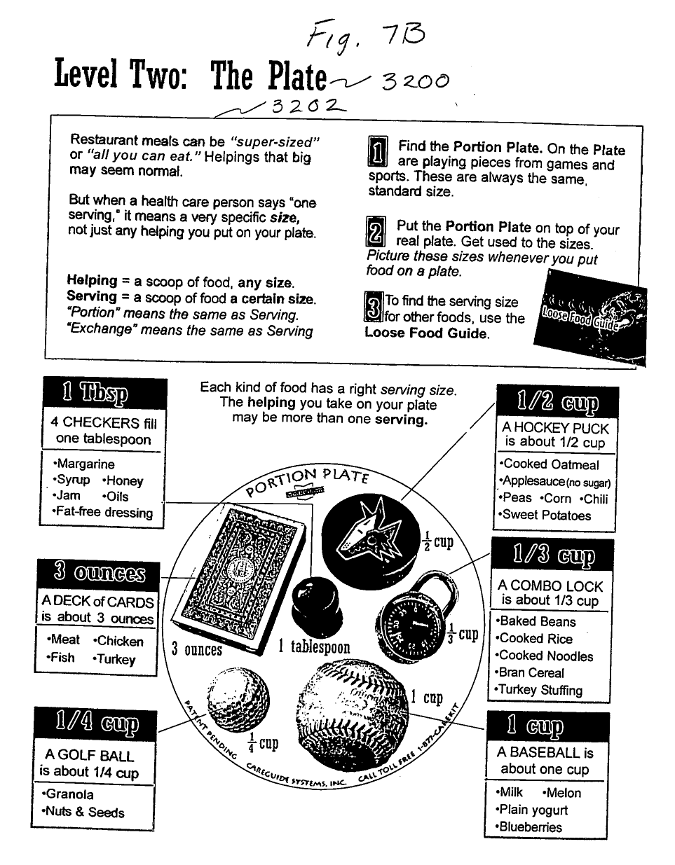

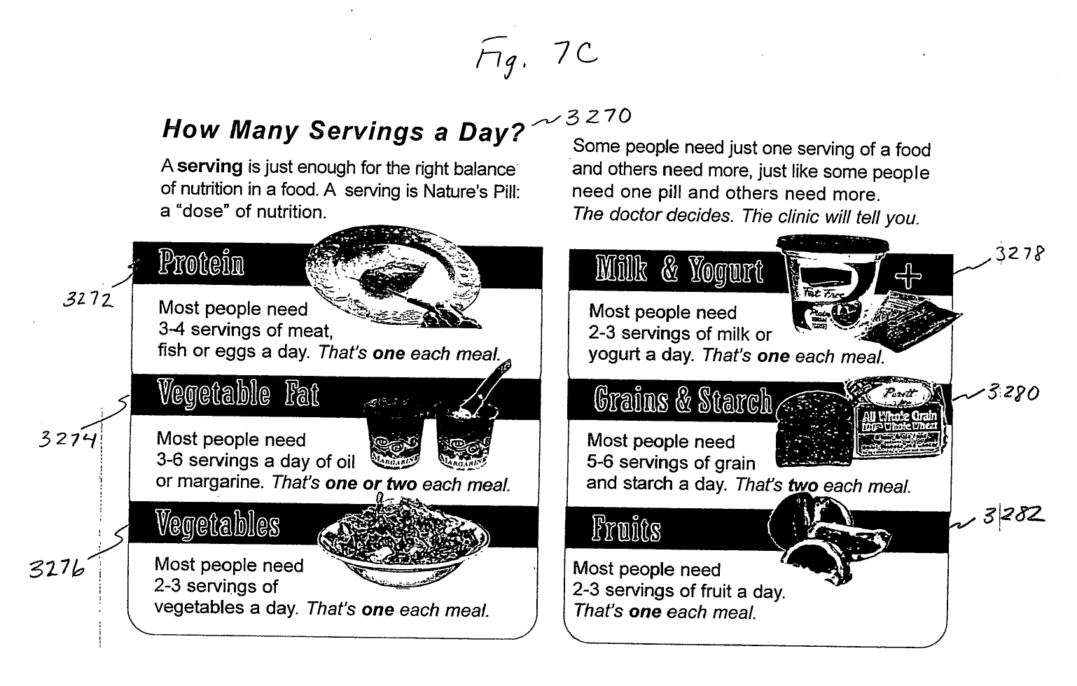

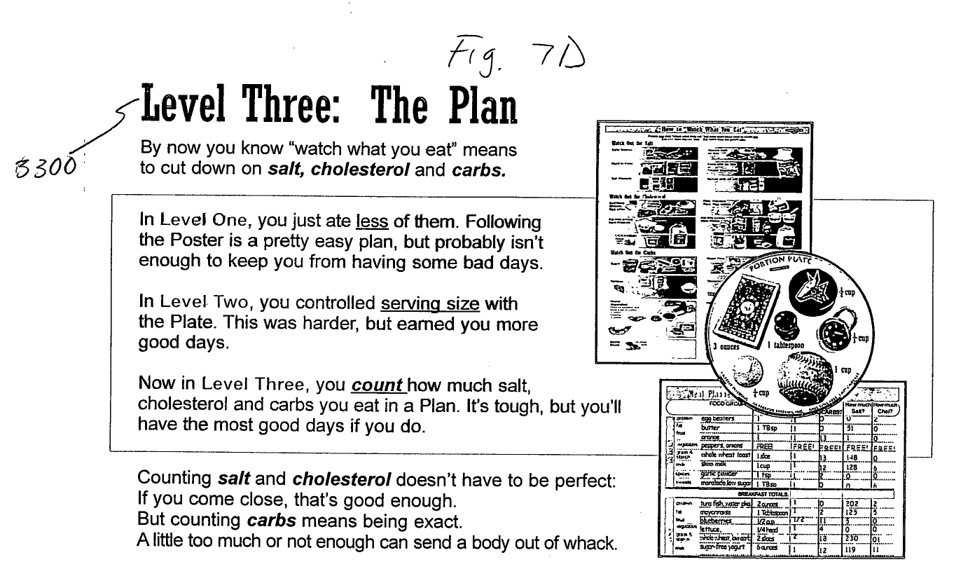

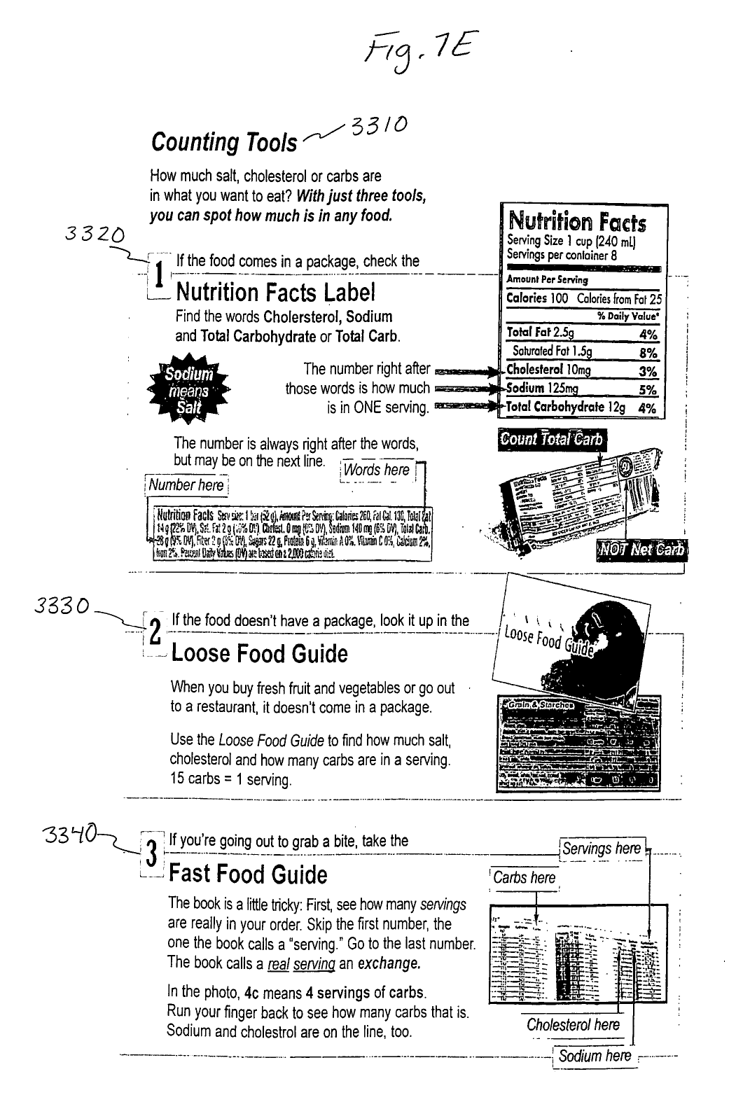

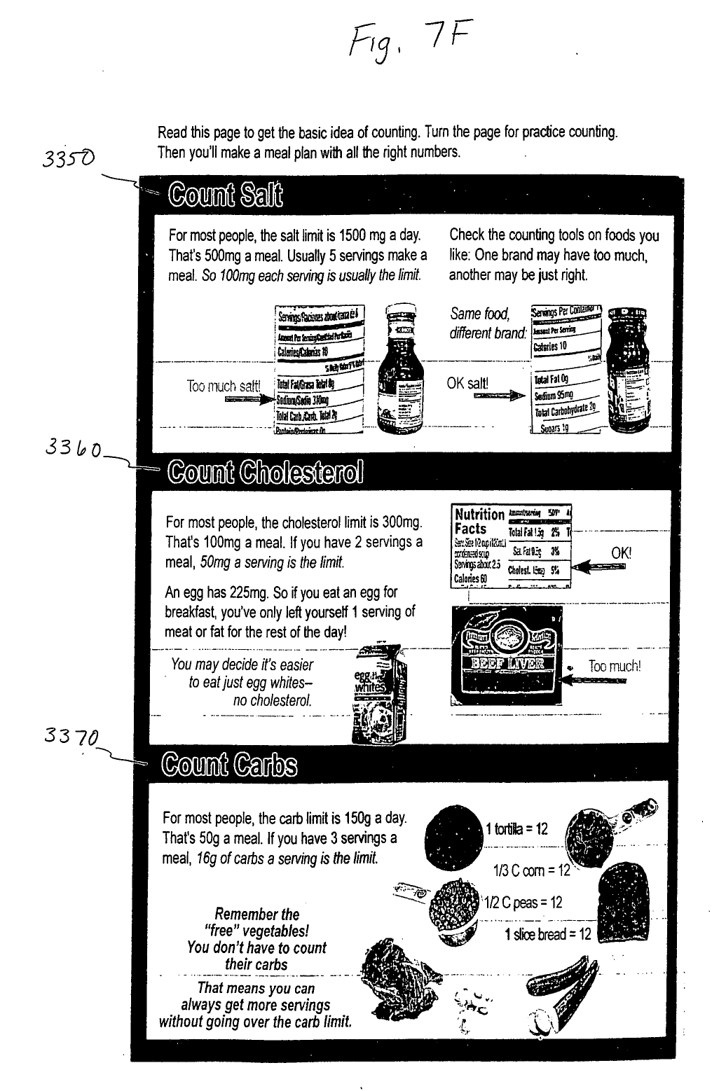

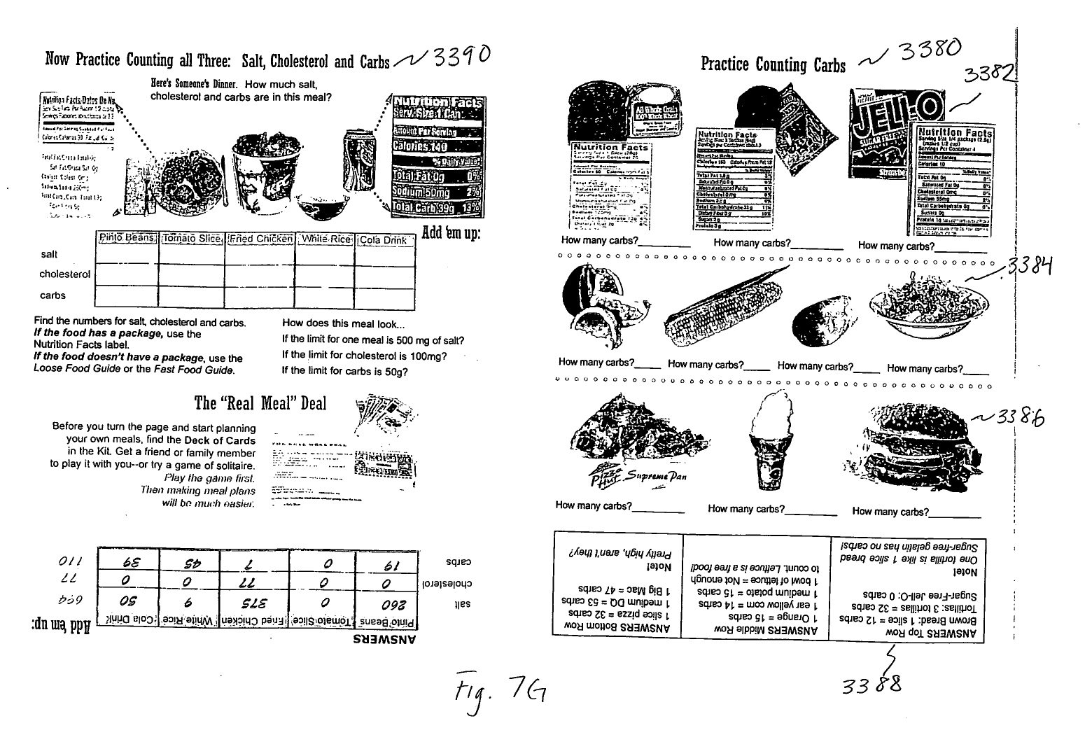

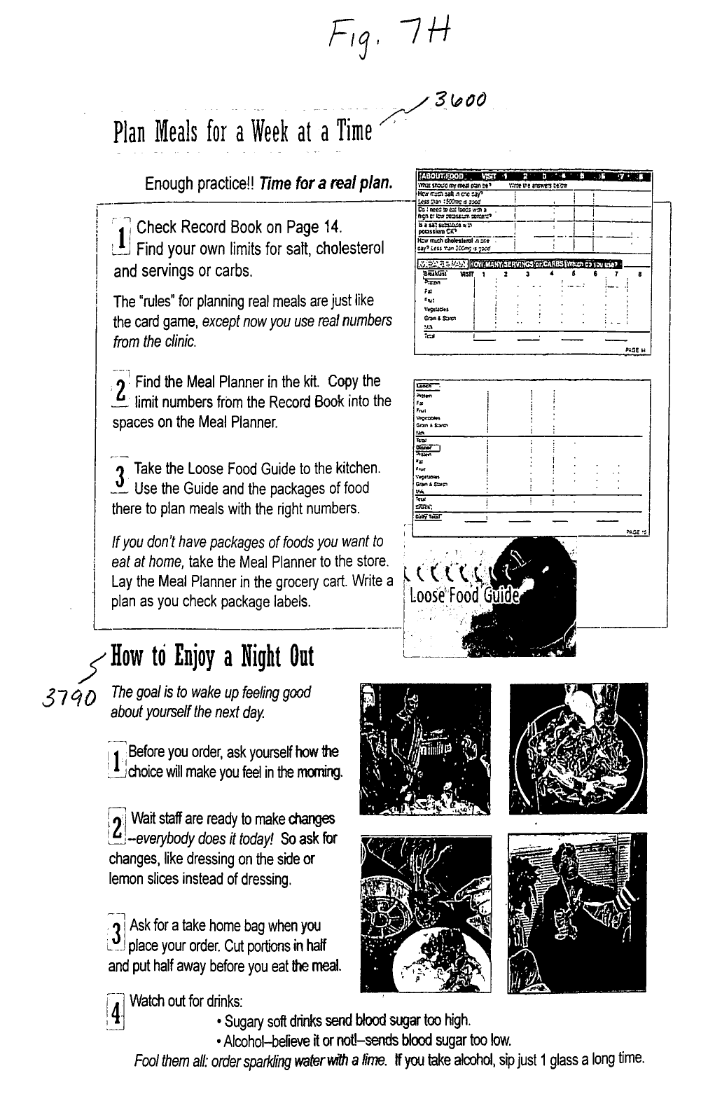

FIGS. 7A to 14B illustrate a meal program included in the system 100. The meal program includes a food guide 124, a food poster 126, a loose food guide 128, a fast food guide 130, a portion plate 132, a meal planner game 134, a meal planner 136, and a shopping list 138.

The meal program of the system 100 includes a food guide 124 illustrated at FIGS. 7A to 7G. The food guide 124 instructs the user of the system 100 how to watch what the user eats and, specifically, how to reduce salt, cholesterol and carbs in the diet. The food guide includes three levels: the poster 3100, the plate 3200, and the meal plan 3300. In the first level 3100 of the meal program, the user is taught how to eat less of salt, cholesterol and carbohydrates using a poster 126 that illustrates different foods categorized by their content. The second level 3200 of the meal program instructs or teaches the user how to control serving size using a portion plate 132 to illustrate the proper serving size for typical foods. The third level 3300 of the food guide teaches the user how to plan a menu for the day and to count how much salt, fat and carbohydrates are in foods eaten by the user.

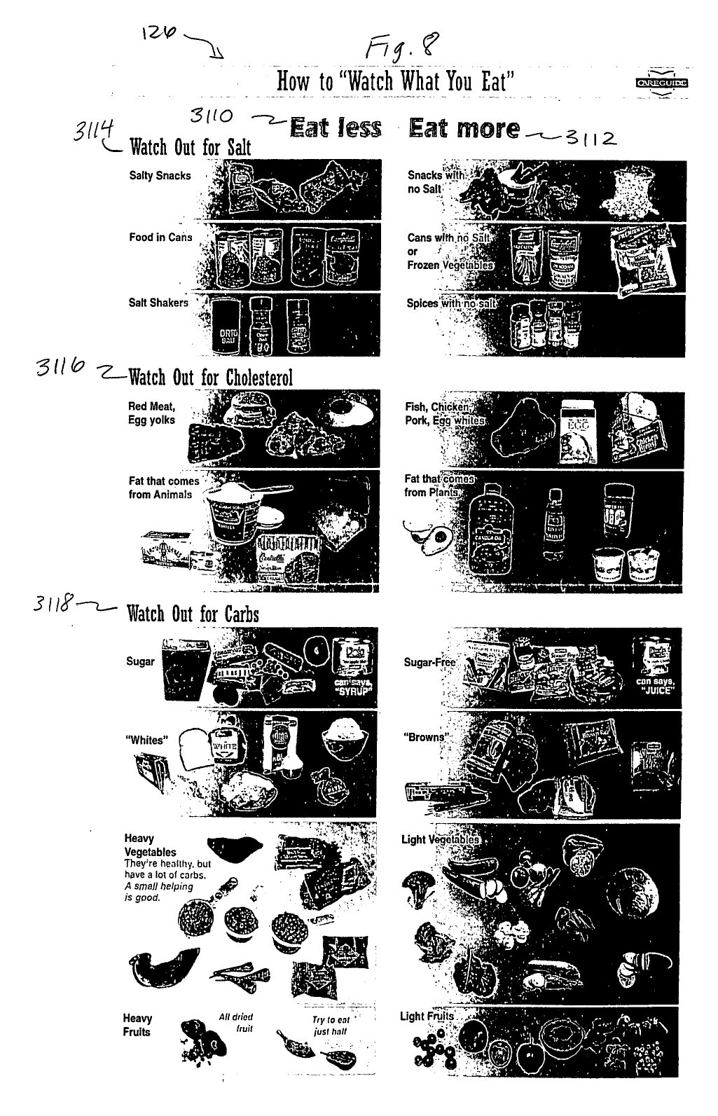

As described in level one 3100 of the food guide 124, a food poster 126 is included in the system 100 and is illustrated in FIG. 8. Referring to FIG. 8, the food poster 126 includes illustrations of food items. The food items are categorized by whether to eat more or less of the food. The food items are also categorized by what they contain or do not contain. The illustrated food poster is divided into a red section of foods 3110 and a green section of foods 3112 as an alternative to the foods in the red section 3110. The user is further instructed to eat more foods from the green side 3112 of the poster and eat less foods from the red side 3110 of the poster. The red or “eat less” 3110 section and the green or “eat more” 3112 section of the poster 126 are divided into three sections. The three sections include “watch out for salt” 3114, watch out for cholesterol” 3116 and “watch out for carbs” 3118.

In the “watch out for salt” section 3114, examples of food items containing salt in the red section 3110 and alternatives to those foods in the green section 3112 are illustrated. Examples of food products such as salty snacks, food in cans and salt shakers are illustrated as foods to eat less. Examples of food products such as snacks with no salt, canned food with no salt or frozen vegetables, and spices with no salt are illustrated as foods to eat more.

In the “watch out for cholesterol” section 3116, examples of food items containing cholesterol in the red section 3110 and alternatives to those foods in the green section 3112 are illustrated. Examples of red meat and egg yolks, and foods with fat from animals are illustrated as foods to eat less. Examples of fish chicken, pork and egg whites, and foods with fats that come from plants are illustrated as foods to eat more.

In the “watch out for carbs” section 3118, examples of food items containing carbs in the red section 3110 and alternatives to those foods in the green section 3112 are illustrated. Examples of sugar, white flour, heavy vegetables and heavy fruits are illustrated as foods to eat less. Examples of sugar-free foods, brown flour, light vegetables and light fruits are illustrated as foods to eat more. The user of the meal program is instructed by the food guide 124 to put up the poster 126 in the kitchen.

Referring again to the food guide 124 and FIG. 7A, the poster is explained or supplemented by instructions in the food guide to eat less salt 3120. The user is instructed to use less salt in cooking 3122, less salt from a shaker 3124, and less salt in the cupboards and refrigerator 3126. To use less salt in cooking, the food guide recommends using new flavors and other spices and sauces, such as lemon and other juices. In addition, the instructions recommend that the user remove salty food from the cupboards and refrigerator, such as bouillon, ketchup, cheese, chili sauce, cold cuts, frozen dinners, mustard, olives, pickles, salad dressing, sausages and soy sauce. The instructions also teach the user to get new flavors such as garlic, lemon and limes, basil, cilantro and onion into the home. As described above, the user may refer to the “About Food” section 1202 of the plan section 1200 of the record book 106 illustrated in FIG. 4D to determine how much salt a user is limited to in a single day and whether a potassium salt substitute is acceptable with a doctor's approval.

As illustrated in FIG. 7A, the food guide 124 discusses eating foods with potassium 3130. The food guide 124 provides a list of foods with high potassium 3132 for the user if the user has been advised by the doctor to eat foods with more potassium. Examples of food with high potassium include: apricots, avocado, banana, cantaloupe, honeydew, kiwi, lima beans, meat, poultry, fish, milk, oranges, juice, potatoes, prunes, spinach, tomatoes and winter squash. The instructions also provide a list of foods with low potassium 3134 for the user if the user has been advised by the doctor to eat foods with less potassium. Examples of food with low potassium include: apples, bell peppers, blueberries, cabbage, cranberries, cucumbers, fruit cocktail, grapes, green beans, iceberg lettuce, mushrooms, canned peaches, fresh pineapple and plums. In some cases, the user has been advised by the health provider to use fake salt with potassium. The food guide 124 illustrates where a user may look on a product to determine if the product contains potassium salt 3136.

As illustrated in FIG. 7B, the second level 3200 of the food guide 124 includes the plate 132. This section 3200 of the food guide 124 describes proper serving sizes for different types of food and a proper number of servings per day. The instructions 3202 discuss the difference between a “helping” and a “serving.” According to the instructions 3202, a helping includes a scoop of food of any size. In contrast, a serving or portion includes a scoop of food a certain size.

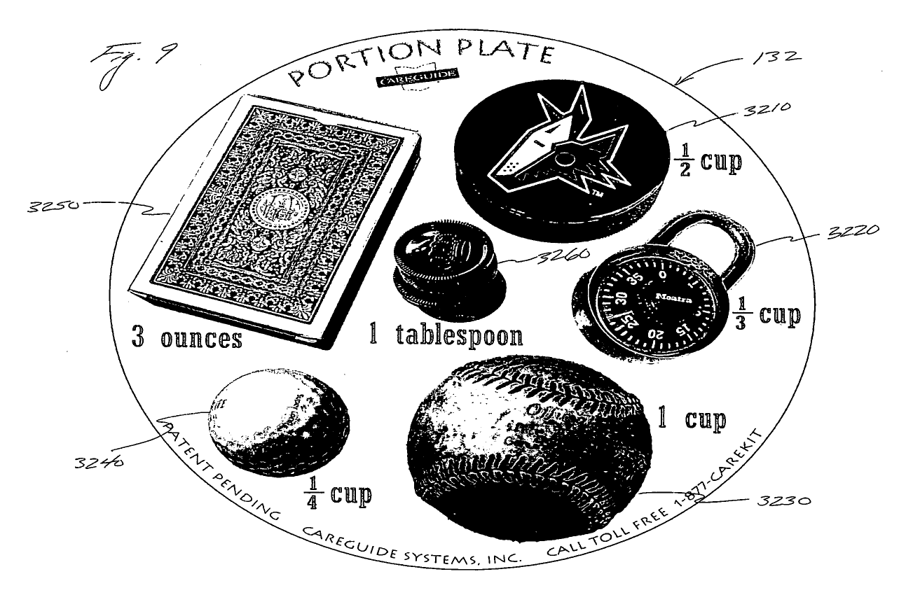

The food guide 124 employs a portion plate 132 illustrated at FIG. 9 to teach proper serving sizes for different types of food. Referring to FIG. 9, on the portion plate 132 are pictured playing pieces from games and sports providing the user a reference to different amounts included in a serving size. For example, a hockey puck 3210 represents about ½ cup; a combo lock 3220 represents about ⅓ cup; a baseball 3230 represents about 1 cup; a golf ball 3240 represents about a ¼ cup; a deck of cards 3250 represents about 3 ounces; and four stacked checkers 3260 represent about 1 tablespoon. The portion plate 132 also indicates typical foods that should be consumed by the user in the amount specified on the portion plate 132. For example, the instructions indicate that a proper serving size for cooked oatmeal, applesauce, peas, corn, chili or sweet potatoes is ½ cup, or the size of a hockey puck 3210 illustrated on the portion plate 132. The proper serving size for baked beans, cooked rice, cooked noodles, bran cereal or turkey stuffing is ⅓ a cup, or the size of the combo lock 3220 illustrated on the portion plate 132. The proper serving size for milk, melon, plain yogurt or blueberries can be 1 cup, or the size of the baseball 3230 illustrated on the portion plate 132. The proper serving size for granola or nuts and seeds can be ¼ cup, or the size of the golf ball illustrated on the portion plate 132. The proper serving size for meat, fish, chicken or turkey can be 3 ounces, or the size of a deck of cards 3250 illustrated on the portion plate 132. A proper serving size for margarine, syrup, honey, jam, oils or fat free dressing can be 1 tablespoon, or the size of four checkers 3260 illustrated on the portion plate 132.

Referring to FIG. 7C, after discussing serving size, the food guide discusses how many servings a user should have per day 3270. The instructions 3270 indicate that the number of servings may vary between different people. The instructions 3270 further illustrate different variations in serving sizes based on the type of food. The instructions list typical numbers of servings for protein 3272, vegetable fat 3274, vegetables 3276, milk and yogurt 3278, grains and starch 3280, and fruits 3282. For protein 3272, the instructions indicate that most people need three to four servings of meat, fish, or eggs a day, or one serving each meal. For vegetable fat 3274, the guide indicates that most people need three to six servings a day of oil or margarine, or one or two servings per meal. For vegetables 3276, the guide indicates that most people need two to three servings of vegetables a day, or one serving for each meal. For milk and yogurt 3278, the guide indicates that most people need two to three servings of milk or yogurt a day, or one serving for each meal. For grains and starch 3280, the guide indicates that most people need five to six serving of grains and starch a day, or two servings per meal. For fruits 3282, the guide indicates that most people need two to three servings of fruit a day, or one serving per meal.

As illustrated in FIG. 7D, the third level 3300 of the meal program described in the food guide 124 includes the plan. The food guide 124 teaches the user how to track the amount of salt, fat and carbohydrates the user consumes. In particular, the instructions provide a description of various counting tools 3310 the user of the program can use to count how much salt, cholesterol or carbohydrates are in certain foods.

The counting tool instructions 3310 illustrated in FIG. 7E include instructions for using the nutrition facts labels on a food package 3320, instructions 3330 for using the loose food guide 128 and the instructions for using the fast food guide 130. The nutrition facts label included on most food packages lists the amount of cholesterol, sodium or salt and carbohydrates or total carbohydrates.

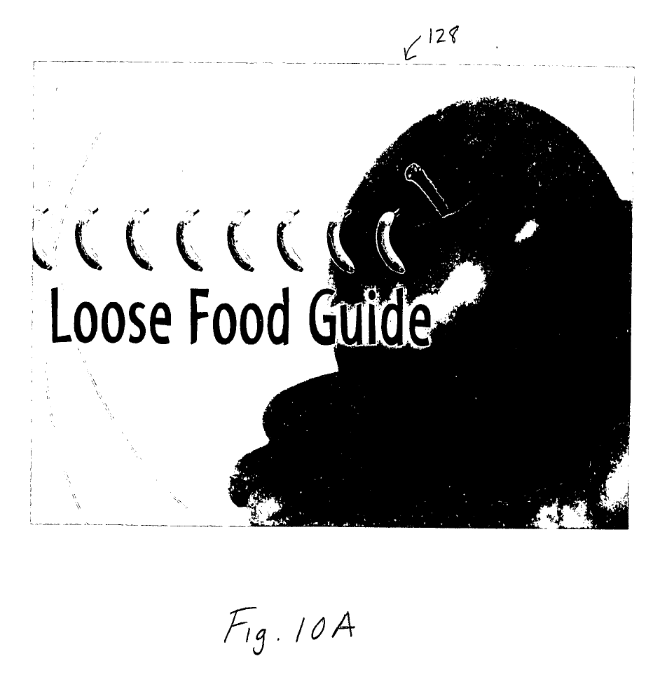

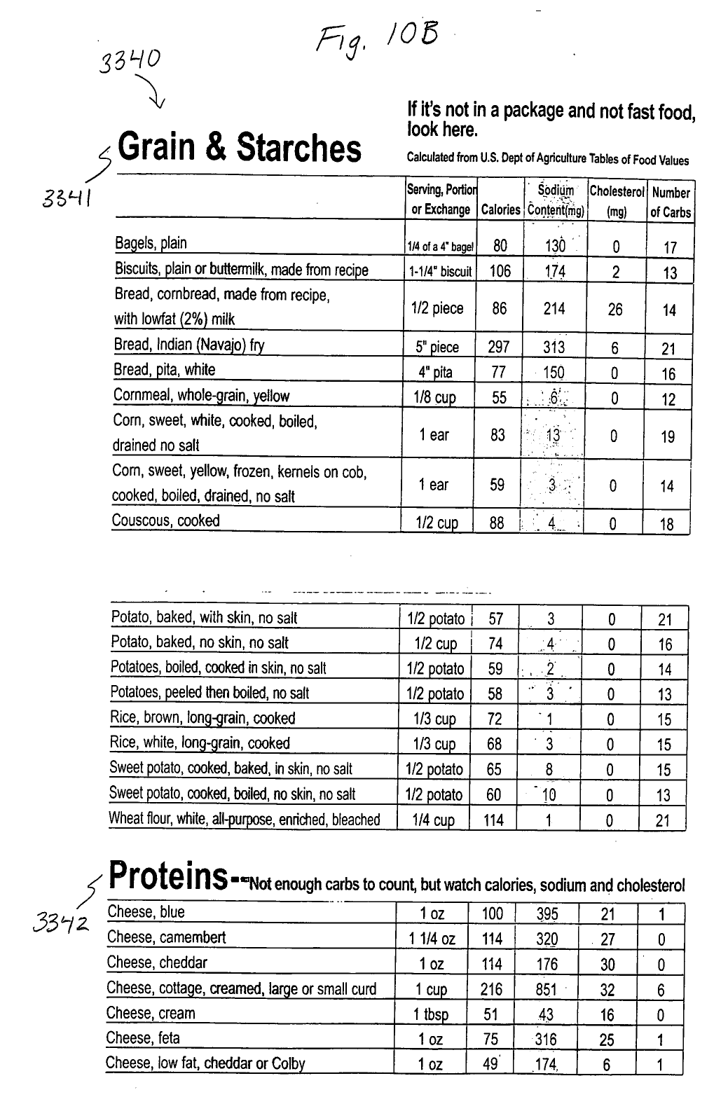

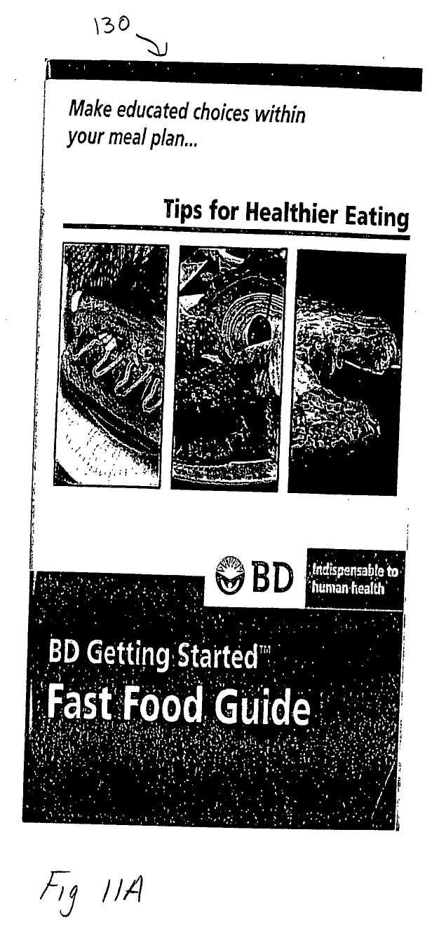

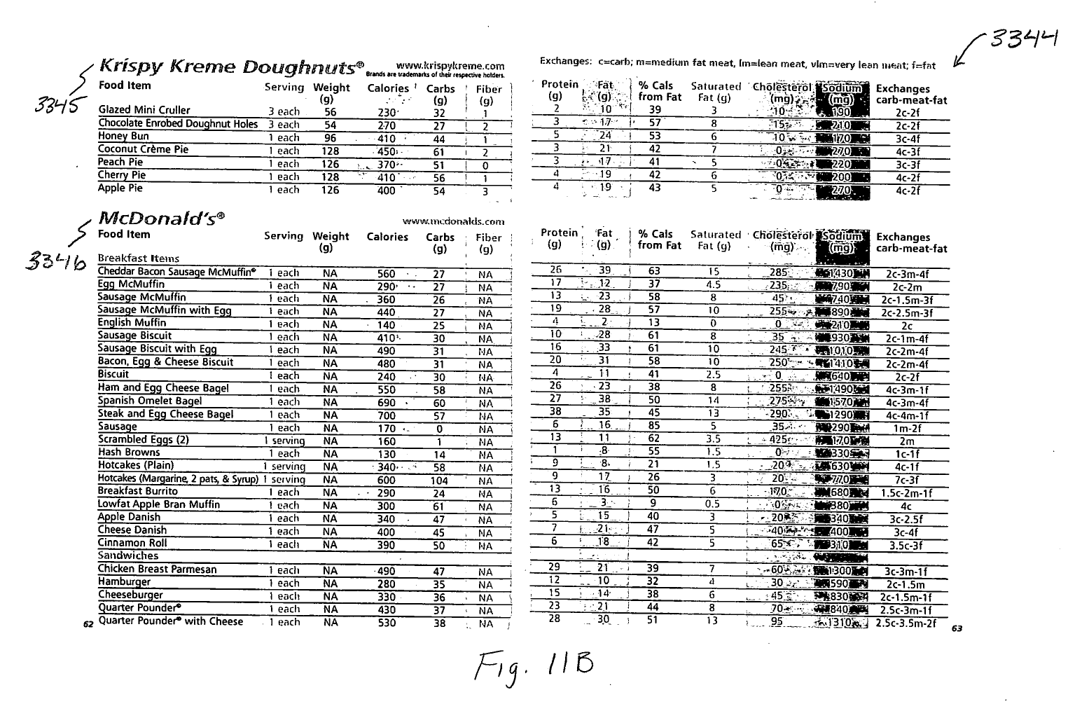

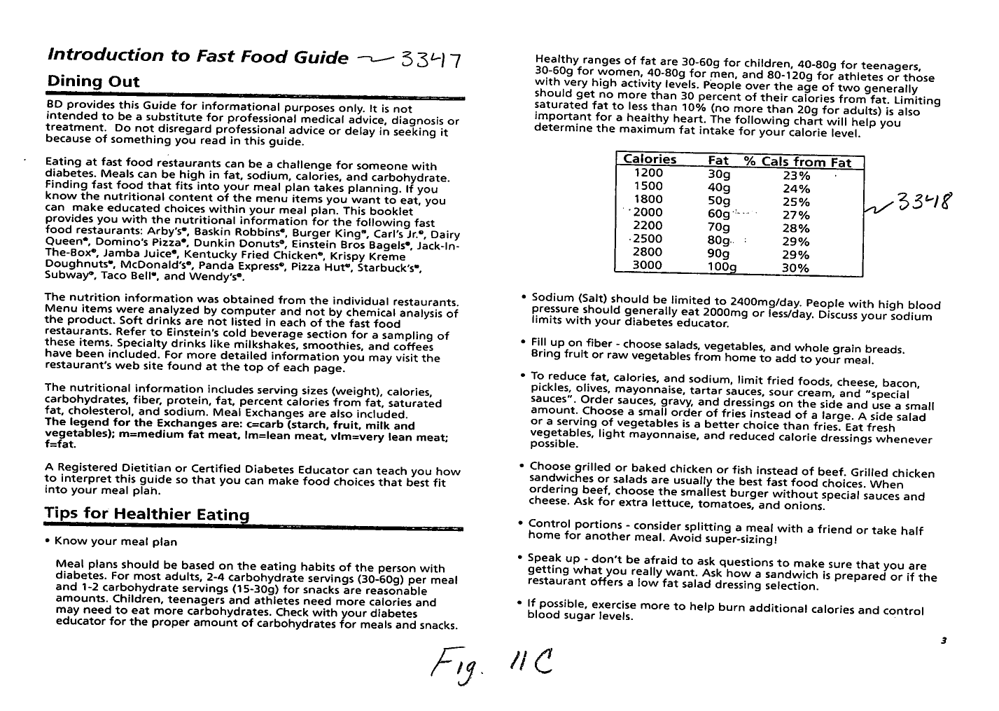

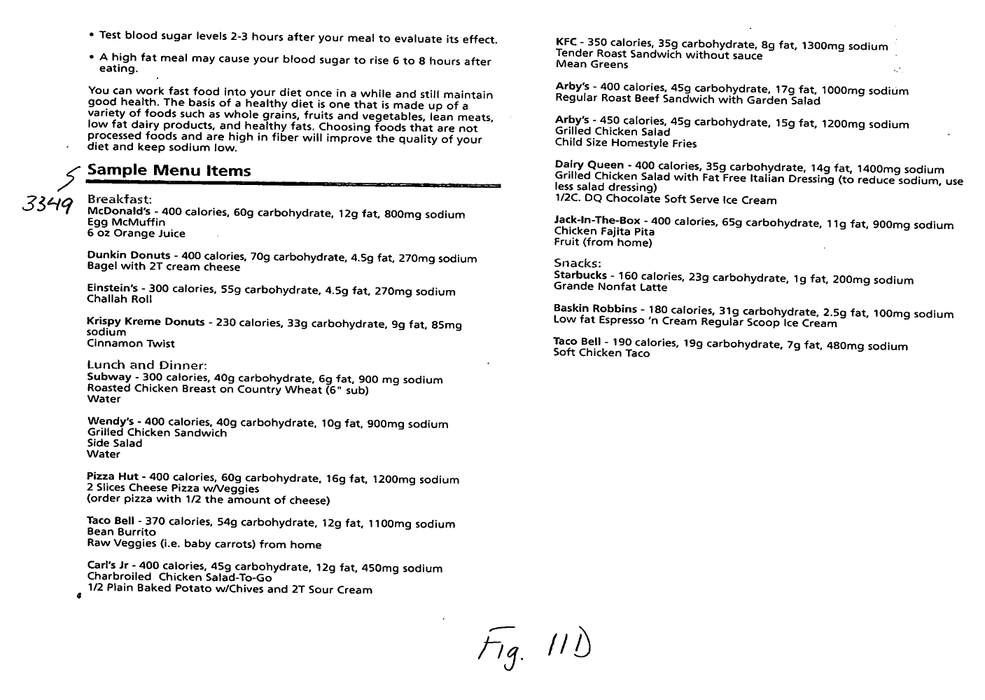

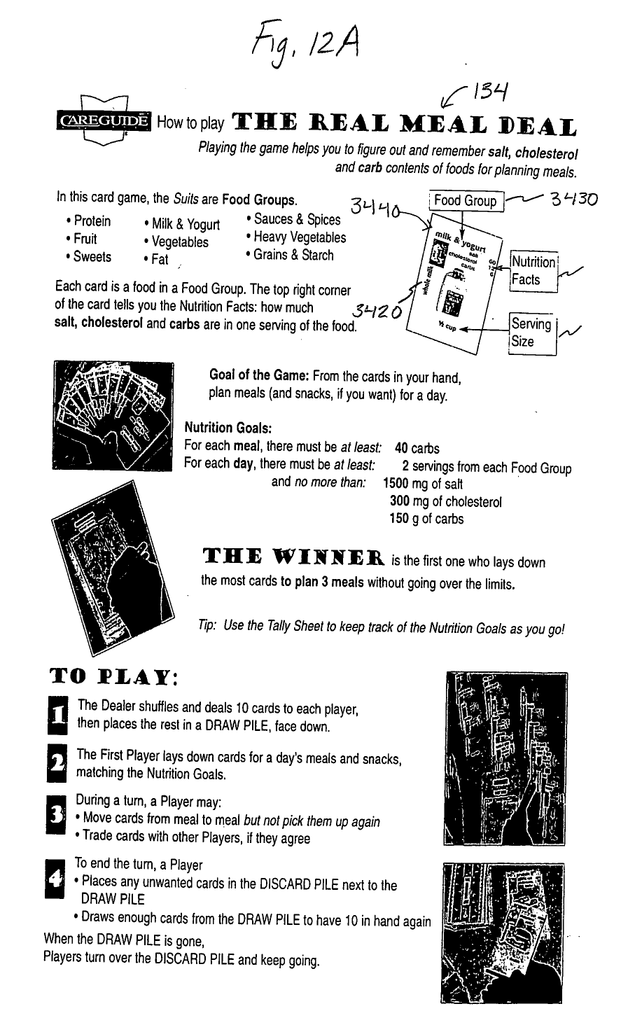

For those foods that may not have nutritional facts labeling, the system 100 includes a loose food guide 128 and a fast food guide 130 illustrated in FIGS. 10 and 11, respectively. As illustrated in FIGS. 10A and 10B, the loose food guide 128 enables the user to determine nutritional facts for foods typically purchased without packaging. The loose food guide 128 lists the calories, sodium, cholesterol, and carbohydrate content per serving, portion or exchange of grains and starches, proteins, fats and nuts, fruit, milk and yogurt, light and heavy vegetables, sweets and any other food that is not contained in a package. FIG. 10B illustrates an example of a table 3340 listing these amounts for grains and starches 3341, and proteins 3342.

Similarly, the fast food guide 130 included in the system 100 enables the user to determine the amount of calories, carbohydrates, fiber, protein, fat, percent of calories from fat, saturated fat, cholesterol, and sodium in a typical serving size, a real serving or exchange size of food items available at restaurants or fast food establishments. For example, FIG. 11B illustrates a table 3344 listing the nutritional amounts for food items at Krispy Kreme® 3345 and McDonalds® 3346. As illustrated in FIG. 11C, the fast food guide 130 also includes instructions on maintaining a healthy diet 3347 and a conversion table 3348 that enables the user to determine recommended fat intake for different calorie levels. In addition, the fast food guide 130 includes a listing 3349 of sample menu items from various fast food establishments that fall within certain nutritional parameters, as illustrated in FIG. 11D.

Referring back to the food guide 124, as illustrated in FIG. 7F, the food guide also includes examples of how to count salt 3350, cholesterol 3360 and carbohydrates 3370. The food guide instructions also include typical limits per day, per meal, and per serving for salt, cholesterol and carbohydrates. For example, according to the food guide the salt limit for most people is 1500 mg per day or 500 mg per meal. The counting salt instructions 3350 point out that, since there are approximately five servings per meal, each serving has a limit of 100 mg of salt. The instructions 3350 also point out that different brands of the same food product can often have different salt amounts. According to the food guide, the cholesterol limit for most people is 300 mg per day or 100 mg per meal. If a person has two servings of food with cholesterol in a meal, the limit for each serving is approximately 50 mg of cholesterol. According to the food guide, the carbohydrate limit for most people is approximately is 150 grams per day or 50 grams per meal. If the user has three servings of food that include carbohydrates per meal, the average number of carbohydrates per serving is 16. The instructions 3370 do not ask the user to count carbohydrates for “free” fruits and vegetables. The food guide also provides and illustrates examples of the number of carbohydrates associated with proper serving sizes of different foods.

As illustrated in FIG. 7G, the food guide 124 of the meal program provides a user an opportunity to practice counting carbohydrates in various food products. The food guide illustrates packaged 3382, loose food 3384 and fast food 3386 products and provides the number of carbohydrates in each of these products 3388. In addition, the meal program provides the user an opportunity to determine the amount of salt cholesterol and carbohydrates in an example meal 3390.

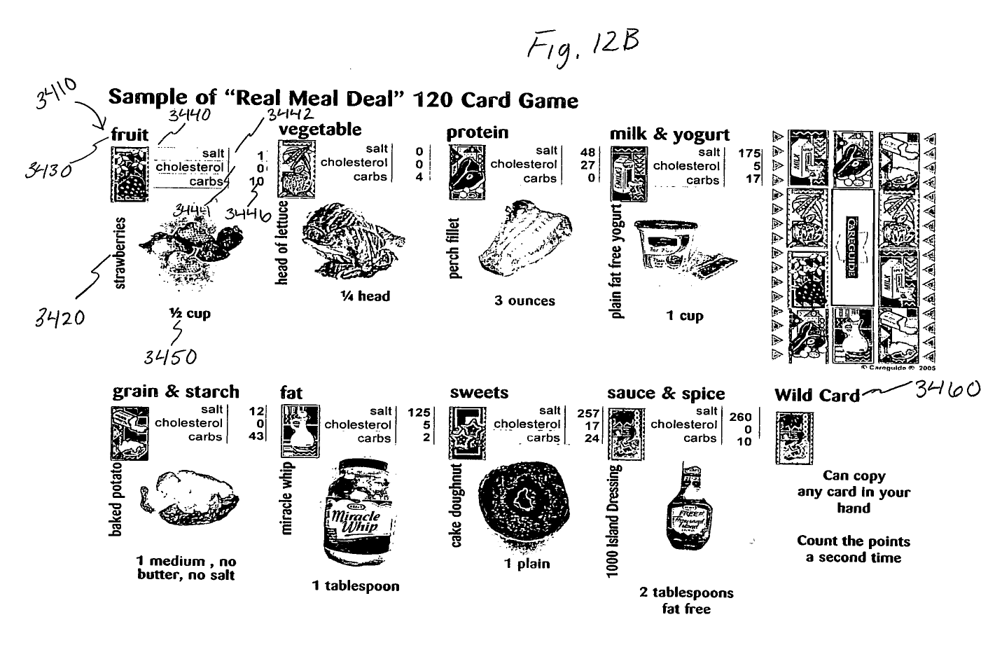

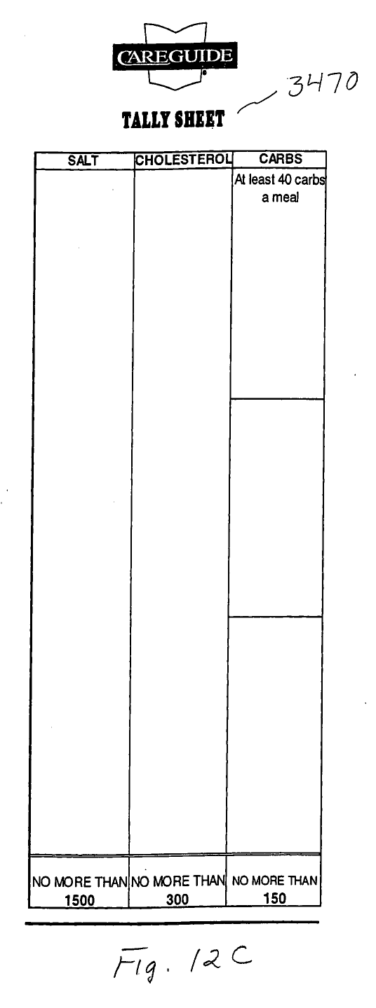

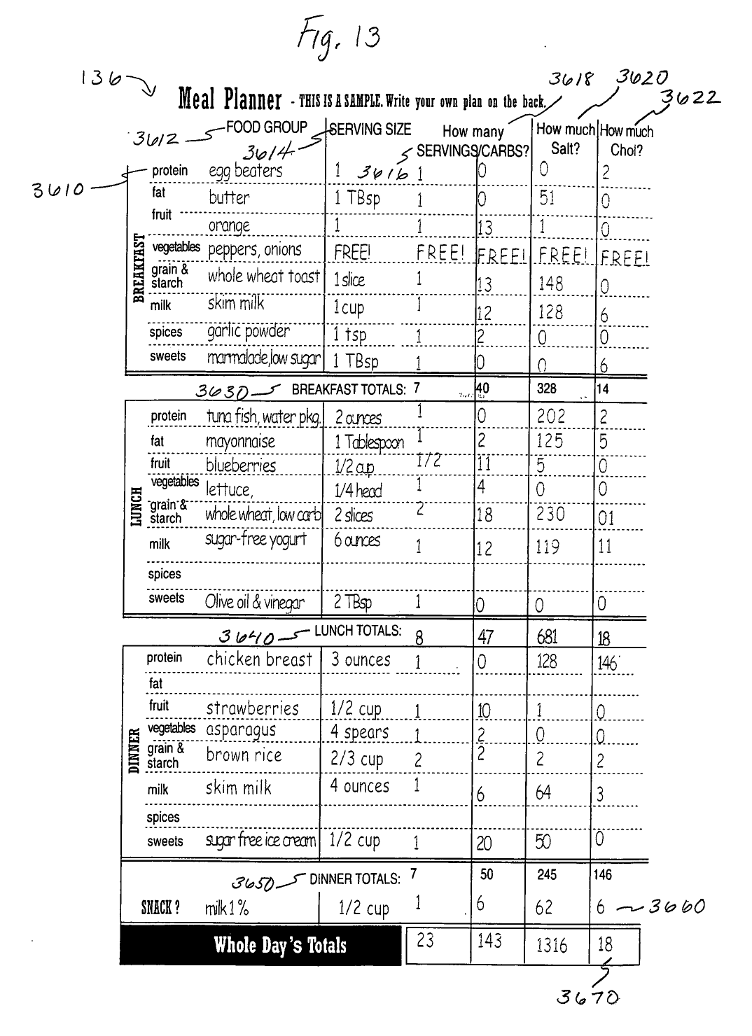

In one embodiment, the meal program includes a game, referred to in the food guide 124, to teach a user how to plan meals within the limits for the amount of salt, cholesterol and carbohydrates in each of those meals. In particular, FIGS. 12A to 12C illustrate a food selection or meal planner game 134 included in the meal program. The food selection game 134 is entitled “The Real Meal Deal” and enables a player to learn about salt, cholesterol and carbohydrate content in foods for planning meals. FIG. 12A illustrates instructions for the game. The game includes a plurality of cards. Examples of the cards are illustrated in FIG. 12B. Each card 3410 indicates a particular food or food product 3420 in a food group 3430 such as protein, fruit, sweets, milk & yogurt, vegetables fat, sauces and spices, heavy vegetables and grains and starch. In addition, the card includes nutrition facts 3440 of the food product 3420, and the serving size 3450 of the food product 3420. The nutrition facts are customized for the conditions being addressed by the system and include how much salt, cholesterol and carbohydrates are contained in one serving of the food product. For example, card 3430, illustrated in FIG. 12B indicates strawberries in the fruit food group. The nutrition facts 3440 include a salt content 3442 of 1, a cholesterol content 3444 of 0 and a carbohydrate content 3446 of 10 included in a serving size 3450 of one-half cup. FIG. 12B illustrates other examples of cards in the food selection game.

To begin the game, a dealer shuffles and deals to each player the same number of cards, such as ten cards. The remaining non-dealt cards are placed in a “Draw Pile” face down. Each player attempts to select from the ten cards indicating food products to be included in a meal or a snack. The goal of the game is to plan three meals without exceeding certain nutrition limits. In the illustrated embodiment, each meal must include 40 carbohydrates. The meals for each day may include at least two servings from at least one food group and contain no more than 1500 mg of salt, no more than 300 mg of cholesterol, and no more than 150 g of carbohydrates corresponding to the recommended daily limits described in the food guide and illustrated in FIG. 7F.

During play of the game, players take turns presenting cards for a day's meals and snacks which match the nutrition goals. The players are permitted to move cards from one meal to another meal but may not pick them up again. Players may also trade cards with other players if each agrees to the trade. As illustrated in FIG. 12B, a wild card 3460 may also be included in the game. The wild card 3460 indicates that the player may “copy any card in your hand” and “count the points a second time.” Therefore, the wild card enables a player to choose any card in his hand and count the numbers associated with salt, cholesterol and carbohydrate content a second time to meet the desired limits to be achieved in the game. Tally sheets 3470 illustrated in FIG. 12C may be provided to enable the players to keep track of the nutritional goals throughout the game.

A turn ends when the player places any unwanted cards in a “Discard Pile” and draws enough cards from the “Draw Pile” to replace the unwanted cards and to return the number of cards in the players hand to ten cards. When there are no more cards in the “Draw Pile,” the cards from the “Discard Pile” are then used. The first player who lays down the most cards to plan three meals without exceeding the nutritional limits is the winner. Once a winner has been declared, the game ends. Alternatively, play may continue to enable other players to attempt to plan three meals within the nutritional limits.

Once the user has had an opportunity to use the counting tools 3310 described in the food guide 124 and practiced planning meal with foods having less than the recommended limits for salt, cholesterol and carbohydrates, the user may use the meal planner 136 included in the system 100 to prepare actual meal plans. Referring to FIG. 7H, the food guide provides instruction 3600 in preparing meal plans that conform to the limits of serving size, number of servings and the amount of salt, cholesterol and carbohydrates in each of those meals. The food guide refers the user to the record book to assist the user in determining the limits recommended by the health care provider that have been recorded in the record book.

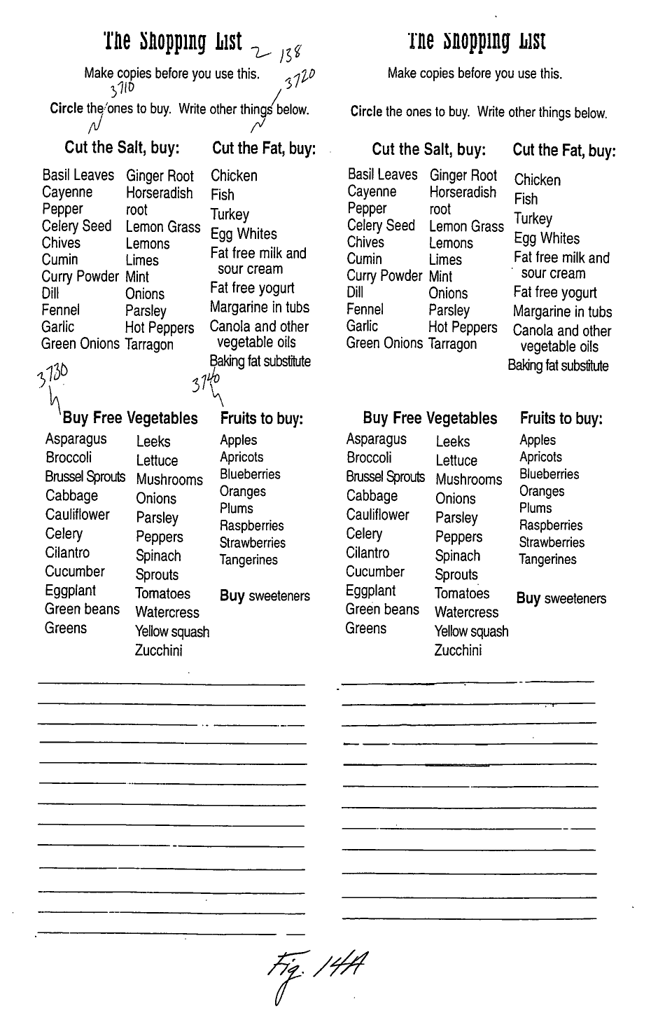

To assist the user in planning meals within these nutritional limits, the system includes a sample meal planner 136 illustrated in FIG. 13. The sample meal planner 136 lists for each meal the food groups, serving sizes for each of the food groups, how many servings in each of the food groups, how many carbohydrates in each of the food groups, how much salt in each of the food groups and how much cholesterol in each of the food groups. The food groups include: protein, fat, fruit, vegetables, grain and starch, milk, spices and sweets. In a meal planner without the example food information entered provided with the system 100, the meal planner 136 includes space to record numbers of servings 3616, total amounts of carbohydrates 3618 and amounts of salt 3620 and cholesterol 3622 from each of the foods and food groups represented in each meal. The meals include breakfast, lunch, dinner and snacks. Each of the breakfast totals 3630, lunch totals 3640, dinner totals 3650 and snack totals 3660 are added to determine the total for each of these parameters for the day 3670. Before the user completes the meal planner, the food guide instructs the user to copy the limit number for cholesterol and salt that have been entered into the plan section 1202 of the record book illustrated in FIG. 4D and described above. The limit numbers are recorded in the appropriate spaces on the meal planner. The user is instructed to plan meals within the proper limits based on the nutritional information on food product packaging and the information provided in the loose food guide 128.

The meal program also includes an example shopping list 138 and meal plans illustrated in FIGS. 14A and 14B. The shopping list 138 provides a list of alternative foods to those with salt 3710 and fat 3720 for easy reference when shopping in a grocery store. The shopping list also includes a listing of free vegetables 3730 and fruits 3740. The shopping list further includes an abbreviated meal planner 3760 with space to list foods in each food group for each meal including breakfast, lunch, dinner and snacks.

Referring back to FIG. 7H, the food guide 124 illustrates instructions on how to survive a night eating out 3790. For example, instead of regular salad dressing on a salad, the instructions 3790 suggest low fat salad dressing on the side or lemon juice squeezed from a lemon slice. In addition, the instructions suggest that the user cut the served portion in half and put half of the meal away before eating the meal. The instructions 3790 further advise the user that soft-drink consumption may cause blood sugar to increase above a normal level and that alcohol consumption may decrease blood sugar below a normal level.

The system 100 includes a move or exercise program. The exercise program includes a walking guide 140, a skin and foot care guide 142, skin lotion 144, mirror 146 and foot filament guide 148.

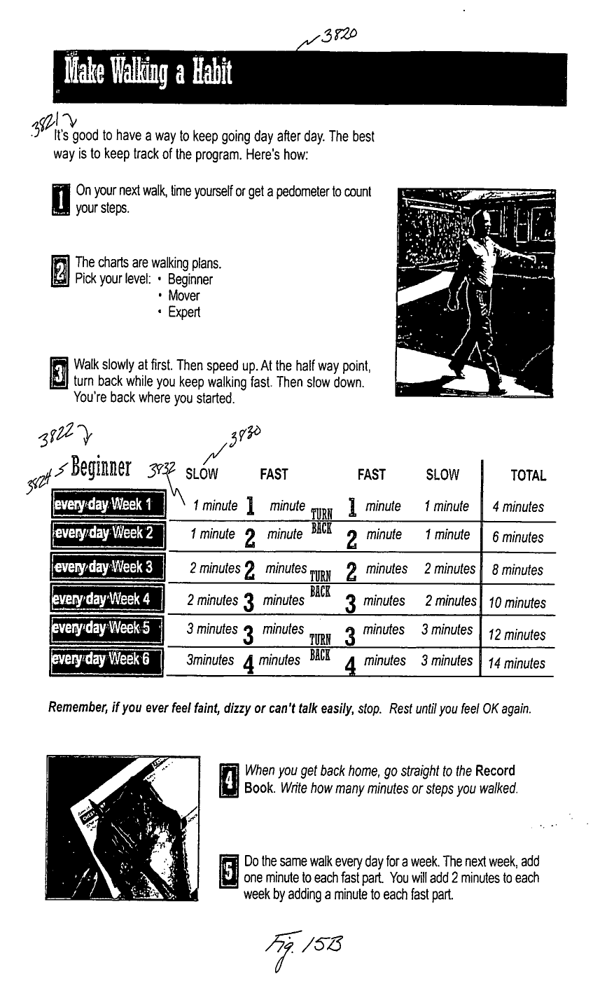

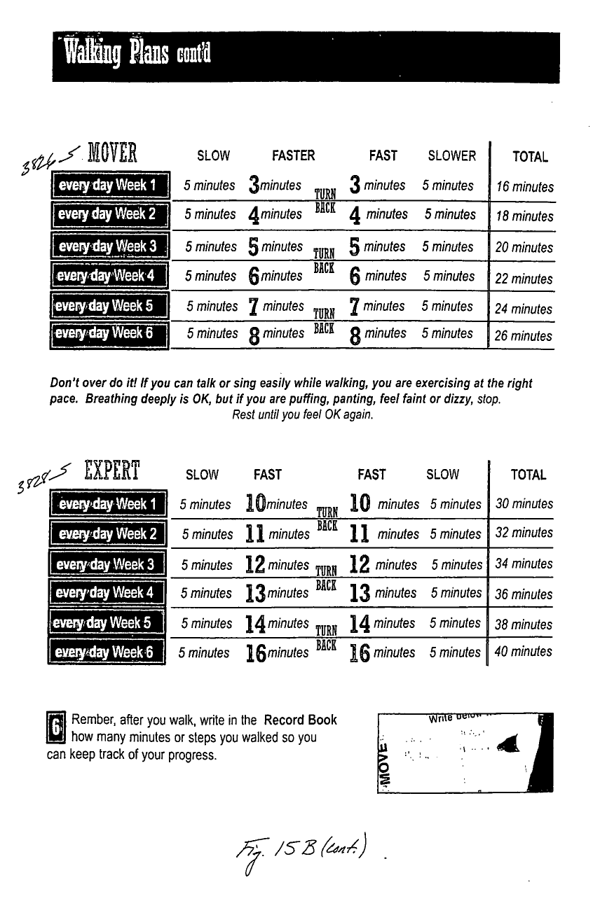

FIGS. 15A, 15B and 15C illustrate a secondary step-by-step walking guide 140. The secondary step-by-step walking guide 140 includes instructions on why people should walk 3802, items people need to have to be a regular walker 3810, how to make walking a habit 3820 and instructions for travel 3850.

FIG. 15A illustrates instructions on why people should walk 3802. The instructions 3802 inform the user that walking is good exercise that almost anyone can do. The instructions 3802 indicate that walking is good exercise because walking gets the blood flowing in the feet and legs. Walking also helps reduce high blood pressure. After several months of regular walking, the user can expect to have more energy and sleep better.

FIG. 15A also illustrates instructions on things people need to have to be a regular walker 3810. The instructions 3810 indicate that the user should have walking shoes, a regular walking time, a watch, water, and guts. The walking shoes should be the right size, comfortable, and made of canvas or soft leather. The user should walk at a regular walking time seven days per week and be content with sticking to the walking routine at least five times per week. The instructions also advise the user to use a watch and/or pedometer to measure time and distance or number of steps of the walk. The instructions further suggest taking a water bottle and to drink often. In addition the user is instructed to prepare a bag of emergency tools including the emergency wallet card 110 and what is necessary to treat any medical condition such as glucose tablets 56 or snacks to treat low blood sugar. The instructions 3810 also indicate that the user may want to find a friend to walk with. In this manner, the user may be more likely to stick to the walking commitment. Other suggestions included in the sample instructions 3810 include walking at a mall, not over-doing the walking routine, and walking at a pace where talking is still comfortable.

As illustrated in FIG. 15B, the “Make Walking a Habit 3820 section of the walking guide 140 presents instructions 3821 for using walking plans 3822 for different levels of walkers. In particular, the section includes three six-week programs for a “beginner” level 3824, a “mover” level 3826 and an “expert” level 3828. For each level and for each week, the plans 3822, include increments of relative speeds 3830, such as slow, slower, fast or faster, and the duration at each speed 3832. The plans 3822 also include at least two increments to walk in one direction and two increments to turn back and walk the opposite direction. For example, at the beginner level 3824 every day of the first week, the user is instructed to walk slow for one minute and fast for one minute. The user is then instructed to turn back and walk fast one minute and slow one minute for a total time of four minutes. At the expert level 3828 every day of sixth week, the user is instructed to walk slow for five minutes and fast for sixteen minutes. The user is then instructed to turn back and walk fast for sixteen minutes and slow for five minutes for a total time of forty minutes. The instructions 3821 throughout this section of the walking guide 140 advise the user what to do if warning signs such as feeling faint, dizzy occur. The user is also instructed to record in section 1203 of the plan section 1200 of the record book 104.

FIG. 15C illustrates advice on traveling 3840. The instructions 3820 include four steps. The first step is to obtain a hospital number to call in case of an emergency at the destination. The second step is to take double the amount of supplies that are anticipated. The third step is to pack the following in a small bag: (1) all needed supplies and medications; (2) the glucose tablets 56, included with the system 100 or snacks; (3) food with protein for a meal; (4) phone number for the user's health care provider; and (5) an emergency phone number at the destination. The fourth step is to obtain and wear an alert bracelet. The instructions 3820 also provide information on carrying insulin and blood sugar testing equipment on an airplane, including syringes, insulin pen, lancets and an insulin pump.

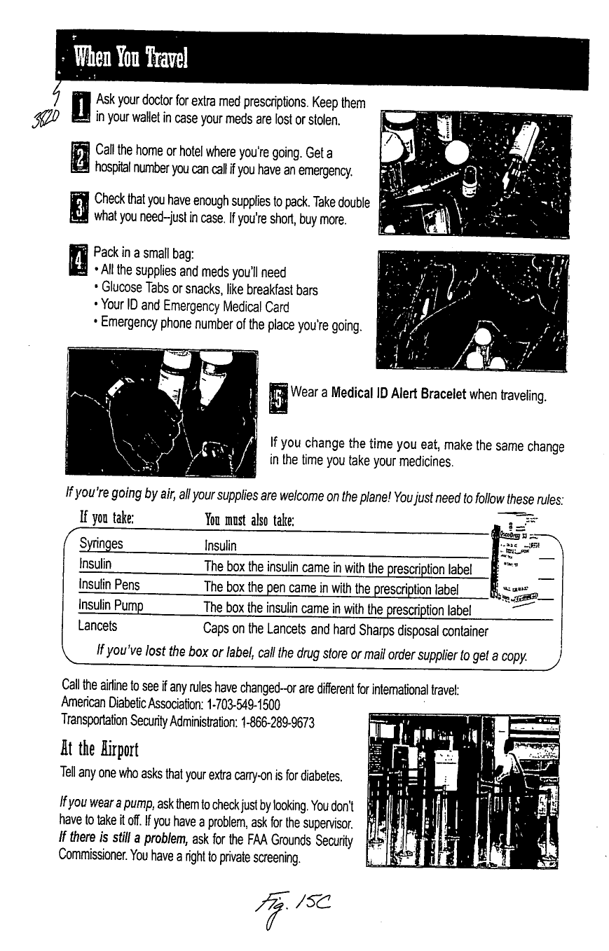

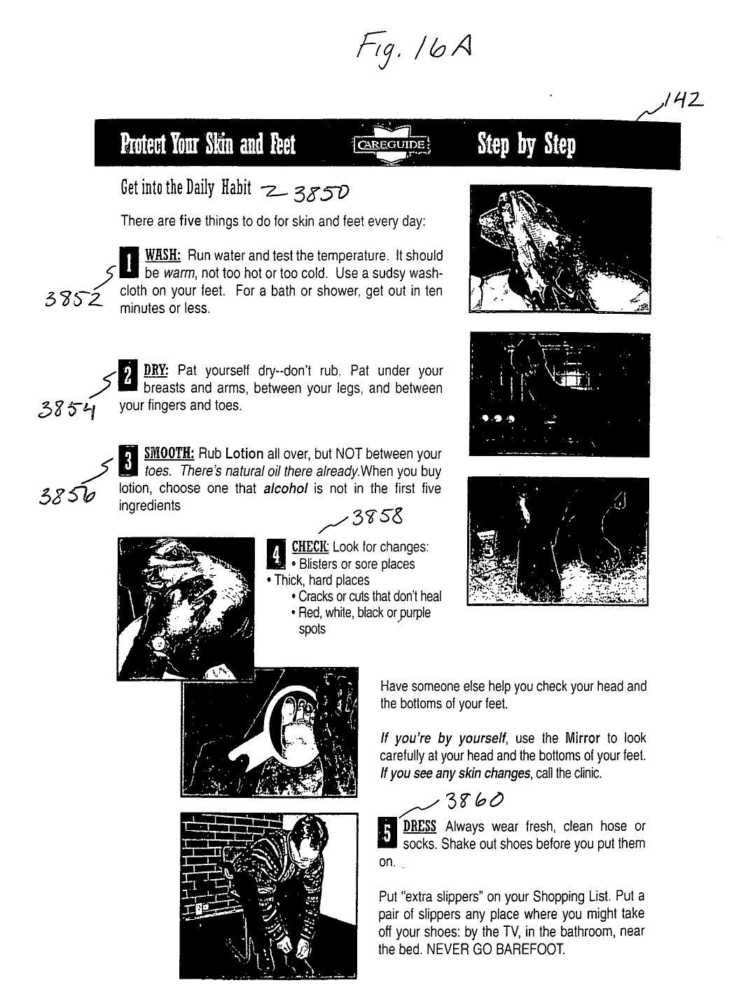

The exercise program of the system 100 also includes a step-by-step skin and foot care guide 142 illustrated in FIGS. 16A and 16B which provides instructions on how the user can protect skin and feet. The instructions are organized by how often different tasks should be performed. The instructions include daily instructions 3850, and instruction for other frequencies such as instructions for twice a week 3872, instructions for once a week 3874, instructions for once a month 3880, instructions for summer 3882 and instructions for winter 3884.

The “Protect Your Skin and Feet” instruction guide recommends five tasks that a user perform for skin and feet each day illustrated in FIG. 16A with explanatory photographs. The five tasks include: wash 3852, dry 3854, smooth 3856, check 3858 and dress 3860. The wash instruction 3852 instructs the user to run water and test the temperature. The water should be warm and not too hot or too cold. The instructions further provide that a user use a sudsy wash cloth on the feet and to limit the time in a bath or shower to ten minutes or less. The dry instructions 3854 instruct the user to pat dry the skin including hard to reach areas and not to rub the skin. The smooth instructions 3856 instruct the user to rub an alcohol-free skin lotion 144 provided in the system 100 over the entire body but not between the toes. In the illustrated embodiment, the skin lotion 144 is provided. The check instruction 3858 instructs the user to look for changes including blisters or sore places, thick hard places, cracks or cuts that don't heal, and red, white, black or purple spots. The instructions 3858 also suggest that the user have someone else check areas that cannot be visualized by the user. In the illustrated embodiment, the system 100 includes a mirror 146 for the user to use to look carefully at areas, such as the head, bottoms of feet and any other areas of the body difficult to view without the mirror 146. The mirror 146 included with the system 100 can be any suitable mirror. The user is instructed to contact the health care provider if the user notices any skin changes. The dress instruction 3860 instructs the user to always wear fresh, clean hose or socks and to shake out shoes before putting them on. The instructions 3860 also recommend purchasing extra slippers and placing the slippers any place wear the user might take off his or her shoes such as by a TV, in the bathroom, near a bed, to avoid going barefoot.

Additional instructions 3870 included in the skin and feet guide include washing hair at least twice a week 3872. If the user's health care provider permits, as documented in the record book 106 illustrated at FIG. 4D, the instructions 3870 advise users to cut their toenails once a week 3874 after a bath or shower. The instructions 3870 also provide a description and an illustration of the type of clippers the user should use. The instructions 3870 recommend straight-edge toenail clippers and provide a detailed description on how to cut the toenails. The system 100 may or may not include a pair of clippers.

The instructions further recommend that the user perform a leap test 2880 once a month and to indicate in the record book when the leap test is performed in accordance with the description of FIG. 4J. The leap test includes sticking the bottom of the foot to determine if the stick is felt by the user and when the stick is felt by the user.

The summer instructions 3882 recommend that the user use sunscreen which may or may not be included in the system 100 with SPF 15 or higher, to wear long sleeves and a hat, and to use bug spray when the user goes outside. The winter instructions 3884 recommend that the user use a humidifier or put bowls of water near the heat registers to prevent dry skin from cracking. The instructions further advise the user to bundle up when the user goes outside.

The system 100 includes a medication program. The medication program includes a medication guide 150, an insulin administration guide 152, a pill organizer 154 and glucose tablets 156.

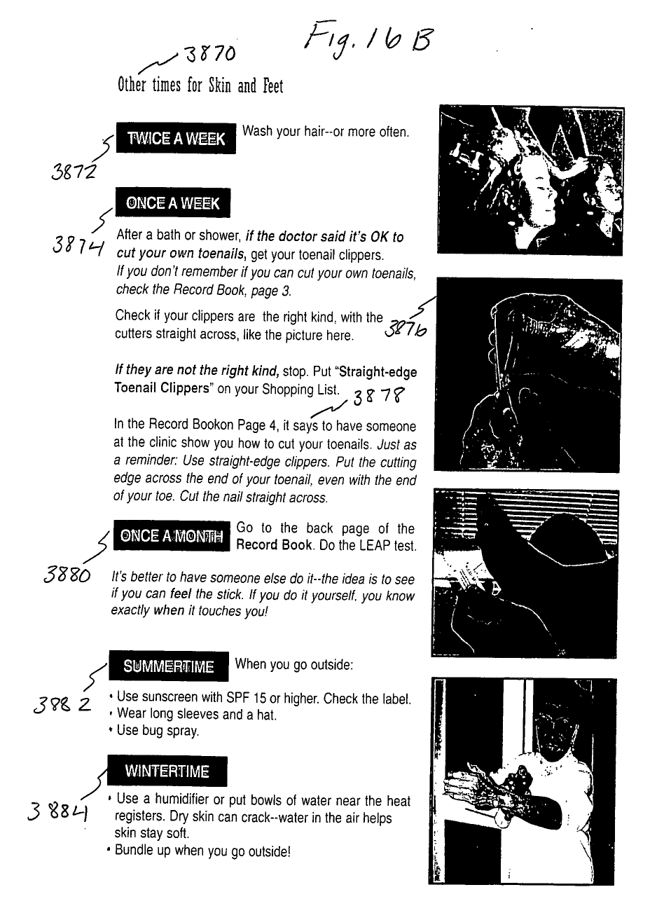

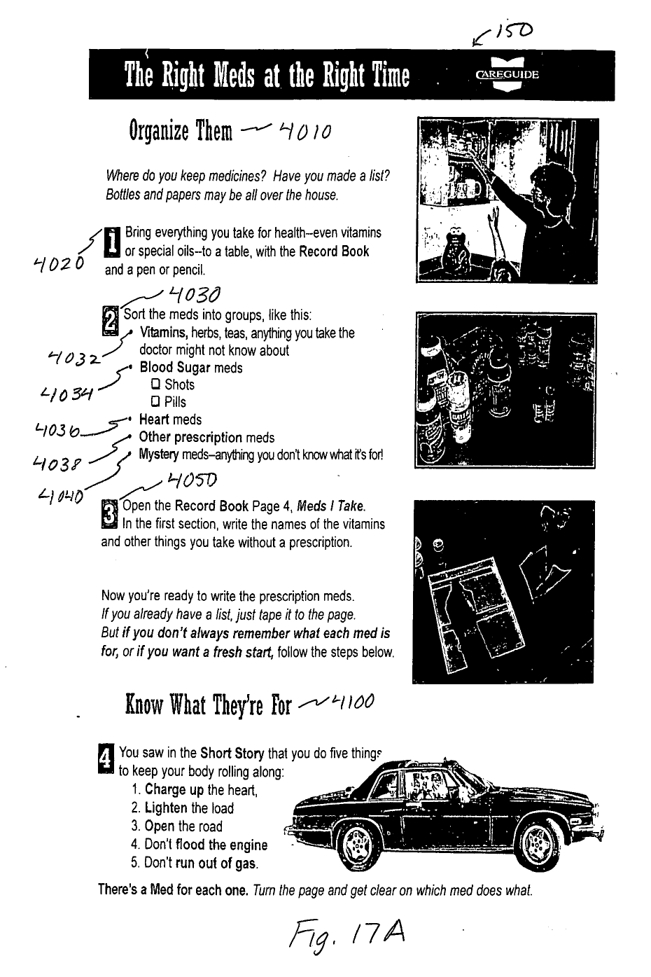

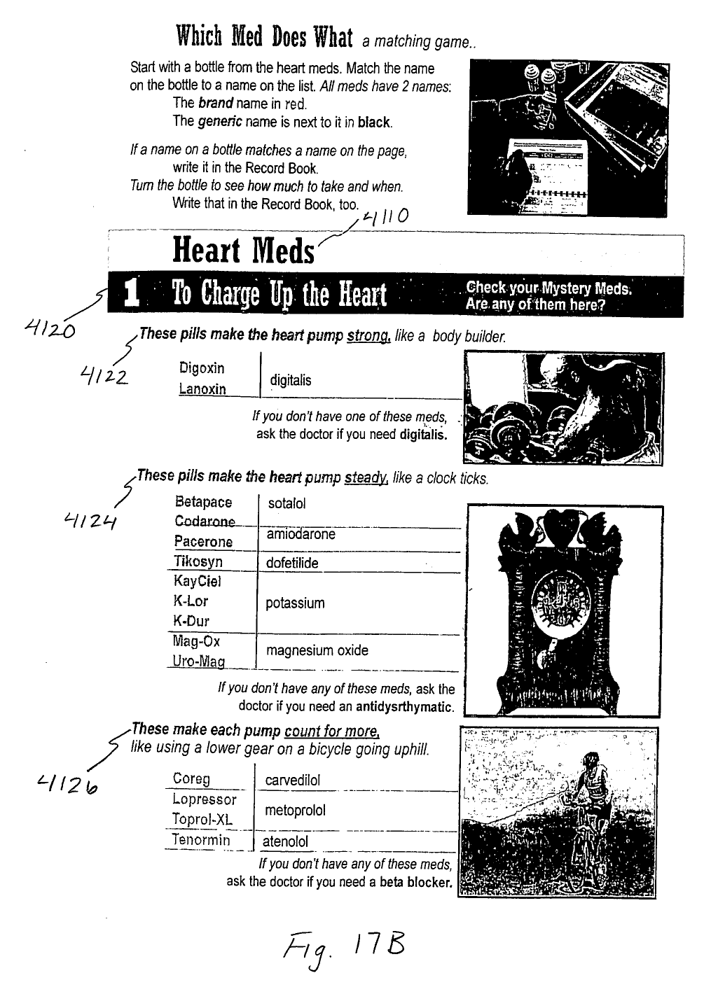

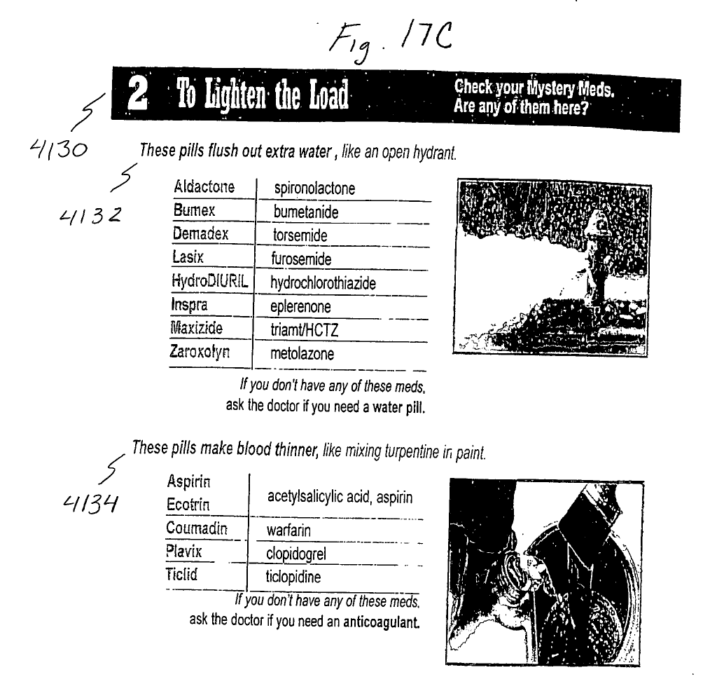

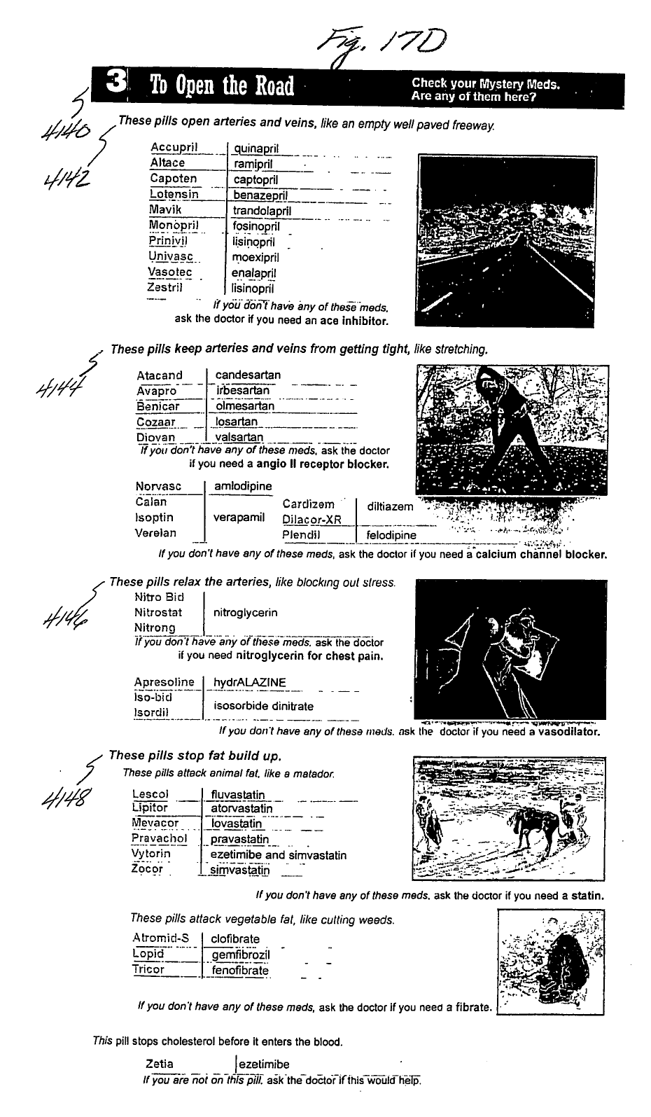

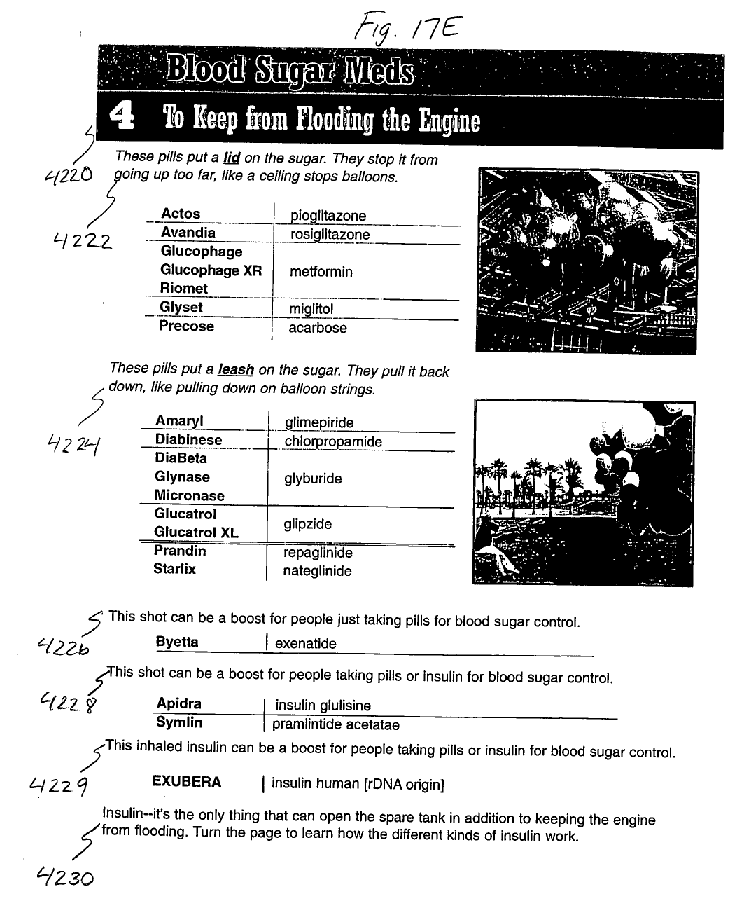

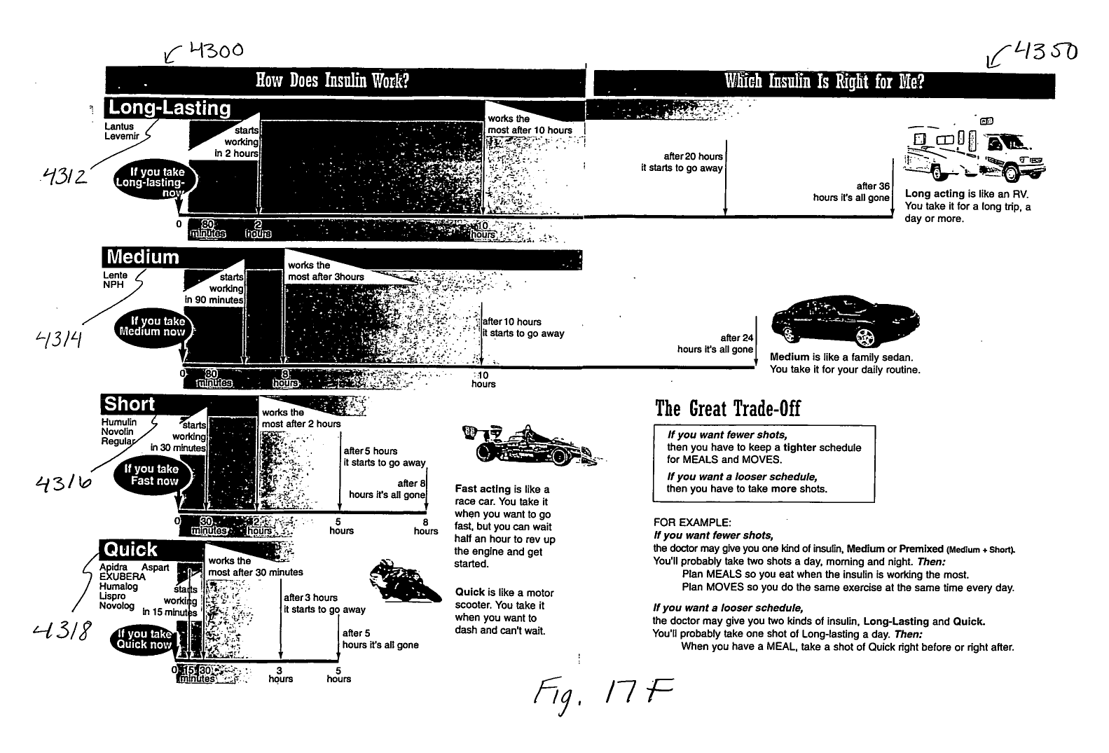

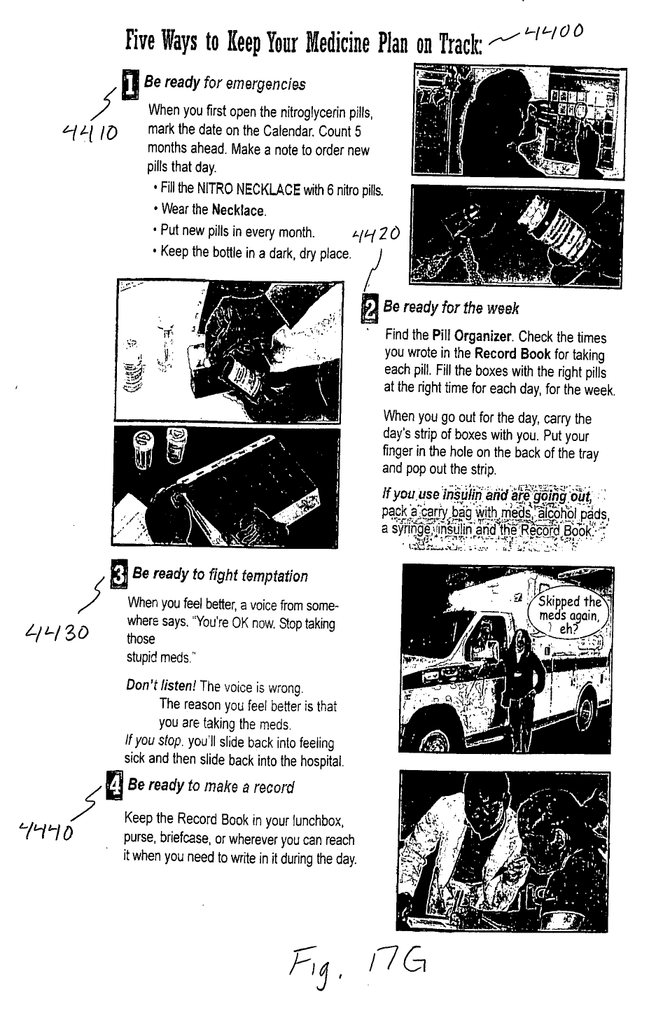

FIGS. 17A to 17G illustrate a secondary step-by-step medication guide 150 entitled, “The Right Meds at the Right Time.” The medication guide includes four sections. The first section 4010, illustrated in FIG. 17A, describes how the user can organize his or her medications. The second section 4100, illustrated in FIGS. 17A, 17B, 17C, 17D, 17E, and 17F provides an explanation to the user of what the medications are for. The third section 4400, illustrated in FIG. 17G, provides instructions to keep the medicine plan on track.

As illustrated in FIG. 17A, the medication guide instructs the user to organize the medications that the user is taking into five groups. The five groups include: medications that the doctor may not know the user has 4032 such as over-the-counter medications, vitamins, herbs and supplements; blood sugar medications 4034; heart medications 4036; other prescription medications 4038; and any “mystery” medications whose function is unknown to the user 4040. The medication guide 150 instructs the user to record each of the medications in the respective sections in the record book 106 as described above and as illustrated in FIG. 4E.

Once the user has organized the medications, the medication guide 150 instructs the user to understand what each medication is for 4100. The medication guide 150 categorizes each medication by whether it is a heart medication 4110 or blood sugar medication 4210 as illustrated in FIGS. 17B to 17F and provides a list of each of the drugs for each category.

The three types of heart medications include medications that charge up the heart 4120 illustrated in FIG. 17B, medications that lighten the load 4130 illustrated in FIG. 17C and medications that open the road 4140 illustrated in FIG. 17D. These three types of heart medications correspond to the concepts related to the heart presented in the education guide 112 described above and illustrated in FIGS. 3B and 3C. The medications that charge up the heart 4120 include pills that make the heart pump strong 4122 (i.e., digitalis), pills that make the heart pump steady 4124 (i.e., anti-disrhythmics) and pills that make each pump more efficient 4126 (i.e., beta blockers). Beta-blockers reduce a person's heart rate. The instructions liken this to using a lower gear on a bicycle to pedal uphill.

The system tries to get the user to understand that: (i) the heart medications that lighten the load 4130 include pills that flush out extra water 4132 (i.e., diuretics) and pills that make blood thinner 4134 (i.e., anti-coagulants), (2) diuretics or water pills flush out extra water from the body; and, (3) with less fluid to pump, the heart does not have to work as hard. The system and method disclosed herein provides this in an easy to understand and remember form to better enable the user to understand why they need to do certain things and show things are related.

The heart medications that open the road 4140 include pills that open arteries and veins 4142 (i.e., ace inhibitors), pills that keep arteries and veins from getting tight 4144 (i.e., angio II receptor blockers and calcium channel blockers), pills that relax the arteries 4146 (i.e., nitroglycerin and vasodilators), and pills that stop fat build-up 4148 (i.e., statins and fibrates). Ace inhibitors facilitate the opening of blood vessels. Calcium channel blockers relax a person's veins and arteries, which makes the veins and arteries wider and easier for blood to pass through. Nitroglycerin works quickly to relax a person's veins and arteries. Nitroglycerin is especially helpful to reduce chest pain by getting blood to the heart quickly. Statins reduce the amount of cholesterol the body produces. Fibrates reduce the amount of cholesterol that gets into the blood stream.

The medications that keep the user from “flooding the engine” 4220 are the blood sugar medications 4210. The list of blood sugar medications 4210 is illustrated in FIG. 17E and includes pills that prevent blood sugar from increasing too much 4222, pills that decrease blood sugar 4224, shots for people taking pills for blood sugar 4226, shots for people taking pills or insulin for blood sugar 4228, and insulin 4230.

The medication guide 150 includes a discussion 4300 on how insulin works 4310 and which insulin is right for the user 4350. This discussion is illustrated in FIG. 17F. The medication guide 150 graphically compares the onset and duration of the action and effects of each type of insulin. There are four different types of insulin. There is long-lasting 4318, medium 4314, short 4316, and quick 4318 types of insulin. To simplify the concepts for the user, the medication guide 150 also provides an analogy to different types of vehicles to illustrate the onset and duration of action of insulin. The medication guide 150 also discusses the relationship between the number of shots and adherence to the meal and exercise program 4320. For example, if a user wants to take fewer insulin shots, the guide recommends the user to plan to eat meals when the insulin is working the most, and plan exercise at the same time everyday. The user may only be given one kind of insulin, such as a medium or combination of a medium and short type of insulin. Alternatively, if the user wants a more flexible schedule, the doctor may give the user two kinds of insulin, long-lasting and quick insulin. The long-lasting insulin will be administered once a day and the quick insulin administered before or after a meal.

Referring to FIG. 17G and the “Four Ways to Keep Your Medicine Plan on Track” section 4400, the secondary step-by-step medication guide 150 includes instructions on being ready for the week 4410, being ready for “feeling traps” 4420, being ready to make a record 4430, and being ready for clinic visits 4440.

FIG. 17G also illustrates instructions on being ready for each week 4420. The instructions 4420 inform the user to pick one day a week (e.g., every Monday) to fill the pill organizer 154. The record book 106 may be used to ensure the correct pills are put into the pill organizer 154 in a way that corresponds to the right time of day for each pill to be taken. A portion of the pill organizer 154 corresponding to four time periods for each day (e.g., morning, noon, evening, bedtime) may be removed from the pill organizer 154 at the beginning of each day and replaced each night.

FIG. 17G also illustrates instructions on being ready for the “Feeling Traps” to fight temptation 4430 and to make a record 4440. The instructions 4430 inform the user that feeling better is not a reason to stop taking medications. On the contrary, the reason the person is feeling better is because he/she is taking the medications. A cycle that some people enter is to stop taking medications when they feel better only to wind up back in the hospital. The instructions 4440 also instruct the user to keep the record book 106 available to enter information.

FIG. 17G also illustrates instructions on being ready for doctor visits 4450. The instructions 4450 inform the user to record medication consumption in the record book 106 and to bring the record book 106 and the user's medication containers to each doctor visit. In this manner, the doctor can review the record book 106 and medication containers to determine if the right medications are being taken in the right quantities and frequencies.

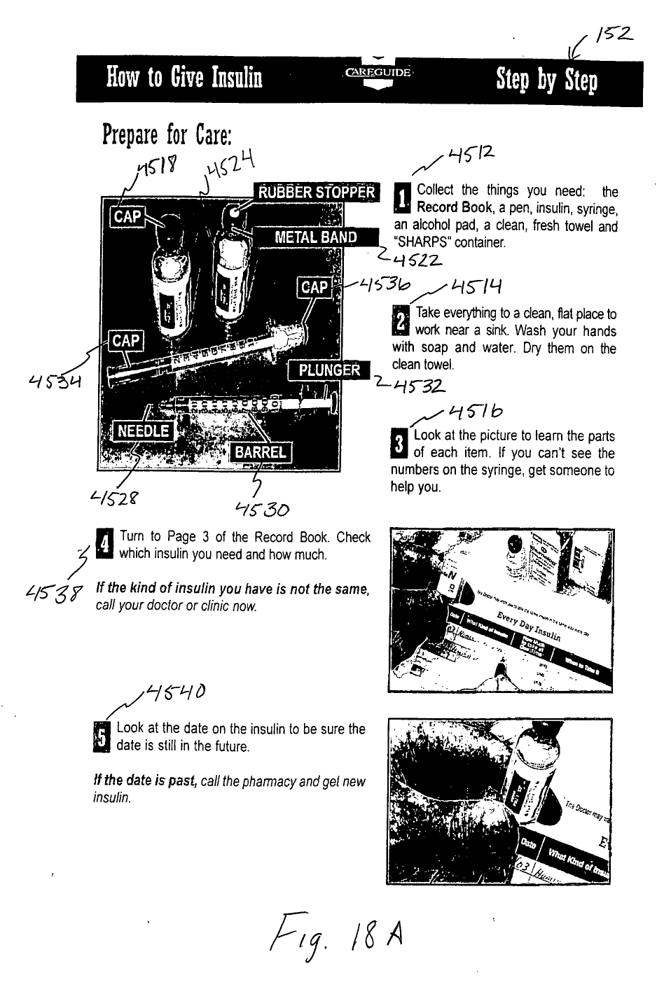

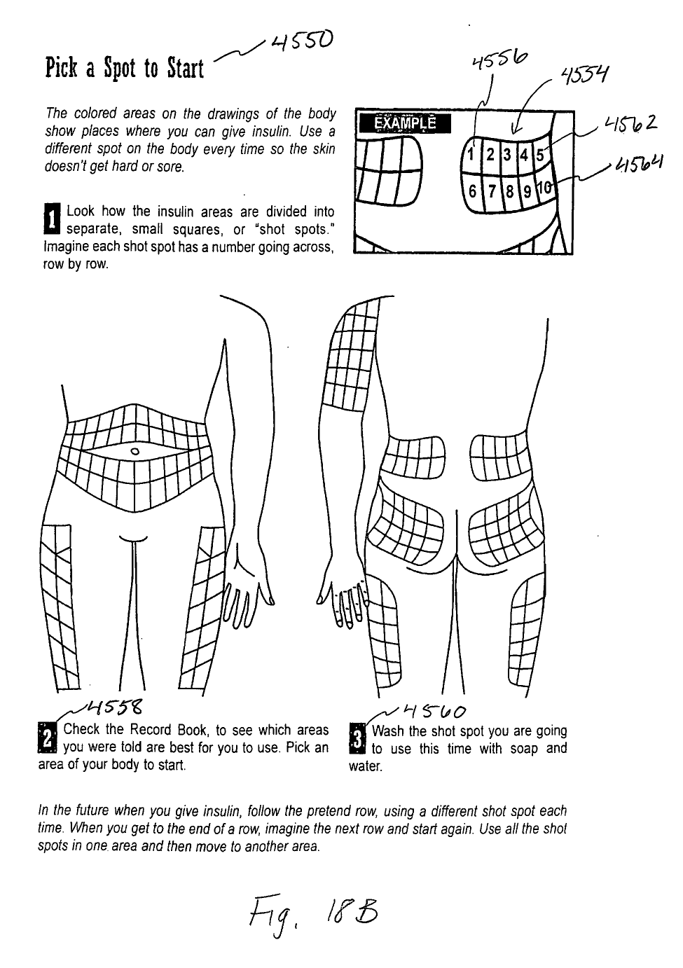

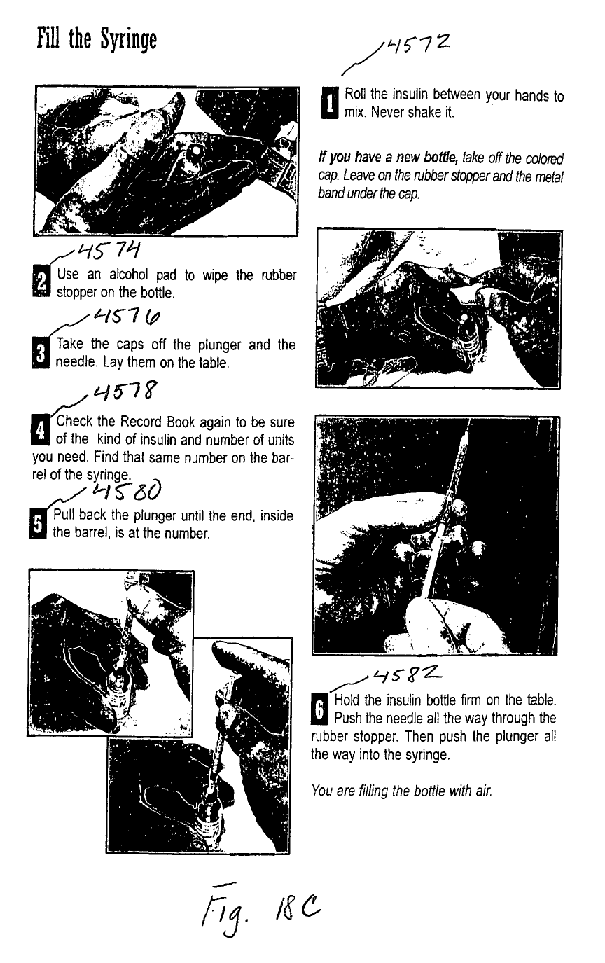

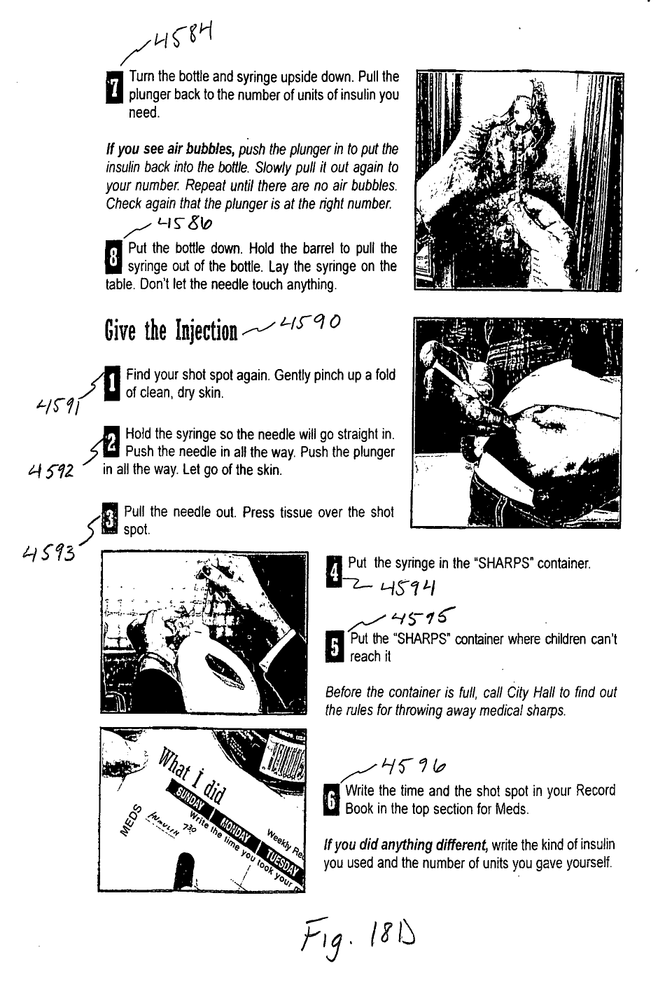

The system 100 includes an insulin administration guide 152 on how to give insulin illustrated in FIGS. 18A to 18D. The instruction guide includes four sections: the “Prepare for Care” section 4510, the “Pick A Spot To Start” section 4550, the “Fill The Syringe” section 4570 and the “Give the Injection” section 4580.

In the “Prepare for Care” section illustrated in FIG. 18A, five steps are listed in the instructions to prepare to administer insulin. The first step 4512 instructs the user to collect the things the user needs to administer insulin including the record book, a pen, insulin, syringe, an alcohol pad, a clean fresh towel and a “sharps” container. The second step 4514 includes instructing the user to take the items to a clean flat place near a sink to work and to wash hands with soap and water and dry them on a clean towel. The third step 4516 instructs the user to look at the picture to learn the parts of each item. To this end, the instruction guide includes illustrations of components used in administering insulin including a vial of insulin and a syringe, the cap 4518, rubber stopper 4520 and metal band 4522 of a vial 4524 of insulin are identified. Also included is an illustration of a syringe 4526 and the needle 4528, barrel 4530, and plunger 4532 of the syringe, along with the needle cap 4534 and the plunger cap 4536. The fourth step 4538 of the instructions instructs the user to reference the page in the record book 106 illustrated in FIG. 4E, as discussed above, where the user recorded the type of insulin and the amount of insulin to be administered. If the type of insulin the user has is not the same as that recorded in the record book 106, the insulin administration guide instructs the user to call the doctor or clinic immediately. The fifth step 4540 of the instructions advises the user to check the expiration date of the insulin.

The next section of the insulin administration guide includes the “Pick A Spot To Start” section 4550 illustrated in FIG. 18B. The instructions provide three steps in picking a spot to start. The first step 4552 instructs the user to refer to drawings indicating areas of the body where the user can give insulin, noting that the insulin administration areas 4554 illustrated are divided into separate small squares or “shot spots” 4556. Each shot spot 4556 is given a number going across row by row.

The second step 4558 instructs the user to check the record book 106 to see which doctor recommends areas are best to inject their insulin. The user is then instructed to pick an area 4554 of the body to begin a series of injections. The third step 4560 is to wash with soap and water the selected shot spot 4556 to be used. The user is instructed that a different spot on the body should be used each time an injection is given following a pretend row 4562. When the user comes to the end of a row 4562, the user is instructed to start on the next row 4564 using all the shot spots 4556 in one area 4554 before moving to another area.

The next section of instructions of the insulin administration guide includes a “Fill The Syringe” section 4570 illustrated in FIG. 18C. The “Fill The Syringe” section 4570 includes eight steps. The first step 4522 instructs the user to roll the insulin between the user's hands to mix and not to shake the insulin. The user is then instructed to take off the colored cap 4518 exposing the rubber stopper 4520 and metal band 4522 under the cap 4518 on the vial 4524. In the second step 4574, the user is instructed to use an alcohol pad to wipe the rubber stopper 4524 on the vial 4524. In the third step 4576, the user is instructed to take the caps 4536, 4534 off the plunger and the needle and lay them on the table. In the fourth step 4578, the user is instructed to check the record book 106 again to be sure the kind of insulin and number of units to be given is correct. The user is further instructed to find the same number on the barrel 4530 of the syringe 4526. The fifth step 4580 instructs the user to pull back the plunger 4532 until the end inside the barrel 4530 is at the number. The sixth step 4582 instructs the user to hold the insulin vial 4524 firm on the table, push the needle 4528 all the way through the rubber stopper 4520 and then push the plunger 4532 all the way into the syringe 4526 to fill the vial 4524 with air. The seventh step 4584 instructs the user to turn the vial 4524 and syringe 4526 upside down, and pull the plunger 4532 back to the number of units of insulin needed. If the user notices air bubbles, the user is instructed to push the plunger 4532 in to put the insulin back into the vial 4524. The instructions further instruct the user to slowly pull the plunger 4532 out again to the number of units needed and repeat until there are no air bubbles. The eighth step 4586 instructs the user to put the vial 4524 down and to hold the barrel 4530 to pull the needle 4528 out of the vial 4524. The instructions further provide that the user lay the syringe 4526 on the table preventing the needle 4528 from touching anything.

The fourth section of the insulin administration guide 152 includes the “Give The Injection” section 4590 includes six steps illustrated in FIG. 18D. The first step 4591 instructs the user to find the shot spot identified in the second section and to gently pinch up a fold of clean, dry skin. The second step 4592 instructs the user to hold the syringe so the needle will go straight in, push the needle in all the way, push the plunger in all the way injecting the insulin, and let go of the skin. The third step 4593 instructs the user to pull the needle out and press the tissue over the shot spot. The fourth step 4594 instructs the user to put the syringe in the sharps container. The fifth step 4595 instructs the user to place the sharps container where children cannot reach it. Before the container is full, the user is instructed to contact the local government to find out the rules for throwing away medical sharps. The sixth step 4596 instructs the user to document the time and shot spot 4556 in the record book 106 in the section for meds illustrated in FIG. 4E. If anything is done different than what is recorded in the record book 106, the user is instructed to record the kind of insulin used and the number of units given in the appropriate section of the record book 106 illustrated in FIG. 4I.

In the illustrated embodiment, the system 100 includes a pill organizer 154. The pill organizer 154 includes compartments for twenty-eight doses or four compartments for each of seven days of a week. The pill organizer 154 further includes labels indicative of each of the seven days and each of the four time periods of each of the seven days. It should be appreciated that any suitable pill organizer may be included in the system 100.

In addition, the illustrated embodiment of the system 100 includes glucose tablets 156. The glucose tablets may be in any suitable form and include any suitable dose.

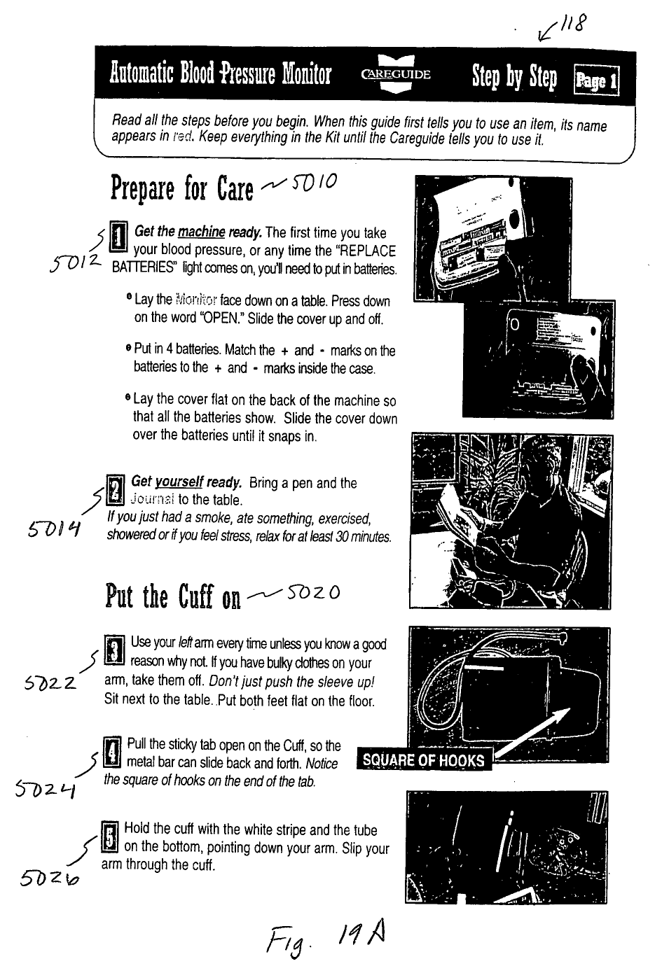

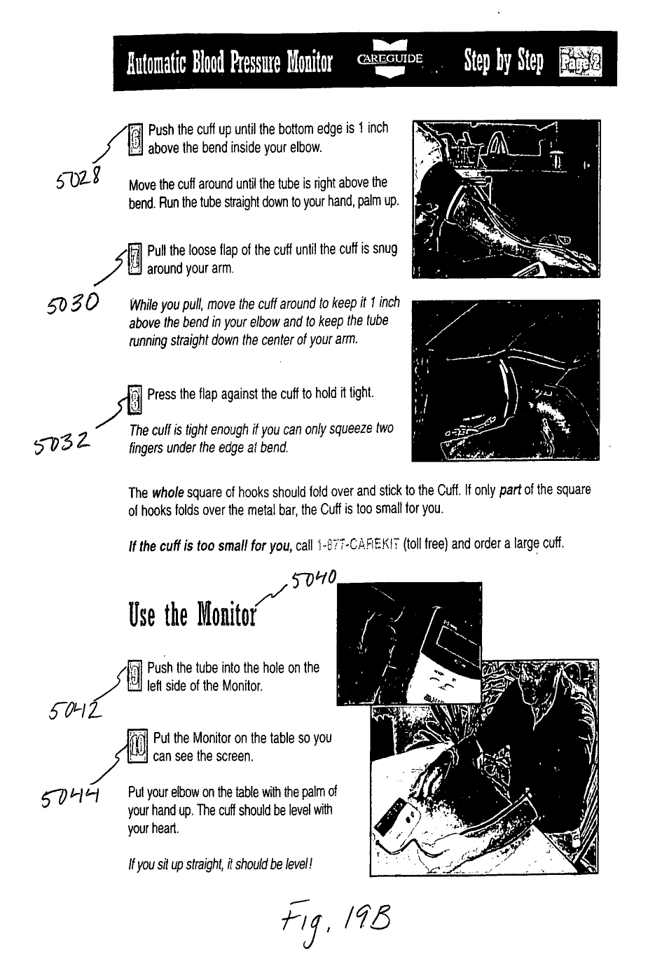

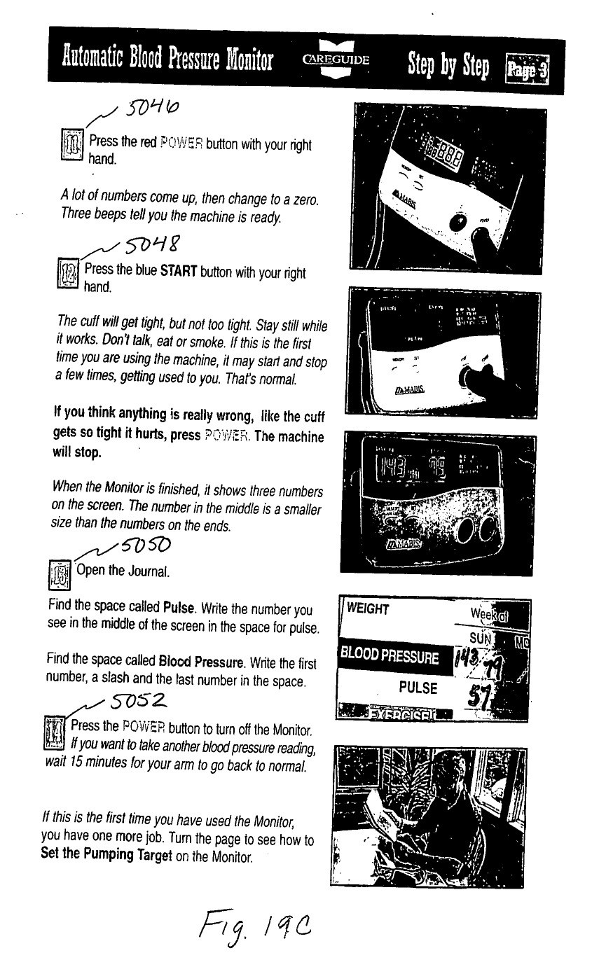

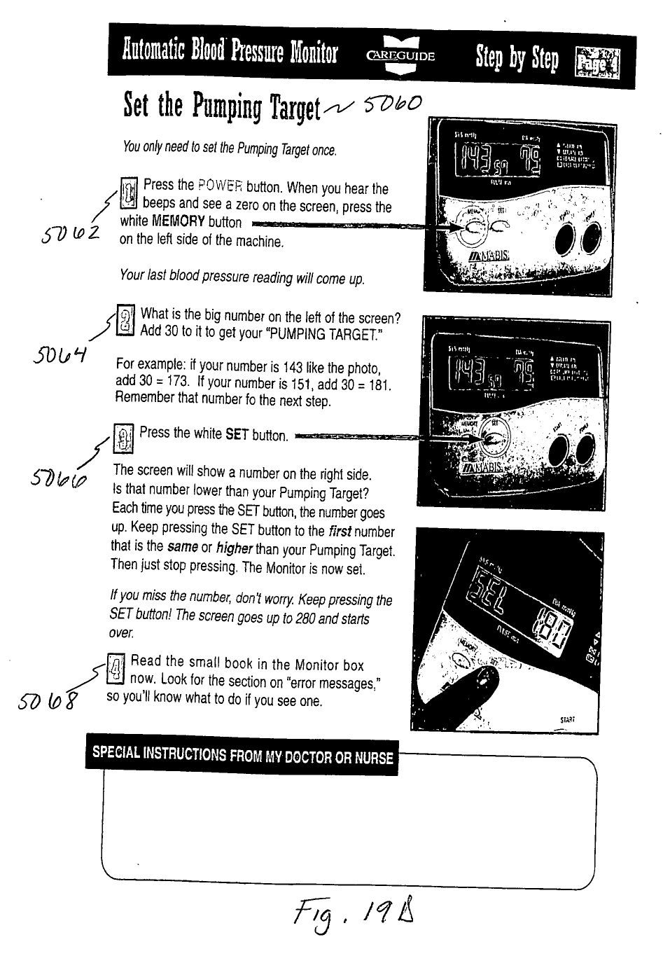

In the illustrated embodiment, the system 100 includes a blood pressure monitor 116 and instructions for the blood pressure monitor 118. Instructions 118 for the blood pressure monitor 116 are illustrated in FIGS. 19A to 19D.

The blood pressure monitor 116 may be any type of suitable blood pressure monitor. For example, the blood pressure monitor 116 may be a manual blood pressure monitor 116 or an automatic blood pressure monitor. A manual blood pressure monitor must be manually pumped (e.g., by hand). The instructions 118 are for an automatic blood pressure monitor, which pumps automatically. The instructions 118 indicate that the first time the user is instructed to use a particular item, the name of that item appears in red.

A first section 5010 of the blood pressure monitor instructions 118 tells the user how to prepare for a blood pressure reading. A first step 5012 tells the user to prepare the blood pressure monitor 116 for use by inserting fresh batteries. A second step 5014 tells the user to have a writing instrument and the medical record book 106 handy. In addition, the second step 5014 tells the user to relax for thirty minutes if he/she just smoked a cigarette, ate something, exercised, showered, or feels stressed.

A second section 5020 of the blood pressure monitor instructions 118 tells the user how to put the cuff on his/her arm. A third step 5022 tells the user to use the left arm (unless there is a good reason not to), remove bulky clothing, and to sit next to a table with both feet flat on the floor. A fourth step 5024 tells the user to pull open the sticky tab (e.g., velcro tab) on the cuff, so a metal bar can slide back and forth. A fifth step 5026 tells the user to slip his/her arm through the cuff while holding the cuff with the white strip and the tube on the bottom, pointing down the user's arm.

Turning to FIG. 19B, a sixth step 5028 tells the user to push the cuff up until the bottom edge of the cuff is about one inch above the bend inside the user's elbow. A seventh step 5030 tells the user to pull the loose flap against the cuff until the cuff is snug around the user's arm. An eighth step 5032 tells the user to press the flap against the cuff to hold the cuff tight.

A third section 5040 of the blood pressure monitor instructions 118 tells the user how to use the blood pressure monitor 116. A ninth step 5042 tells the user to push the tube into the hole on the left side of the monitor 116. A tenth step 5044 tells the user to place the blood pressure monitor 116 on the table where the display can be seen and to put the user's elbow on the table with the palm up and the cuff at the level of the user's heart.

Turning to FIG. 19C, an eleventh step 5046 tells the user to press the red power button on the blood pressure monitor 116 with the user's right hand. A twelfth step 5048 tells the user to press the blue start button on the blood pressure monitor 116 with the user's right hand. The twelfth step 5048 also explains that the cuff will tighten and explains how to read the numbers from the blood pressure monitor 116. A thirteenth step 5050 tells the user how to record the pulse and blood pressure readings in the medical record book 106. A fourteenth step 5052 tells the user to press the red power button again to turn the blood pressure monitor 106 off.

Turning to FIG. 19D, a fourth section 5060 of the blood pressure monitor instructions 118 tells the user how to set a pumping target on the blood pressure monitor 116. Typically, setting the pumping target only needs to be performed after the first use of the blood pressure monitor 116. A first step 5062 tells the user to turn the blood pressure monitor 116 on and press the white memory button. Pressing the memory button brings up the user's last blood pressure reading. A second step 5064 tells the user to calculate the user's actual pumping target by adding thirty to the last blood pressure reading. For example, if the last blood pressure reading was one hundred forty-three, adding thirty results in an actual pumping target of 173. A third step 5066 tells the user to press the set button and check the set target number that appears on the right hand side of the screen. If the set target number is lower than the actual pumping target, then the user is instructed to keep pressing the set button until the set target number reaches or exceeds (for the first time) the actual pumping target. A fourth step 5068 tells the user to consult a list of error messages to further familiarize the user with the blood pressure monitor 116. The blood pressure monitor instructions 118 also include a blank portion 5070 where the user may record any special instructions from his/her doctor about the blood pressure monitor 116 and/or taking the blood pressure readings.

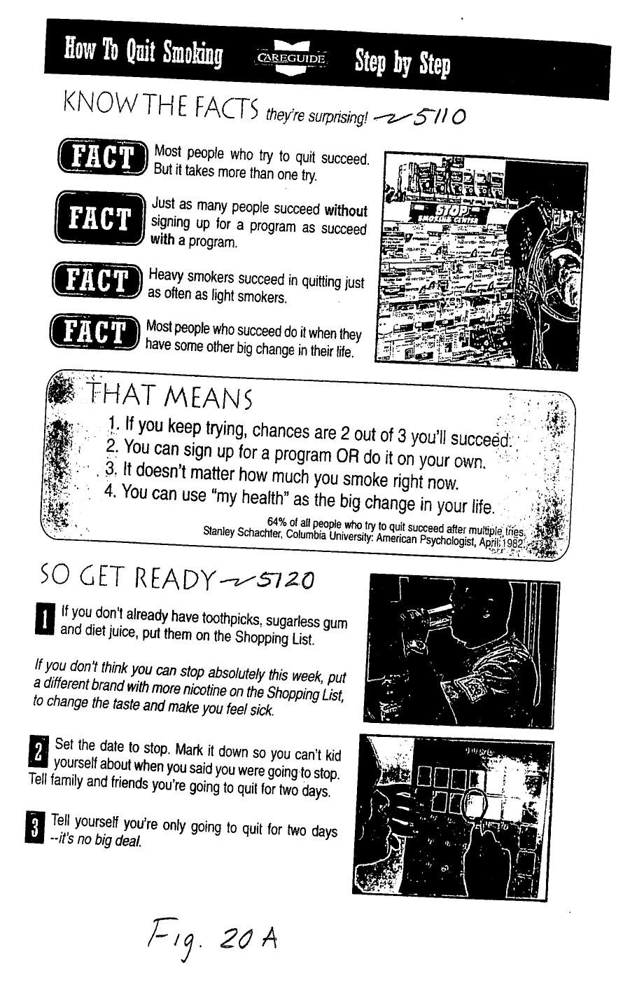

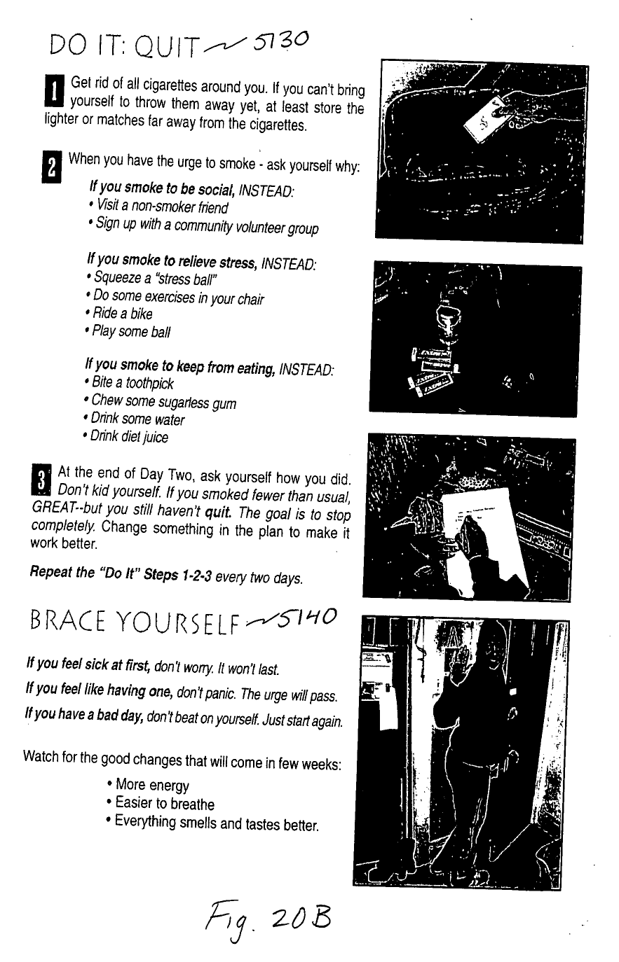

FIGS. 20A and 20B illustrate a secondary step-by-step tobacco addiction guide 158 included in the system 100 illustrated embodiment of the guide to assist the user in stop smoking. The tobacco addiction guide 158 includes facts about quitting smoking 5110, instructions on preparing to quit smoking 5120, instructions on quitting smoking 5130, and instructions on preparing for the effects of quitting smoking 5140.

FIG. 20A illustrates facts about quitting smoking 5110. The facts 5110 inform the user that most people who attempt to quit smoking succeed. However, quitting may take more than one attempt. In fact, about two thirds of people who try to quit succeed after multiple attempts. The facts 5110 also inform the user that just as many people succeed in quitting smoking without signing up for a program as those who succeed with a program. The facts 5110 also inform the user that heavy smokers succeed in quitting just as often as light smokers. So, it does not matter how much the person currently smokes. The facts 5110 also inform the user that most people who successfully quit smoking quit when they have some other big change in their life. For example, beginning to use the system 100 may be the big change.

FIG. 20A also illustrates instructions on preparing to quit smoking 1504. The instructions 5120 instruct the user to add toothpicks, sugarless gum, and diet juice to the person's grocery shopping list. In addition, the instructions 5120 suggest that if the person does not feel that he/she can stop smoking right away, to switch to a brand of cigarettes that contain more nicotine to change the taste associated with smoking and make the person feel sick. The instructions 5120 also tell the user to pick a date to stop smoking completely and to mark that date on their calendar. The instructions 5120 also instruct the user to tell friends and family (and himself/herself) that he/she only intends to quit for two days (i.e., that quitting is not a big deal).

FIG. 20B illustrates instructions on quitting smoking 5130. The instructions 5130 instruct the user to dispose of all cigarettes. Alternatively, the user may store any existing cigarettes in a separate place, away from lighters and matches. When the urge to smoke occurs, the instructions 5130 have the user ask himself/herself why they smoke. If the user smokes to be social, the instructions 5130 suggest visiting a non-smoking friend and/or joining a community volunteer group. If the user smokes to relieve stress, the instructions 5130 suggest squeezing a stress ball, doing some exercises (e.g., in the user's chair at work and/or at home), riding a bike, and/or playing a sport. If the user smokes to keep from eating, the instructions 5130 suggest biting on a toothpick, chewing sugarless gum, drinking some water, and/or drinking diet juice. After two days, the user performs a self-assessment and makes any adjustments that are needed to quit smoking.

FIG. 20B also illustrates instructions on preparing for the effects of quitting smoking 5140. The instructions 5140 inform the user that if he/she feels sick, not to worry because the feeling will pass. The instructions 5130 also inform the user that if the user feels the urge to have a cigarette, not to panic because the urge will pass. The instructions 5140 also inform the user that if the user has a bad day, not to worry and to just start the program over again. The instructions 5140 encourage the user by informing the user that by quitting smoking, the user should notice several positive benefits such as having more energy, having an easier time breathing, and that things will smell and taste better.

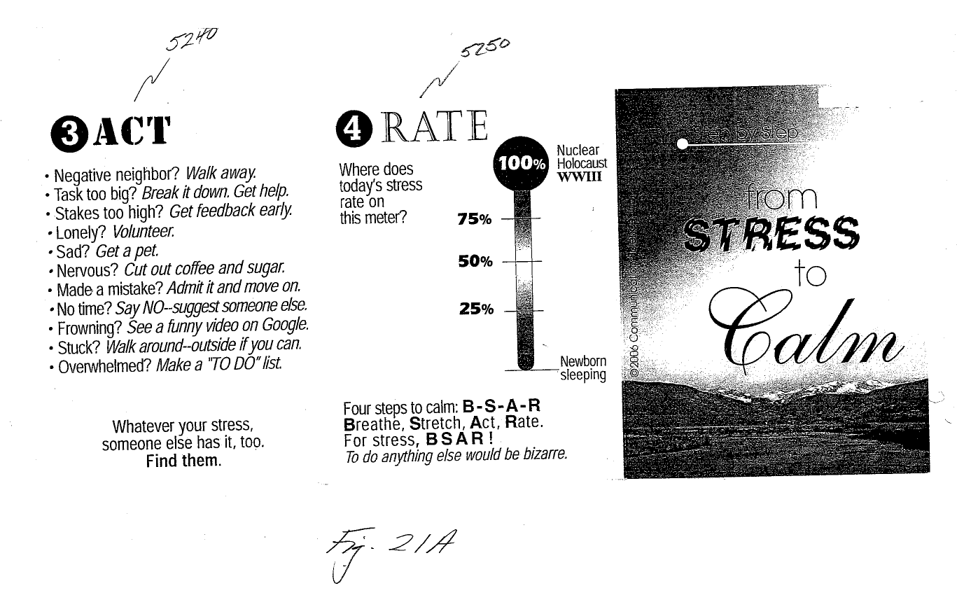

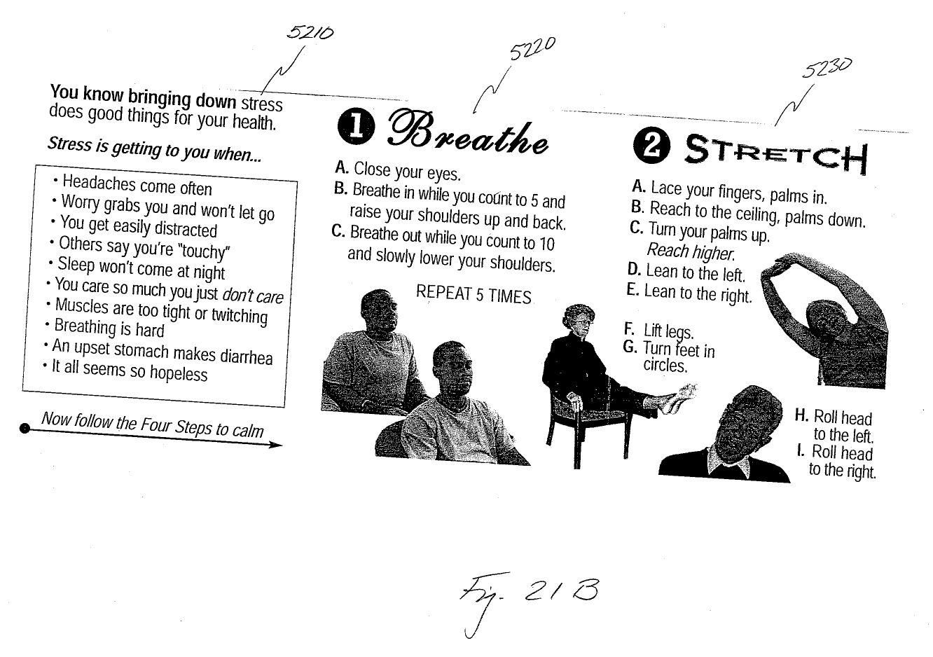

FIG. 21 illustrates a secondary step-by-step guide 160. The stress management guide 160 includes an explanation of how to recognize stress 5210 and ways to manage stress such as breathing exercises 5220, stretching exercises 5230, and actions to avoid stress 5240, and how to rate stress 5250.

The explanation of how to recognize stress 5210 informs the user that everyone experiences some stress. Stress is the body's natural reaction to tension, pressure, and/or change. Small amounts of stress make life more interesting and less boring. However, excess stress, especially prolonged and unrelieved stress, can be mentally and physically unhealthy. The stress management guide includes a listing of signs of stress 5210 such as headaches, upset stomach, hopeless feelings, etc. Many of these symptoms may also be caused by medical conditions, but the guide 160 points out that effective management of the stress may improve health. In addition, the stress management guide 160 may include a scale on which to rate the stress of the user at a particular time 5250.

The guide 160 includes anti-stress exercises such as breathing exercises 5220 and stretching exercises 5230. For example, the breathing exercises may include closing eyes, and breathing in or out for periods of time while moving certain parts of the body such as the shoulders. The stretching exercises may include a finger fan exercise, an upper back stretch, an ear-to-shoulder exercise, an overhead reach exercise, a knee-pull exercise, and a waist-bend exercise. The finger fan exercise includes extending the arms and spreading the fingers. The upper back stretch includes sitting up straight with the fingers interlocked behind the head and bring the elbows back. The ear-to-shoulder exercise includes lowering the ear to the shoulder. The overhead reach exercise includes raising the arms over the head with interlaced fingers. The knee-pull exercise includes pulling one knee at a time up to the chest in a seated position. The waist-bend exercise includes bending from side-to-side at the waist with the arms extended over the head.

The instructions on ways to manage stress 5240 include actions such as thinking positively and being around other positive people, avoiding being overly demanding on oneself and getting help when needed, writing and following a reasonable to do list each day, dividing big tasks into smaller more manageable tasks, eating a healthier diet, minimizing and/or eliminating consumption of coffee and sugar, getting plenty of sleep, getting enough exercise, making time to relax, avoiding tobacco, alcohol, and drugs, using mistakes and setbacks as opportunities to learn, avoiding high stress tasks, talking to a friend about disappointments and frustration before they build up and sharing your successes, admitting when you are wrong, eliminating everyday sources of stress such as loud music and clutter, having fun and laugh, knowing it is okay to cry, practicing deep breathing, being active during everyday tasks such as taking the stairs instead of the elevator and/or not sitting when talking on the telephone, and doing anti-stress stretches every day.

The stress management guide 160 may include other examples of how to act in response to stressful circumstances 5240 such as those listed in FIG. 21: if you have a negative neighbor, walk away, if a task is too big, break it down and get help; if the stakes are too high, get feedback early; if lonely, volunteer; if sad, get a pet; if nervous, cut out coffee and sugar; if a mistake is made, admit it and move on; if no time, say no and suggest someone else; if frowning, enjoy some comedy; if feeling stuck, walk around, outside if possible; if overwhelmed, make a “to do” list. These an other such examples may be included in the stress management guide 160.

It should be appreciated that any of the materials included in the system 100 may be included in any suitable form or format. Elements of the system may be included as a software product, stored on any suitable paper form, in a computer readable form storage device such as a CD-ROM, DVD. The instructions may be provided in additional video form.

It should be appreciated that the disclosed integrated system or any elements thereof may be provided in any suitable sensory form and on any suitable medium and combinations thereof. For example, any element of the disclosed integrated system may be provided in audio, visual, or tactile form such as Braille. Any element of the disclosed integrated system may be provided on paper, on any suitable machine or computer readable form such as CD-ROM, DVD, or any other suitable physical or electronic medium.

It should be appreciated that the system, apparatus and method disclosed herein provides: (a) an easy to understand and remember method for better enabling the user to understand why they need to do certain things and how things are related, (b) instructions which integrate the care for multiple medical conditions, and (c) directions on meal planning and other activities for multiple medical conditions, all without overlapping for contrary instructions. The system and apparatus are thus configured to enable a user to simultaneously provide integrated self care for multiple different medical conditions including diabetes, high blood pressure and heart failure.

In summary, methods and apparatus for blood sugar control, blood pressure control and heart failure care have been provided. The foregoing description has been presented for the purposes of illustration and description. It is not intended to be exhaustive or to limit the invention to the exemplary embodiments disclosed. Many modifications and variations are possible in light of the above teachings. It is intended that the scope of the invention be limited not by this detailed description, but rather by the claims appended hereto.

The invention is claimed as follows:

1. An integrated blood sugar control, blood pressure control and heart failure self-care system, the system comprising:
a magnet board, the magnet board including a portion to write a telephone number and a label indicating the telephone number is a doctor's telephone number;
a blood pressure monitor;
a pill organizer;
a stress management guide;
a primary step-by-step guide, the primary step-by-step guide including a plurality of primary steps associated with heart, blood pressure and blood sugar including at least three of:
(1) a dietary program;
(2) a medicine compliance program;
(3) a tobacco addiction program;
(4) a stress management program;
(5) an exercise program;
(6) a skin and foot care program; and
(6) a medical record keeping program; and
at least one secondary step-by-step guide, the secondary step-by-step guide including a plurality of secondary steps associated with at least one of the primary steps; and
a container for storing the above components.

2. The system of claim 1, wherein the blood pressure monitor includes a digital blood pressure monitor.

3. The system of claim 1, including a set of blood pressure monitor instructions, the blood pressure monitor instructions including at least three of:
(a) at least one instruction for getting the blood pressure monitor ready for use;
(b) at least one instruction for getting a user prepared;
(c) at least one instruction for attaching a cuff of the blood pressure monitor to the user; and
(d) at least one instruction for operating the blood pressure monitor.

4. The system of claim 1, wherein the pill organizer includes a twenty-eight dose pill organizer.

5. The system of claim 1, wherein the twenty-eight dose pill organizer includes: four compartments for each of seven days of a week, and labels indicative of each of the seven days and four time periods during each of the seven days.

6. The system of claim 1, wherein the stress management guide includes a pocket-sized stress management guide.

7. The system of claim 1, wherein the primary steps of the primary step-by-step guide includes steps associated with:
(1) the dietary program;
(2) the medicine compliance program;
(3) the tobacco addiction program;
(4) the stress management program;
(5) the exercise program; and
(6) the medical record keeping program.

8. The system of claim 1, wherein the plurality of secondary steps includes dietary program steps, the dietary program steps including at least three of:
(a) instructions on how to eat less salt;
(b) instructions on how to eat less cholesterol;
(c) instructions on how to eat carbohydrates;
(d) a food poster;
(e) a loose food guide;
(f) a fast food guide;
(g) a portion plate;
(h) a food selection game;
(i) a meal planner; and
(e) a shopping list.

9. The system of claim 1, wherein the plurality of secondary steps includes medicine compliance program steps, the medicine compliance program steps including at least two of:
(a) instructions on how to organize pills;
(b) information on the purpose of different types of pills; and
(c) instructions on how to administer insulin.

10. The system of claim 1, wherein the plurality of secondary steps includes tobacco addiction program steps, the tobacco addiction program steps including at least three of:
(a) facts about people who try to quit smoking;
(b) a shopping list;
(c) instructions on marking a calendar; and
(d) instructions on alternatives to smoking.

11. The system of claim 1, wherein the plurality of secondary steps includes stress management program steps, the stress management program steps including at least three of:
(a) an explanation of stress;
(b) a list of stress signs;
(c) a list of ways to manage stress; and
(d) a plurality of anti-stress exercises.

12. The system of claim 1, wherein the plurality of secondary steps includes exercise program steps, the exercise program steps including at least two of:
(a) information about the benefits of walking;
(b) a list of things needed for walking.

13. The system of claim 1, wherein the plurality of secondary steps includes medical record keeping program steps, the medical record keeping program steps including at least three of:
(a) medication compliance recording;
(b) weight recording;
(c) blood pressure recording;
(d) blood sugar recording;
(e) exercise recording;
(f) tobacco consumption recording; and
(g) pain recording.

14. The system of claim 1, wherein the plurality of secondary steps includes a medical record keeping step and the system includes a medical record keeping form, the medical record keeping form including at least three of:
(a) a place to record answers to questions posed to a doctor;
(b) a place to record blood test results;
(c) a place to record prescription medication information;
(d) a place to record blood pressure readings;
(e) a place to record blood sugar readings;
(e) a place to record body weights;
(f) a place to record tobacco consumption; and
(g) a place to record pain information.

15. A method for integrated blood sugar control, blood pressure control and heart failure care, the method comprising:
providing a blood pressure monitor;
providing a pill organizer;
providing a stress management guide;
providing a primary step-by-step guide, the primary step-by-step guide including a plurality of primary steps associated with heart, blood pressure and blood sugar including at least three of:
(1) a dietary program;
(2) a medicine compliance program;
(3) a tobacco addiction program;
(4) a stress management program;
(5) a skin and foot care program;
(6) an exercise program; and
(7) a medical record keeping program; and
providing at least one secondary step-by-step guide, the secondary step-by-step guide including a plurality of secondary steps associated with at least one of the primary steps.

16. A system for integrated blood sugar control, blood pressure control and heart failure self-care by a user, the system comprising:
a blood pressure monitor;
a primary step-by-step guide associated with the blood pressure monitor, the primary step-by-step guide including a plurality of primary steps associated with blood pressure, blood sugar and heart failure including at least five of:
(1) a dietary program;
(2) a medicine compliance program;
(3) a tobacco addiction program;
(4) a stress management program;
(5) a skin and foot care program
(5) an exercise program; and
(6) a medical record keeping program;
at least one secondary step-by-step guide, the secondary step-by-step guide including a plurality of secondary steps associated with at least one of the primary steps; and a container for storing the above components.
